(19) United States
(12) Patent Application Publication
(10) Pub. No.: US 2010/0178700 A1
(43) Pub. Date: Jul. 15, 2010

(54) NOVEL THYMIC CELLULAR POPULATIONS AND USES THEREOF

(75) Inventors: Anne Fletcher, Malvern (AU); Ann Chidgey, Black Rock (AU); Natalie Seach, Ringwood (AU); Richard Boyd, Hampton (AU)

Correspondence Address: KNOBBE MARTENS OLSON & BEAR LLP, 2040 MAIN STREET, FOURTEENTH FLOOR, IRVINE, CA, 92614, US

(73) Assignees: AUSTRALIAN STEM CELL CENTRE LTD., Clayton (AU); NORWOOD IMMUNOLOGY LIMITED, Malvern (AU)

(21) Appl. No.: 12/598,680

(22) PCT Filed: May 2, 2008
(86) PCT No.: PCT/AU08/00615

§ 371 Date: Mar. 12, 2010

(30) Foreign Application Priority Data
May 3, 2007 (AU) 2007902337

(51) Int. Cl. C12N 5/07

(52) U.S. Cl. 435/377; 435/325

(57) ABSTRACT

The present invention relates generally to novel thymic cellular populations and, more particularly, to novel thymic epithelial cellular populations. Most particular, the present invention is directed to novel thymic epithelial progenitor cell populations. The cellular populations of the present invention are useful in a wide range of clinical and research settings including, inter alia, the in vitro or in vivo generation of thymic epithelial cell populations and the therapeutic or prophylactic treatment of a range of conditions via the administration of these cells. Also facilitated is the design of in vitro based screening systems for testing the therapeutic impact and/or toxicity of potential treatment or culture regimes to which thymic epithelial cells may be exposed. In another aspect, the present invention is directed to a method of identifying thymic epithelial cellular subpopulations and, more particularly, thymic epithelial progenitors by screening for the co-expression of markers including MHC Class II, UEA1 and Ly51. This method is useful in a range of applications including, but not limited to, assessing or monitoring for the presence of thymic epithelial cell populations and/or facilitating the isolation of or enrichment for these cellular populations of use in a range of research and clinical applications.

### FIELD OF THE INVENTION

The present invention relates generally to novel thymic cellular populations and, more particularly, to novel thymic epithelial cellular populations. Most particular, the present invention is directed to novel thymic epithelial progenitor cell populations. The cellular populations of the present invention are useful in a wide range of clinical and research settings including, inter alia, the in vitro or in vivo generation of thymic epithelial cell populations and the therapeutic or prophylactic treatment of a range of conditions via the administration of these cells. Also facilitated is the design of in vitro based screening systems for testing the therapeutic impact and/or toxicity of potential treatment or culture regimes to which thymic epithelial cells may be exposed. In another aspect, the present invention is directed to a method of identifying thymic epithelial cellular subpopulations and, more particularly, thymic epithelial progenitors by screening for the co-expression of markers including MHC Class II, UEA1 and Ly51. This method is useful in a range of applications including, but not limited to, assessing or monitoring for the presence of thymic epithelial cell populations and/or facilitating the isolation of or enrichment for these cellular populations of use in a range of research and clinical applications.

### BACKGROUND OF THE INVENTION

Bibliographic details of the publications referred to by author in this specification are collected alphabetically at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The development of T cells that bear the αβ form of the T-cell receptor occurs exclusively within the thymus. Intrathymic T-cell maturation proceeds from fetal liver or bone marrow-derived haemopoietic stem cells and occurs via a differentiation program readily characterized by changes to cell-surface phenotype, proliferation status and functionality. Key events in T-cell development include lineage commitment, selection events, and thymic emigration.

The thymus arises from a common embryonic region that develops from the third pharyngeal pouch [Gordon et al. Mech Dev, 2001, 103:141-143]. The process of thymic organogenesis is generally divided into two stages with the early stage being characterised by epithelial-mesenchymal interactions in the absence of thymocytes and the latter stage involving mutual interactions between epithelial cells and developing thymocytes [Nehls et al., Science 1996, 272:886-889; Manley N R., Semin Immunol 2000, 12:421-428; Blackburn and Manley, Nat Rev Immunol 2004, 4:278-289; Boehm et al., J Exp Med 2003, 198:757-769; Pongracz et al., Eur J Immunol 2003, 33:1949-1956; Owen et al., Curr Top Microbiol Immunol 2000, 251:133-137]. The thymic rudiment is first visible at about embryonic day 10.5 (E10.5), and it has been suggested that all the three germ layers contribute to thymic organogenesis. The initiating signals for thymus organogenesis remain elusive. However, it has been well documented that mesenchymal cells play critical roles at different stages of thymus organogenesis [Suniara et al., J Exp Med 2000, 191:1051-1056; Owen et al. Curr Top Microbiol Immunol 2000, 251:133-137] and there exists a body of evidence suggesting that mesenchymal cells may respond to the initiating instructive signals for thymus induction from the endoderm and in turn support the growth and differentiation of the thymic epithelial rudiment. At E10, neural crest-derived mesenchymal cells initiate physical interaction with the third pharyngeal pouch and subsequently establish the thymic primordium. At about E11, the thymic rudiment begins budding and outgrowth, followed by immigration of the thymocyte precursors.

In terms of the process of thymocyte differentiation, the earliest precursors present within the murine thymic microenvironment are defined as CD4^{lo}c-kit⁺CD44⁺Thy-1⁻Sca-1⁺ [Wu et al., Nature 1991, 349:71-74]. However, these cells are not fully T-lineage restricted and can give rise to both B cells and myeloid cells (dendritic cells) [Matsuzaki et al., J Exp Med 1993, 178:1283-1292; Ardavin et al., Nature 1993, 362:761-763]. The CD4^{lo} precursor thymocytes are often described as triple negative cells (CD3⁻CD4⁻CD8⁻) and can be further subdivided based on their expression of CD44 [phagocytic glycoprotein-1 (pgp-1)], CD25 [interleukin-2 receptor α (IL-2Rα) chain], CD117 (c-kit), and CD127 (IL-7Rα chain). The most immature triple negative subset (TN1) requires many cytokines for survival and can be classified as CD44⁺CD25⁻CD117⁺CD127⁻. These cells acquire CD25 and CD127 and upregulate expression of the gene encoding pre-TCRα (pTα), to become CD44⁺CD25⁺CD117⁺CD127⁺ (TN2), in which IL-7 alone has been shown to be sufficient for survival. At this stage, TCR gene rearrangement is initiated and the ability to develop into B cells in vivo is ultimately lost [Rodewald et al., EMBO J 1994, 13:4229-4240; Godfrey et al., Immunol Today 1993, 14:547-553; Groettrup et al., Cell 1993, 75:283-294; Moore et al., Blood 1995, 86:1850-1860; Wu et al., Am J Reprod Immunol 1996, 35:510-516].

Thymocytes that have undergone productive TCRβ chain rearrangements are selected for expansion and further maturation prior to expression of the TCRα chain. This process is termed β-selection and is controlled by the pre-TCR complex. The pre-TCR complex consists of the rearranged TCRβ chain together with an invariant pTα chain and a number of non-covalently associated polypeptides referred to as the CD3 complex [Groettrup et al., Cell 1993, 75:283-294; Fehling et al., Nature 1995, 375:795-798; reviewed in Wiest et al., Semin Immunol 1999, 11:251-262]. The triple negative cells eventually become double positive CD4⁺CD8⁺CD3^{lo/int} via CD4 or CD8 immature single positives with a concomitant burst in proliferation [Hugo et al., Int Immunol 1990, 2:209-218; Tatsumi et al., PNAS 1990, 87:2750-2754; Godfrey et al., Immunology 1990, 70:66-74; Penit et al., J Immunol 1988, 140:3315-3323]. Following DP transition, developing thymocytes undergo extensive selection to ensure that the mature T cells that are exported from the thymus are functional (self-major histocompatibility complex (MHC) restricted) and self-tolerant. These processes are termed positive and negative selection and are dependent on lymphostromal interactions within the thymus (reviewed in Jameson and Bevan, Curr Opin Immunol 1998, 10:214-219; Anderson et al., Immunol Today 1999, 20:464-469; Sebzda et al., Annu Rev Immunol 1999, 17:829-874; Klein and Kyewski, Curr Opin Immunol 2000, 12:179-186; Chidgey et al., APMIS 2001, 109:481-492]. Positive selection is a consequence of whether or not the TCR on the developing thymocytes is able to recognize (self) peptide within the context of self-MHC (reviewed in von Boehmer H., Cell 1994, 76:219-228; Bevan, Immunity 1997, 7:175-178]. Those that cannot recognize this self MHC-peptide complex die by neglect. In contrast, thymocytes with too high an affinity for self-MHC peptide are deleted by negative selection, an active apoptotic process designed to remove self-reactive clones [Smith et al., Science 1989, 245:749-752, reviewed in Nossal, Cell 1994, 76:229-239; Sprent et al., Immunol Rev 2002, 185:126-135]. The remaining thymocytes that respond with low affinity are selected to develop into CD4⁺CD8⁻ or CD4⁻CD8⁺ single-positive thymocytes, with concomitant downregulation of the CD8⁺ or CD4⁺ coreceptor, respectively [Kaye et al., Nature 1989, 341:746-749; Merkenschlager et al., J Exp Med 1997, 186:1149-1158]. Development into CD4⁺ T-helper cells requires interaction with MHC Class II-expressing thymic stromal cells, while development into CD8⁺ cytotoxic T lymphocytes (CTLs) requires interaction with MHC Class I-expressing thymic stromal cells [Kaye et al. 1989 supra; Teh et al., Nature 1988, 335:229-233].

Those thymocytes surviving selection mature in the medulla for an average of 14 days before thymic export [Scollay and Godfrey, Immunol Today 1995; 16:268-273; discussion 273-264]. While the nature of this medullary maturation process is unknown, it is associated with phenotypic and functional changes in SP thymocytes. Recently selected thymocytes are HSA^{hi} CD69⁺CD24⁺CD62L⁻Qa-2⁻, while recent thymic emigrants (RTES) are HSA^{lo}CD69⁻CD24⁻CD62L⁺Qa-2⁺ [Gabor et al., Eur J Immunol 1997, 27:2010-2015]. Following this differentiation process, mature thymocytes are believed to migrate towards the post-capillary venules at the cortico-medullary junction of the thymus and are exported at a rate of 1-2% of total thymocytes per day [Scollay et al., Eur J Immunol 1980, 10:210-218; Berzins et al., Proc Natl Acad Sci USA 1999, 96:9787-9791].

Accordingly, once the thymus has developed, this organ is characterised by discrete subpopulations of epithelium, each exhibiting a distinct phenotype and arranged in a precise three dimensional configuration. Indeed, discrete epithelial microenvironments regulate each stage of thymocyte development. For example, the thymocyte selection steps of the thymocyte differentiation process are tightly regulated stages involving the presentation of MHC and self peptides by specific thymic epithelial subpopulations. The degree of affinity for self exhibited by each T cell receptor determines whether a thymocyte will mature and exit the thymus. Positive selection is mediated by cortical thymic epithelial cells and ensures that mature T cells are self-MHC restricted while negative selection is arguably a more complicated process, during which dendritic cells or medullary thymic epithelial cells induce apoptosis in thymocytes showing a high degree of self-avidity. Together, positive and negative selection strike a balance to create a broadly reactive T cell receptor repertoire, with a low—but not absent—potential for self-reactivity.

Hence, functionally the cortex is considered to generate the T cell repertoire and the medulla to induce self-tolerance. Both thymic compartments are thus required to establish a fully functional immune system that defends against foreign infections whilst maintaining self integrity. The anomalous situation is the need to reject cancers which are a disease of self and yet not react against normal self. The former obviously often fails yet autoimmune disease is common. A further complication is that the thymus undergoes severe age-related atrophy creating gradually increasing immunodeficiency, predisposing the individual to opportunistic infection and increase in cancer incidence and burden. Paradoxically there is also an increase in autoimmune disease with age. A further complication of cancer is the need for chemotherapy, which not only severely depletes the immune system, but also causes additional damage to the thymic infrastructure, exacerbating the T cell depletion and inability to restore these cells.

The thymus is therefore a complex epithelial organ in which thymocyte development is dependent upon the sequential contribution of morphologically and phenotypically distinct stromal cell compartments. It is these microenvironments that provide the unique combination of cellular interactions, cytokines, and chemokines to induce thymocyte precursors to undergo the differentiation program that leads to the generation of functional T cells.

Understanding how these different functions of T cells are dictated by the thymus is predicated by an understanding of how the thymus cortex and medulla arise. Accordingly, fundamental to reversing thymic damage is an understanding of the identification of progenitor epithelial cells for the cortex and medulla. Nevertheless, despite the indispensable role of thymic epithelium in the generation of T cells, the mediators of this process and the differentiation pathway undertaken by the primordial thymic epithelial cells are not well defined.

In work leading up to the present invention, a thymic epithelial cell expressing low levels of MHC Class II has been both identified and determined to correspond to a population of thymic epithelial progenitor cells, the major subpopulation of which is a cortex and medulla “common” thymic epithelial progenitor cell (cmTEPC) which is able to give rise to both cortical and medullary epithelial cells. cmTEPC are MHC Class II low, UEA-1 low and Ly51/6C3 low. There also exist 2 further sub-populations, these corresponding to a medulla thymic epithelial progenitor cell (mTEPC) defined as MHC Class II low, UEA1 Lo, Ly51⁻ and a cortex thymic epithelial progenitor cell (cTEPC) defined as MHC Class II low, Ly51 low UEA1⁻. Finally, there has been identified an epcam⁺/MHC Class II⁻ epithelial progenitor cell population which is believed to function as the precursor to the epithelial MHC Class II^{lo} populations described above.

These thymic epithelial progenitor cells have been identified, isolated and characterised. Their phenotypic characterisation has enabled their reliable and routine identification and isolation. This development is very valuable when one considers that thymic epithelial progenitor cells are capable of differentiating to the cortical and medullary thymic epithelial subpopulations which are central to thymocyte education. Accordingly, the isolation of these cells now provides a means of prophylactically or therapeutically repairing thymic damage, such as that caused by chemotherapy, radiation, age or the like. Also facilitated are means of modulating thymic functionality, rationally designing tolerance induction, such as to facilitate a reduction in likely rejection of allogeneic transplantation or tissue grafting. Finally, there is provided means of generating thymic epithelial progenitor cell lines and the use of these cell lines in a range of screening techniques.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

One aspect of the present invention provides an isolated mammalian thymic epithelial progenitor cell expressing an MHCII^{lo} phenotypic profile or mutant or variant thereof.

Another aspect of the present invention provides an isolated mammalian thymic epithelial progenitor cell expressing a phenotypic profile of CD45⁻, epcam⁺ and MHC Class II^{lo} or mutant or variant thereof.

Still another aspect of the present invention provides an isolated mammalian thymic epithelial progenitor cell expressing a phenotypic profile selected from:

### (i) MHC Class II^{lo};

### (ii) MHC Class II^{lo}, UEA1^{lo} and Ly51^{lo}; or

(iii) MHC Class II⁻
or mutant or variant thereof.

Yet another aspect of the present invention provides an isolated mammalian thymic epithelial progenitor cell expressing a phenotypic profile selected from:

(i) MHC Class II^{lo}, CD45⁻ and epcam⁺;

### (ii) MHC Class II^{lo}, CD45⁻, UEA1^{lo} and Ly51^{lo}; or

(iii) MHC Class II⁻, CD45⁻ and epcam⁺
or mutant or variant thereof.

In another aspect, there is provided an isolated thymic cortical progenitor cell expressing a phenotypic profile of MHC Class II^{lo}, UEA1^{-/lo} and Ly51⁺ or mutant or variant thereof.

In still another aspect, there is provided an isolated thymic medullary progenitor cell expressing a phenotypic profile of MHC Class II^{lo}, UEA1⁺ and Ly51⁻ or mutant or variant thereof.

Yet another aspect of the present invention is directed to a method for identifying a mammalian thymic epithelial progenitor cell, said method comprising screening for MHC Class II, CD45, epcam, UEA1 and/or Ly51 cellular expression in a mammal or in a biological sample derived from said mammal wherein expression of a phenotypic profile selected from:

(i) MHC Class II^{lo}, CD45⁻ and epcam⁺;

### (ii) MHC Class II^{lo}, CD45⁻, UEA1^{lo} and Ly51^{lo}; or

(iii) MHC Class II⁻, CD45⁻ and epcam⁺
is indicative of a thymic epithelial progenitor cell.

In another aspect, the present invention is directed to a method for identifying a mammalian thymic cortical progenitor cell, said method comprising screening for MHC Class II, CD45, UEA1 and Ly51 cellular expression in a mammal or in a biological sample derived from said mammal wherein expression of the phenotypic profile of MHC Class II^{lo}, CD45⁻, UEA1^{-/lo} and Ly51⁺ is indicative of a thymic cortical progenitor cell.

In still another aspect, the present invention is directed to a method for identifying a mammalian thymic medullary progenitor cell, said method comprising screening for MHC Class II, CD45, UEA1 and Ly51 cellular expression in a mammal or in a biological sample derived from said mammal wherein expression of the phenotypic profile of MHC Class II^{lo}, CD45⁻, UEA1⁺ and Ly51⁻ is indicative of a thymic medullary progenitor.

In a related aspect, there is provided a method for identifying a mammalian thymic epithelial progenitor cell, said method comprising screening for MHC Class II, UEA1, Ly51, epcam and/or CD45 expression by the cells of a biological sample wherein expression of a phenotypic profile selected from:
(i) MHC Class II^{lo}, CD45⁻ and epcam⁺;
(ii) MHC Class II^{lo}, CD45⁻, UEA1^{lo} and Ly51^{lo}; or
(iii) MHC Class II⁻, CD45⁻ and epcam⁺
   is indicative of a thymic epithelial progenitor cell.

In another aspect, there is provided a method for identifying a mammalian thymic cortical epithelial progenitor cell, said method comprising screening for MHC Class II, UEA1, Ly51, epcam and/or CD45 expression by the cells of a biological sample wherein expression of a MHC Class II^{lo}, CD45⁻, UEA1^{-/lo} and Ly51⁺ phenotypic profile by a cell is indicative of a thymic cortical epithelial progenitor cell.

In still another aspect, there is provided a method for identifying a mammalian thymic medullary epithelial progenitor cell, said method comprising screening for MHC Class II, UEA1, Ly51, epcam and/or CD45 expression by the cells of a biological sample wherein expression of a MHC Class II^{lo}, CD45⁻, UEA1⁺ and Ly51⁻ phenotypic profile by a cell is indicative of a thymic medullary epithelial progenitor cell.

Still another aspect of the present invention provides a kit for identifying a thymic epithelial progenitor cell of the present invention, said kit comprising an agent for detecting MHC Class II, UEA1, Ly51, epcam and/or CD45 cell surface expression or MHC Class II, UEA1, Ly51, epcam and/or CD45 mRNA expression and, optionally, reagents useful for facilitating the detection by said agent.

Yet another aspect of the present invention provides a method for isolating a mammalian thymic epithelial progenitor cell, said method comprising the steps of:
(i) screening for MHC Class II, CD45, epcam, UEA1 and/or Ly51 expression by cells of a mammalian biological sample, wherein expressing a phenotypic profile selected from:
   (i) MHC Class II^{lo}, CD45⁻ and epcam⁺;
   (ii) MHC Class II^{lo}, CD45⁻, UEA1^{lo} and Ly51^{lo}; or
   (iii) MHC Class II⁻, CD45⁻ and epcam⁺
   by a cell is indicative of a thymic epithelial progenitor cell; and
(ii) isolating said cell.

Still another aspect of the present invention provides a method for isolating a mammalian thymic cortical epithelial progenitor cell, said method comprising the steps of:
(i) screening for MHC Class II, CD45, UEA1 and/or Ly51 expression by cells of a mammalian biological sample, wherein expressing a phenotypic profile of MHC Class II^{lo}, CD45⁻, UEA1^{-/lo} and Ly51⁺ by a cell is indicative of a thymic cortical epithelial progenitor cell; and
(ii) isolating said cell.

Yet still another aspect of the present invention provides a method for isolating a mammalian thymic medullary epithelial progenitor cell, said method comprising the steps of:
(i) screening for MHC Class II, CD45, UEA1 and/or Ly51 expression by cells of a mammalian biological sample, wherein expressing a phenotypic profile of MHC Class II^{lo}, CD45⁻, UEA1⁺ and Ly51⁻ by a cell is indicative of a thymic medullary epithelial progenitor cell; and
(ii) isolating said cell.

In a further aspect, there is therefore provided a method for facilitating the proliferation and/or differentiation of a thymic epithelial progenitor cell of the present invention and/or a thymic epithelial progenitor cell which cell has been identified and/or isolated in accordance with the present invention, said method comprising contacting said progenitor cell, either in vitro or in vivo, with an effective amount of a stimulus for a time and under conditions sufficient to direct the proliferation or differentiation of said cell.

In still another aspect there is provided a thymic epithelial progenitor cell line, which cell line has been generated from a thymic epithelial progenitor cell of the present invention and/or a thymic epithelial progenitor cell which has been identified and/or isolated in accordance with the method of the present invention.

Yet another aspect provides a cellular population comprising a thymic epithelial progenitor cell of the present invention and/or a thymic epithelial progenitor cell which has been identified and/or isolated in accordance with the methods of the present invention, or cells differentiated therefrom.

Still another aspect provides a composition comprising a thymic epithelial progenitor cell of the present invention and/or a thymic epithelial progenitor cell which has been identified and/or isolated in accordance with the methods of the present invention, or cells differentiated therefrom, together with a pharmaceutically acceptable carrier or excipient.

In still another aspect there is provided a tissue aggregate, which tissue aggregate comprises a thymic epithelial progenitor cell of the present invention and/or a thymic epithelial progenitor cell which has been identified and/or isolated in accordance with the methods of the present invention, or cells differentiated therefrom.

In yet still another aspect there is provided isolated thymic tissue, which thymic tissue comprises a thymic epithelial progenitor cell of the present invention and/or a thymic epithelial progenitor cell which has been identified and/or isolated in accordance with the methods of the present invention, or cells differentiated therefrom.

In still yet another aspect there is provided an organoid, which organoid comprises a thymic epithelial progenitor cell of the present invention and/or a thymic epithelial progenitor cell which has been identified and/or isolated in accordance with the methods of the present invention of cells differentiated therefrom.

In another aspect there is therefore provided a method of generating a T cell, said method comprising co-culturing a T cell precursor with a thymic epithelial progenitor cell as hereinbefore defined and/or a cell differentiated therefrom for a time and under conditions sufficient to induce T cell precursor maturation.

There is also provided a method of generating a T cell in a mammal, said method comprising administering to said mammal a thymic epithelial progenitor cell of the present invention and/or a thymic epithelial progenitor cell which has been identified and/or isolated in accordance with the methods of the present invention and/or a cell differentiated therefrom, under conditions sufficient to induce T cell precursor maturation.

Another aspect of the present invention is directed to a method of therapeutically and/or prophylactically treating a condition in a mammal, which condition is characterised by aberrant or otherwise unwanted thymic epithelial cell structure or functioning, said method comprising administering to said mammal an effective number of the thymic epithelial progenitor cells of the present invention and/or thymic epithelial progenitor cells which have been identified and/or isolated in accordance with the methods of the present invention, or cells differentiated therefrom for a time and under conditions sufficient to induce proliferation or differentiation or to otherwise induce thymic formation.

The present invention provides a method for inducing tolerance to an antigen in a mammal, said method comprising reducing, ablating or otherwise downregulating the functionality of peripheral T cells in said mammal and administering to said mammal an effective number of thymic epithelial progenitor cells of the present invention and/or thymic epithelial progenitor cells which have been identified and/or isolated in accordance with the method of the present invention, or cells differentiated therefrom, which cells express at least one epitope of the subject antigen.

In still another aspect, there is provided a method of therapeutically or prophylactically inducing graft tolerance in a mammal, said method comprising reducing, ablating or otherwise downregulating the functionality of peripheral T cells in said mammal and administering to said mammal an effective number of thymic epithelial progenitor cells and/or thymic epithelial progenitor cells which have been identified and/or isolated in accordance with the method of the present invention, or cells differentiated therefrom, which cells express at least one MHC molecule of said graft.

In still yet another aspect there is provided a method of therapeutically or prophylactically treating graft rejection, said method comprising reducing, ablating or otherwise downregulating the functionality of peripheral T cells in said mammal and administering to said mammal an effective number of thymic epithelial progenitor cells and/or thymic epithelial progenitor cells which have been identified and/or isolated in accordance with the method of the present invention, or cells differentiated therefrom, which cells express at least one MHC molecule of said graft.

In another aspect, there is provided a method of therapeutically or prophylactically treating an autoimmune condition in a mammal, said method comprising reducing, ablating or otherwise downregulating the functionality of autoreactive peripheral T cells in said mammal and administering to said mammal an effective number of thymic epithelial progenitor cells of the present invention and/or thymic epithelial progenitor cells which have been identified and/or isolated in accordance with the method of the present invention, or cells differentiated therefrom, which cells express at least one epitope of the autoantigen to which said autoimmune condition is directed.

In yet another aspect, there is provided a method of therapeutically or prophylactically treating a hypersensitivity condition in a mammal, said method comprising reducing, ablating or otherwise downregulating the functionality of antigen reactive peripheral T cells in said mammal and administering to said mammal an effective number of thymic epithelial progenitor cells of the present invention and/or thymic epithelial progenitor cells which have been identified and/or isolated in accordance with the method of the present invention, or cells differentiated therefrom, which cells express at least one epitope of the antigen to which said hypersensitivity condition is directed.

There is provided a method of treating a mammal, said method comprising administering to said mammal a population of thymic epithelial progenitor cells of the present invention and/or thymic epithelial progenitor cells which have been identified and/or isolated in accordance with the method of the present invention, or cells differentiated therefrom, which cells express a protein or gene of interest.

According to yet another aspect of the present invention, there is provided a method of assessing the effect of a treatment or culture regime on the phenotypic state of the thymic epithelial progenitor cell of the present invention and/or thymic epithelial progenitor cells which have been identified and/or isolated in accordance with the method of the present invention, or cells differentiated therefrom, said method comprising subjecting said cells to said treatment regime and screening for an altered functional or phenotypic state.

The present invention also provides the epithelial progenitor cells of the present invention, or cells differentiated therefrom, for use in medicine.

The present invention additionally provides the thymic epithelial progenitor cells of the present invention or cells differentiated therefrom for use in treating a condition in a mammal, which condition is characterised by aberrant or otherwise unwanted thymic epithelial cell structure.

There is also provided the thymic epithelial progenitor cells of the present invention or cells differentiated therefrom for use in producing or regenerating thymus tissue in a mammal.

In another aspect there is provided the thymic epithelial progenitor cells of the present invention or cells differentiated therefrom for use in inducing the tolerance to an antigen in a mammal.

In still another aspect there is provided the thymic epithelial progenitor cells of the present invention or cells differentiated therefrom for use in therapeutically or prophylactically treating graft rejection, an autoimmune condition or an allergy or hypersensitivity condition.

In yet still another aspect there is provided the thymic epithelial progenitor cells of the present invention or cells differentiated therefrom for use in treating a mammal, which cells express a protein or gene of interest.

In a further aspect there is provided the thymic epithelial progenitor cells of the present invention or cells differentiated therefrom in the membrane of a medicament for the therapeutic or prophylactic treatment of a condition in a mammal, which condition is selected from:
(i) a condition characterised by aberrant or otherwise unwanted thymic epithelial cell structure;
(ii) graft rejection;
(iii) an autoimmune condition; or
(iv) an allergy or hypersensitivity condition.

In another further aspect there is provided thymic epithelial progenitor cells of the present invention or cells differentiated therefrom in the manufacture of a medicament for producing or regenerating the thymus or inducing tolerance to an antigen.

### BRIEF DESCRIPTION OF THE DRAWINGS

FIG. 1 is a graphical representation of total thymus cellularity pre- and post-treatment. Total thymus cellularity of untreated controls and various days post cessation of treatment are represented. CyclosporineA was administered to mice for 14 days, and cellularity measured at various time-points after cessation of treatment (A). Cyclophosphamide was administered for 2 days, and cellularity measured from 3 days after cessation of treatment (B). A single injection of dexamethasone was administered and thymic cellularity measured from 2 days after treatment (C) indicated as empty bars, with black bars indicating PBS controls. Cyclophosphamide and Dexamethasone treatment have the greatest impact on thymic cellularity and return to untreated levels by Day 14.

FIG. 2 is a graphical representation of changes in proportions of thymic epithelial cell subsets in CyclosporineA treated mice. Data shown refer to the number of days after cessation of treatment of CyclosporineA. Medullary epithelial cells (mTEC) are identified by a MHCII+UEA1+phenotype and cortical epithelial cells (cTEC) show a MHCII+UEA1−phenotype. mTEC are divided into MHCII high (mTEC hi) expressing subset and MHCII low (mTEC lo) expressing subset. cTEC are divided into cTEC hi and cTEC lo subsets (circles). Here, thymic epithelial precursor cells (TEPC) lie in the MHCII UEA1 lo (mTEC lo) subset. Proportions are represented as flow cytometric dot plots (A) or as bar graphs (B). The mTEC hi compartment shows an almost 4 fold reduction, resulting in a proportional increase in cTECs. Regeneration is initially evident in the cTEC compartment at Day 4 post-treatment with a proportional loss in mTEC lo cells. Putative MHCII lo UEA1 lo precursor cells may differentiate into cTEC lo cells, which then convert to cTEC hi cells in a linear fashion, or they may also contribute directly to cTEC hi cells. mTEC recovery is evident at Day 7 at the expense of mTEC lo cells. Recovery of mTEC lo subsets and a return to the normal untreated state is evident by Day 14.

FIG. 3 is a graphical representation of recovery in epithelial cell number after Cyclosporine A (CsA) treatment. There is a progressive decline in mTEC-lo cells from days 4 to 10 consistent with the progressive recovery of the mTEC-hi compartment. The mTEC-lo cells then recover to normal levels from days 10-14 by which time there is full recovery of mTEC cell numbers (A). An early increase in cTEC cell number (B) is evident from cessation of treatment to Day 4 post treatment followed by a return to homeostatic levels.

FIG. 4 is a graphical representation of proliferation of epithelial cell subsets post-Cyclosporine A treatment. Cell number of proliferating epithelial subsets (Ki67+) is represented as a bar graph (A). Flow cytometric dot plots (B) showing Ki67 staining are represented in the top row with the UEA1/MHCII profile of Ki67+ proliferating cells shown in the bottom row. The majority of proliferating cells in the untreated thymus are MHCII hi cells. Early proliferation of cTEC hi cells is evident on cessation of treatment with CsA (Day 0) although this is short-lived. Maximal proliferation of mTEC hi cells occurs from Day 7-10 with very few mTEC lo cells dividing. This is also seen in Figure (C) which shows that 23-35% of Ki67+ proliferating TECS are MHCII hi.

FIG. 5 is a graphical representation of aire+ve cells recover by Day 7 post CsA treatment. Aire positive cells are only found in the UEA1 hi mTEC compartment and begin reappearing from Day 7 after cessation of CsA treatment (A). However, the ratio of Aire negative to Aire positive mTECs, does not return to normal until around Day 28 (B) and this is reflected in the AIRE+ mTEC numbers (C).

FIG. 6 is a graphical representation of epithelial cell recovery after dexamethasone treatment. MHCII versus UEA1 dot plots in Figure (A) have PBS controls represented in the left panel and days after cessation of Dexamethasone treatment represented in the right panel. After an initial loss, the cTEC hi and mTEC hi cells recover at the expense of mTEC cells (A). The cTEC high cells appear at Day 7, either through upregulation of cTEC lo cells or direct differentiation from the mTEC lo cells. By Day 14, the mTEC hi subset has increased also at the expense of mTEC lo cells. A return to normal levels of mTEC hi and mTEC lo cells is evident by Day 28. These proportional changes in TEC subsets are represented as a bar graph in (B).

FIG. 7 is a graphical representation of epithelial cell numbers after Dexamethasone treatment. mTEC hi (A), mTEC lo (B) and cTEC (C) cell numbers from Day 2 post Dexamethasone treatment (dark bars) are shown in comparison to PBS controls (empty bars). Recovery of all subsets is evident from Day 14 post treatment.

FIG. 8 is a graphical representation of epithelial cell proportions after Cyclophosphamide treatment. Proportional changes in TEC subsets after Cyclophosphamide treatment are represented as flow cytometric dot plots (A). The loss of mTEC hi cells is still evident 3 days after cessation of Cyclophosphamide treatment, however by Day 7, both the cTEC and mTEC compartments show increased proportions at the expense of mTEC lo cells. It is unclear whether the movement into the cTEC compartment is via cTEC lo or directly to the cTEC hi phenotype. By Day 10, a further increase from cTEC lo to cTEC hi has occurred, and increase in mTEC hi cells is maximal by Day 14 followed by a replenishment of mTEC lo numbers by Day 28. The return to homeostatic proportions is apparent at Day 28. These mTEC proportional changes are reflected in the cell numbers of mTEC hi and mTEC lo populations (B).

FIG. 9 is a graphical representation of changes occur in epithelial cell proportions with age and castration-induced thymic regeneration. Thymic epithelial cells from young mice (6 weeks), aged mice (9 months) and at various timepoints during thymic regeneration after surgical castration were compared to sham-castration controls. UEA1 versus Ly51 dot plots are shown, with UEA1 hi cells representing mTECs and Ly51 hi cells representing cTECs. The UEA1 lo, Ly51 lo (TEClo) population is represented in the central circle. A proportional loss of mTECs occurs with age as well as a loss of cells in the cTEC hi subset (A). Regeneration of epithelial cell subsets after castration (B) is first evident as an increase in Ly51 hi cTECs at Day 7 with concomitant loss in TEClo cells. This is followed by an increase in mTECs at Day 10 and Day 14 at the expense of the TEClo subset. The Ly51 hi cTEC population is predominantly MHCII hi and the TEClo population is predominantly MHCII lo (see FIG. 11).

FIG. 10 is a graphical representation of TEClo cells show differential gene expression profiles. Preparation of thymic stromal cells require a gradual enzyme digestion of the thymic organ during which individual cells are released over time. UEA1 versus Ly51 cell profiles are represented here for the initial wash where free lymphocytes are released, followed by fractions 1-5 where stromal cells are gradually released (A). Ly51 hi cTECs are released mostly from the early digest fractions whilst the UEA1 hi mTECs are released predominantly in the later fractions. The TEClo population is present in all digested fractions. TEClo and cTEC fractions were sorted and analysed for expression of various genes known to be expressed in the thymus (B). The two populations show differential expression of these genes, confirming that they are a separate population of cells.

FIG. 11 is a graphical representation of MHC II expression on Ly51 hi/UEA1- and Ly51 lo/UEA1 lo cells. UEA1 versus Ly51 cell profiles of enzyme digested thymic tissue fractions 1-5, where stromal cells are gradually released, are represented here (panel A), showing UEA1hi/Ly51-mTECs, UEA1 lo/Ly51 lo TEClo cells and UEA1-/Ly51hi cTECs. The majority of UEA1 lo/Ly51 lo TEClo cells are MHC II lo expressing cells (panel B). The majority of Ly51hi cTECs are found in the earlier fractions and the majority of these are MHCII hi expressing cells (panel C).

FIG. 12 is a graphical representation depicting that CsA treatment causes transient thymic involution and loss of single positive thymocytes. A: thymus cellularity in mice following 2 weeks of CsA treatment. B: DP thymocyte. C: CD4 SP thymocytes. D: CD8 SP thymocytes. E: CD25 expression on CD45-stromal cells in untreated mice. Data shown as mean+SD. *=p<0.05 compared to untreated. CsA=cyclosporine A; Untr=untreated. Data is representative of 3 independent experiments. Data shown represents responses of 8 mice from 2 experiments.

FIG. 13 is a graphical representation depicting that regeneration of mTEC-hi occurs at the expense of mTEC-lo. A: Proportional alterations in TEC subsets after 14 days of CsA treatment B: TEC proportions. C: TEC numbers. Black bars=mTEC-hi, white=mTEC-lo, grey=cTEC-hi, hatched=cTEC-lo. Data represented as mean+SD. *=p<0.05 compared to the appropriate untreated TEC subset. Untr=untreated; CsA=cyclosporine A. Data shown represents responses of 8-10 mice from 2-3 experiments.

FIG. 14 is a graphical representation depicting the recovery of Aire. Proportion (A) and number (B) of TEC expressing Aire following 14 days of CsA treatment. C: ratio of Aire− mTEC-hi to Aire+ mTEC-hi cells. Data is shown as m+sd, *=p<0.05 compared to untreated. Aire gates were set according to isotype controls at each timepoint. Plots shown have been gated on TEC. Data shown represents responses of 8 mice from 2 experiments.

FIG. 15 is a graphical representation depicting that cell cycle analysis and variation of CsA treatment length show that mTEC-lo differentiate to mTEC-hi. A: Ki67+ TEC during thymic recovery after 14 days of CsA treatment. Black bars=mTEC-hi, white=mTEC-lo, grey=cTEC-hi, hatched=cTEC-lo. B: recovery of TEC subsets at day 7 post treatment, after a shortened (7 days) CsA treatment regime. Black bars=untreated, white bars=CsA treated. Data represented as mean+SD. *=p<0.05 compared to the relevant untreated TEC subset. Untr=untreated; CsA=cyclosporine A. Data shown represents responses of 8-10 mice from 2 experiments.

FIG. 16 is a graphical representation depicting that pTESC are Ly51-lo prior to differentiation. A: Alteration in Ly51 expression on mTEC-lo during thymic recovery post-CsA treatment. Unt=untreated; CsA=cyclosporine A; D=days post CsA cessation. B: In untreated mice, the TEC population expressing low levels of both IJEA1 and Ly51 (denoted as gate 2) expresses low levels of MHC class II and falls within the mTEC-lo gate. Gates 1 and 3 contain MHC class II-high mTEC and cTEC. Data shown represents responses of 8-10 mice from 2-3 experiments.

FIG. 17 is a graphical representation depicting that mTEC-lo progenitor cells lose MTS24 upon differentiation. A: MTS24 expression on CD45-stromal cells (dotplot) and on TEC subsets (histograms). MTS24 expression post-CsA treatment as a proportion of TEC subsets (B) and total MTS24+ cells (C). Black bars=mTEC-hi, white=mTEC-lo, grey=cTEC-hi, hatched=cTEC-lo. *p<0.05 compared to the relevant untreated TEC subset. Untr=untreated; CsA=cyclosporine A. Data shown represents responses of 8 mice from 2 experiments.

FIG. 18 is a graphical representation of thymus cellularity post-cyclophosphamide (A) and dexamethasone (B) treatment. Untr=untreated. Cyclo=cyclophosphamide. Dex=dexamethasone. Data represented as m+SD. *p<0.05 compared to untreated. Data shown represents responses of 8-10 mice from 3 experiments.

FIG. 19 is a graphical representation depicting that TEC recovery following cyclophosphamide or dexamethasone treatment. TEC proportions following cyclophosphamide treatment of male mice (A) or dexamethasone treatment of female mice (B). TEC numbers following cyclophosphamide treatment (C) or dexamethasone treatment (D). Black bars=mTEC-hi, white=mTEC-lo, grey=cTEC-hi, hatched=cTEC-lo. Cyclo=cyclophosphamide. Dex=dexamethasone. Data represented as M+SD. P<0.05 compared with the appropriate untreated TEC subset. Data shown represents responses of 8-10 mice from 3 experiments.

FIG. 20 is a graphical representation depicting that phenotypic analysis of mTEC-lo subset. A: CD80 expression on mTEC populations. B: Thymic distribution of keratin 5 (K5; red) and keratin 8 (K8; green) by immunohistology and flow cytometry. The dotted line surrounds a medullary islet. Flow cytometry gates set using isotype controls. C: p63 expression in TEC subsets shown relative to mTEC-lo (normalized to 1, shown by dotted line) by qPCR. *p<0.05 compared to mTEC-lo; m+SE. Data shown represents responses of 4-5 mice from 2-3 experiments.

FIG. 21 is a graphical representation of the differential expression of IL7, CCL19 and CCL25 in adult stromal cell subsets.

FIG. 22 is a schematic representation of the lentiviral backbone and bicistronic vector encoding MOG and eGFP.

FIG. 23 is a graphical representation of the co-expression of eGFP in medullary epithelial thymic stromal cells. A. Characteristic profile of MHC class II and eGFP expression (gated on CD45⁻ cells) in control and intrathymically injected mice. B. Characteristic profile of MHC II and UEA1 expression (gated on CD45⁻ cells). C. eGFP expression in control and intrathymically injected mice in medullary epithelial stromal cells based on the expression of UEA1 and MHC II. Cells were gated on CD45⁻ cells.

FIG. 24 is a graphical representation of the co-expression of eGFP in cortical epithelial thymic stromal cells. A. Characteristic profile of Ly51 and eGFP expression (gated on CD45⁻ cells) in control and intrathymically injected mice. B. Characteristic profile of MHC II and Ly51 expression (gated on CD45⁻ cells). C. eGFP expression in control and intrathymically injected mice in cortical epithelial stromal cells based on the expression of Ly51 and MHC II. Cells were gated on CD45⁻ cells.

### DETAILED DESCRIPTION OF THE INVENTION

The present invention is predicated, in part, on the identification and isolation of novel thymic epithelial progenitor subpopulations. Still further, there has been achieved the characterisation of these cellular populations in terms of the cell surface co-expression of MHC Class II, UEA 1 and Ly51. Accordingly, where it was not previously possible to engineer or otherwise repair thymic cortical and/or medullary tissue in vitro or in vivo, due to the absence of an isolated thymic epithelial progenitor cellular population from which differentiation could be effected, this finding has now facilitated the development of such methodology. Still further, phenotypic characterisation of the newly identified and isolated subpopulations has rendered it possible to efficiently and reliably detect and/or isolate these cells in a prospective manner. These findings therefore provide a means for the routine detection and isolation of thymic epithelial cellular subpopulations for use in vitro or in vivo, including for the therapeutic or prophylactic treatment of conditions characterised by thymic tissue damage or the in vitro assessment of the effectiveness and/or toxicity of potential treatment or culture regimes to which thymic epithelial tissue may be exposed. Also facilitated is a means for monitoring thymic epithelial cellular subpopulations either in vitro or in vivo.

Accordingly, in one aspect there is provided an isolated mammalian thymic epithelial progenitor cell expressing an MHCII^{lo} phenotypic profile or mutant or variant thereof.

Reference to “mammal” or “mammalian” should be understood to include reference to a mammal such as but not limited to human, primate, livestock (animal (eg. sheep, cow, horse, donkey, pig), companion animal (eg. dog, cat), laboratory test animal (eg. mouse, rabbit, rat, guinea pig, hamster), captive wild animal (eg. fox, deer). Preferably the mammal is a human or primate. Most preferably the mammal is a human.

Reference to “thymic epithelial progenitor cellular population” should be understood as a reference to a population of epithelial progenitor cells which are capable of differentiating to an epithelial cell which would be found in the thymic microenvironment in nature. This progenitor cell may be found either in the thymic microenvironment or outside the thymic microenvironment. It should also be understood that reference to a thymic cortical or medullary progenitor cell is not intended as a reference to a progenitor cell which is necessarily found in the cortex or medulla of the thymus. Rather, it is a progenitor cell which may be found in any region of the thymus, or indeed any other tissue or ex vivo, but which can differentiate to a mature epithelial cell which would become localised to the thymic cortex or medulla, respectively, in a subject.

The epithelial nature of the subject cell is characterised by a CD45⁻/epcam⁺ cell surface antigen phenotype. To this end, it should be understood that to the extent that it is not specified, reference to a thymic “epithelial” cell is a reference to a cell which is characterised by a CD45⁻/epcam⁺ phenotype, this being a characteristic epithelial phenotype. However, it should be understood that characterising the epithelial nature of these cells can be achieved by screening for any suitable epithelial phenotypic or morphological feature, of which CD45⁻/epcam⁺ is one suitable means.

The present invention therefore more particularly provides an isolated mammalian thymic epithelial progenitor cell expressing a phenotypic profile of CD45⁻, epcam⁺ and MHC Class II^{lo} or mutant or variant thereof.

By “progenitor” is meant that the cell is not fully differentiated but requires further differentiation to achieve maturation. Such cells are often also sometimes referred to as “precursor” cells, “multipotent” cells, “pluripotent” cells or “stem” cells (although the latter two terms are generally reserved for cells which exhibit extensive potentiality). The subject progenitor cell may be one which exhibits multipotentiality, for example is a progenitor which can be induced to differentiate down either the cortical or medullary thymic lineages, or it may already be committed to one of these two lineages. However, despite this initial level of commitment, the subject cell is nevertheless still a “progenitor” on the basis that it is not fully differentiated. The use of the term “progenitor” should not be understood as a limitation on the maturity/immaturity of the subject cell relative to that which might be implied by the use of the terms “stem cell”, “multipotent cell”, “pluripotent cell” or other such term.

Reference to the “thymic microenvironment” should be understood as a reference to all regions of the thymus including the thymic lobes and lobules, the thymic stroma (including both the cortical and medullary regions of each lobule), the cortico-medullary junction region, the capsule and subcapsular-perivascular regions, the lymphocytic cells and the non-lymphocytic cells such as the stromal cells, macrophages, dendritic cells, Hassells corpuscles and the like. Without limiting the present invention to any one theory or mode of action, the thymus is a bilobed glandular organ located in the upper anterior thorax. The thymus is encapsulated and divided into lobules, each lobule consisting of an outer cortical region and an inner medullary region. Together with connective tissue, the cortical and medullary epithelium form the thymic stroma. The thymus is colonised by cells of haemopoietic origin including thymocyte precursors and myeloid cells which form the macrophage and dendritic cell populations of the thymus. Accordingly, it should be understood that reference to “thymic microenvironment” includes reference to both thymic tissue which has differentiated from the germ layer tissue of the third pharyngeal pouch and third bronchial cleft and thymic cellular populations which have colonised either the thymic anlage or the thymus, such as myeloid or lymphoid cells of bone marrow or liver origin.

The subject cellular population may be a single cell, single cell suspension or a cell aggregate, such as a tissue or a part thereof, which has been freshly isolated from an individual (such as an individual who may be the subject of treatment) or it may have been sourced from a non-fresh source, such as from a culture (for example, where cell numbers were expanded and/or the cells were cultured so as to render them receptive to differentiative signals) or a frozen stock of cells (for example, an established cell line), which had been isolated at some earlier time point either from an individual or from another source. It should also be understood that the subject cells may have undergone some other form of treatment or manipulation, such as but not limited to enrichment or purification, modification of cell cycle status, molecular transformation or the formation of a cell line. Accordingly, the subject cell may be a primary cell or a secondary cell. A primary cell is one which has been freshly isolated from an individual. A secondary cell is one which, following its isolation, has undergone some form of in vitro manipulation such as the preparation of a cell line.

As detailed hereinbefore, the present invention is predicated on the identification and phenotypic characterisation of previously unknown cellular populations which form part of the cellular hierarchy which is involved in thymus development. Phenotypic characterisation of this cellular population has revealed that it is CD45⁻, epcam⁺ and MHC Class II^{lo}. It should be understood that although this cell is capable of differentiation along a thymic epithelial lineage, it is not irreversibly committed in this regard and could, in the presence of appropriate signals, differentiate along a non-thymic epithelial lineage. In addition to this progenitor cell population, there has also been identified a thymic epithelial progenitor population which is characterised by MHC Class II^{lo}, UEA1^{lo} and Ly51^{lo} expression (this cell may also be interchangeably referred to as a multipotent cell or stem cell). It is thought that this cellular population may be the common progenitor of two further and more differentiated progenitor thymic epithelial cell populations which have been herein identified, these being:

(i) MHC Class II^{lo}, UEA1⁺ and Ly51⁻ thymic medullary epithelial progenitor cell;
(ii) MHC Class II^{lo}, UEA1^{-/lo} and Ly51⁺ thymic cortical epithelial progenitor cell.

These latter cellular populations are believed to be committed to differentiating along the medullary and cortical thymic epithelial lineages, respectively, although this commitment is not necessarily irreversible. Accordingly, there have been identified four novel thymic progenitor cell populations which are thought to be reflective of progressively more differentiated stages of development but which, although demonstrating some level of differentiative commitment, are nevertheless progenitor/precursor cells which require the receipt of appropriate differentiative signals to effect their differentiation to mature, functional cortical or medullary thymic epithelial cells. In a related aspect, there has also been identified a thymic epithelial progenitor population exhibiting the phenotypic cell surface marker profile epcam⁺, CD45⁻ and MHC Class II⁻. Without limiting the present invention in any way, it is thought that this progenitor population may be even more immature than the CD45⁻, epcam⁺ and MHC Class II^{lo} population and may also therefore be described as a stem cell, multipotent cell or pluripotent cell. It should also be understood that UEA1 and Ly51 are thymic markers characteristic of medullary and cortical cells, respectively. Accordingly, it is expected that other markers which are characteristic of these cellular subpopulations may be used instead of one or both of UEA1 and Ly51.

Reference to a “mutant or variant” of the subject cellular population should be understood as a reference to a cell which is derived from the cellular population but exhibits at least one difference at the phenotypic or functional level. For example, the mutant or variant may have altered the expression of its cell surface markers or some aspect of its functionality subsequently to initial isolation. Such changes can occur either spontaneously (as exemplified by the spontaneous upregulation or downregulation of cell surface markers which can occur subsequently to in vitro culture or spontaneous transformation) or as a result of a directed manipulation, such as would occur if a cell was deliberately transformed (for example, in order to effect the creation of a cell line) or transfected (for example to effect the expression of a particular gene or marker).

It should be understood that the thymic epithelial cellular populations of the present invention may exhibit some variation in differentiative status within a single phenotypic profile. That is, within a single phenotypic profile, although the cells comprising that profile may substantially exhibit similar phenotypic and/or functional characteristics, there may nevertheless exhibit some differences. This may be apparent, for example, in terms of differences in the transcriptome profile or cell surface marker expression (other than the markers defined herein) of the cells which comprise the phenotypic profile in issue. For example, the MHC Class II^{lo}, CD45⁻, epcam⁺ cells may not represent a highly specific and discrete stage, but may be characterised by a number of discrete cellular subpopulations which reflect a transition or phase if one were to compare cells which have differentiated into this stage versus cells which are on the cusp of maturing out of this stage. This is typically characterised, for example, by the onset of a sequential series of changes to gene expression, two or more of which are required to occur before the characteristic phenotypic profile defined herein is changed. Accordingly, the existence of cellular subpopulations within a single phenotypic profile of the present invention is encompassed.

It would be appreciated that the phenotypic characterisation of the novel cellular populations of the present invention is a significant development since the identification of unique marker profiles in the context of the thymic epithelial cell hierarchy has remained elusive. Without limiting the present invention to any one theory or mode of action:
(i) MHC Class II antigens (or “MHC II antigens”) are major histocompatibility antigens, which are expressed on the surface of antigen-presenting cells, including dendritic cells, macrophages, B lymphocytes and certain epithelial cell types (e.g. cortical and medullary thymic epithelial cells). MHC molecules are essential for the presentation of antigenic peptides to CD4 T lymphocytes. Its expression is up-regulated on activated antigen-presenting cells. In certain contexts (e.g. an inflammatory context), MHC Class II is expressed by other cell types (e.g. endothelial cells, islet beta cells) that do not usually express MHC Class II. MHC Class II is expressed on the cell surface as a heterodimer consisting of an alpha chain and a beta chain. The tertiary structure of the molecule is such that a peptide-binding ‘groove’ or ‘cleft’ is formed between the α1 and β1 domains of the alpha and beta chains respectively. The region of the groove is highly polymorphic, resulting in a high degree of allelic variation between MHC Class II molecules within outbred populations. Conventionally, the peptides are derived from extracellular proteins, which have been endocytosed by the cell, and are usually at least around 15 amino acids in length. It is within the endocytic compartment of a cell that the peptides are loaded into the groove, forming an MHC::peptide complex, which is subsequently transported to the cell surface. The MHC::peptide complex is recognised by a T cell receptor on the surface of a T lymphocyte. In humans, MHC Class II antigens are also called Human Leukocyte Antigens (HLA), examples of which are HLA-DM, HLA-DO, HLA-DP, HLA-DQ and HLA-DR. Examples of MHC Class II antigens in inbred laboratory mouse strains are IA and IE.
(ii) UEA-1 or Ulex europaeus agglutinin-1, is a lectin molecule (i.e. a carbohydrate-binding protein or glycoprotein), which binds with high affinity to the surface of endothelial cells, (e.g. human ventricular endothelial cells or HUVEC), and intestinal M cells, all of which express the UEA-1 ligand. UEA-1 is often used to stain stromal cells in the thymic medulla (i.e. those cells not of haematopoietic origin).
(iii) Ly-51, also known as 6C3 and BP-1, is a type II disulfide-linked homodimeric transmembrane glycoprotein, expressed at high levels on bone marrow stromal cell lines and on a variety of mouse tissues known to possess aminopeptidase activity. Subsets of normal pre-B and B cells express low levels of Ly-51, which is rapidly upregulated on pre-B cells in the presence of IL-7. Ly-51 is also expressed by stromal cells in the thymic cortex. The Ly-51-specific antibody clone, 6C3 was generated by immunising rats with the mouse Pre-B lymphoma cell line L1-2 plus Abelson murine leukemia virus-specific cytotoxic T-cell clones.
(iv) Epcam, or epithelial cell adhesion molecule, is also known as human epithelial antigen-125 (HEA-125), CD326, GA733-2, KSA, and 17-1A antigen. It is a 40 kDa transmembrane glycoprotein involved in cell adhesion, is broadly expressed on the basolateral surface of carcinoma and epithelial cells but is not found on melanoma, neuroblastoma, sarcoma, lymphoma, leukemia cells, or normal fibroblasts.
(v) CD45 is a protein tyrosine phosphatase, essential for signalling through the T cell receptor. It is also known as the leukocyte common antigen (L-CA) or T200. Variants of CD45 are CD45-RO, RA, rBACE1 and RC.

In the context of the present invention, it should be understood that reference to “MHC Class II”, “CD45”, “UEA1”, “Ly51” and “epcam” is a reference to all forms of these molecules and to functional fragments, mutants or variants thereof. It should also be understood to include reference to any isoform which may arise from alternative splicing of “MHC Class II”, “CD45”, “UEA1”, “Ly51” and “epcam” mRNA or isomeric or polymorphic forms of these molecules.

Reference to “phenotypic profile” should be understood as a reference to the presence or absence or level of the transcription of the genes encoding the subject markers and/or the cell surface expression of the expression product translated therefrom. It should be appreciated that although most cells falling within the scope of the claimed thymic epithelial cellular populations will be characterised by the presence or absence of the subject marker as a cell surface anchored expression product, some cells falling within the defined populations may exhibit changes only at the transcriptome level, such as when the transcription of a given marker has been upregulated but may not yet have resulted in a cell surface anchored expression product. In general, cells which progress to a new differentiative stage will transiently exhibit gene expression changes which are not yet evident in the context of changes to levels of an expression product. However, these cells nevertheless fall within the scope of the claimed cellular populations.

It should also be appreciated that although the thymic epithelial cell populations of the present invention are characterised by the defined phenotypic profiles, these cells will express a range of other intracellular and/or cell surface markers which are not relevant in terms of phenotypically characterising the cellular population of interest. Still further, to the extent that a given thymic epithelial cellular population of the present invention comprises a range of subpopulations, these subpopulations may exhibit variations in the expression of intracellular or cell surface markers other than those of the profiles defined herein.

As detailed hereinbefore, although some of the subject cell surface markers are defined by reference to the presence or absence of the marker on the cell surface, the expression of MHC Class II, UEA1 and Ly51 is defined in certain embodiments by reference to the level of expression, specifically low level expression (herein referred to as “MHC Class II^{lo}”). Reference to “MHC Class II^{lo}” should be understood as a reference to a level of expression which, when measured by FACS, corresponds to the middle boundary of the three broad categories of MHC Class II intensity staining which are observed. That is, the localisation of the MHC Class II^{lo} population will be evident relative to the cTEC and mTEC populations and can be routinely determined by the person of skill in the art. For example, MHC Class II^{lo} expression would be typically characterised by Log 2 to Log 4 (i.e. 200 to 400) where the other two classes of MHC Class II expression would typically be observed at 0 to Log 1 and above Log 4 (i.e. above 400—MHC Class II^{hi}). However, whereas this is observed when one performs the analysis using digital software (such as Diva software), where analogue software is utilised (eg. Cell Quest), the MHC II^{lo} calls would be observed to fall in the range Log 1 to Log 3. A corresponding meaning shall be taken to apply to the terms “UEA1^{lo}” and “Ly51^{lo}”. To this end, to the extent that the thymic cortical and medullary epithelial progenitor cell populations are defined as Ly51⁺ and UEA1⁺, respectively, it should be understood that this level of expression is higher than the “lo” level of expression hereinbefore defined.

The present invention therefore provides an isolated mammalian thymic epithelial progenitor cell expressing a phenotypic profile selected from:

### (i) MHC Class II^{lo};

### (ii) MHC Class II^{lo}, UEA1^{lo} and Ly51^{lo}; or

(iii) MHC Class II⁻
or mutant or variant thereof.

The present invention more particularly provides an isolated mammalian thymic epithelial progenitor cell expressing a phenotypic profile selected from:

(i) MHC Class II^{lo}, CD45⁻ and epcam⁺;

### (ii) MHC Class II^{lo}, CD45⁻, UEA1^{lo} and Ly51^{lo}; or

(iii) MHC Class II⁻, CD45⁻ and epcam⁺
or mutant or variant thereof.

Said progenitor cell preferably exhibits potentiality for cortical and/or medullary thymic epithelial cell differentiation.

In another aspect, there is provided an isolated thymic cortical progenitor cell expressing a phenotypic profile of MHC Class II^{lo}, UEA1^{-/lo} and Ly51⁺ or mutant or variant thereof.

In still another aspect, there is provided an isolated thymic medullary progenitor cell expressing a phenotypic profile of MHC Class II^{lo}, UEA1⁺ and Ly51⁻ or mutant or variant thereof.

Preferably, said cortical and medullary cells are CD45⁻.

More preferably, said mammal is a human.

In a related aspect, the phenotypic characterisation of the cellular populations identified herein has now facilitated the development of methodology to efficiently and reliably detect and/or isolate these thymic epithelial progenitor populations. These findings therefore provide a means for the routine detection and isolation of these cellular populations from any available tissue source for use in vitro or in vivo, including for the therapeutic or prophylactic treatment of conditions characterised by aberrancies in thymic structure or function. This development is particularly useful since it now provides means for prospectively isolating the subject thymic epithelial cellular populations.

Accordingly, another aspect of the present invention is directed to a method for identifying a mammalian thymic epithelial progenitor cell, said method comprising screening for MHC Class II, CD45, epcam, UEA1 and/or Ly51 cellular expression in a mammal or in a biological sample derived from said mammal wherein expression of a phenotypic profile selected from:

(i) MHC Class II^{lo}, CD45⁻ and epcam⁺;

### (ii) MHC Class II^{lo}, CD45⁻, UEA1^{lo} and Ly51^{l0}; or

(iii) MHC Class II⁻, CD45⁻ and epcam⁺
is indicative of a thymic epithelial progenitor cell.

Preferably, said progenitor cell exhibits potentiality for cortical and/or medullary thymic epithelial cell differentiation.

In another aspect, the present invention is directed to a method for identifying a mammalian thymic cortical progenitor cell, said method comprising screening for MHC Class II, CD45, UEA1 and Ly51 cellular expression in a mammal or in a biological sample derived from said mammal wherein expression of the phenotypic profile of MHC Class II^{lo}, CD45⁻, UEA1^{-/lo} and Ly51⁺ is indicative of a thymic cortical progenitor cell.

In still another aspect, the present invention is directed to a method for identifying a mammalian thymic medullary progenitor cell, said method comprising screening for MHC Class II, CD45, UEA1 and Ly51 cellular expression in a mammal or in a biological sample derived from said mammal wherein expression of the phenotypic profile of MHC Class II^{lo}, CD45⁻, UEA1⁺ and Ly51⁻ is indicative of a thymic medullary progenitor.

It should be understood that the present invention is not intended to necessarily be limited to screening for the specified markers. For example, one may seek to simultaneously screen for other markers of interest, such as CD45 and epcam, in order to enable confirmation of the epithelial nature of the subject cell. However, any other suitable method for confirming the epithelial nature of the cell may be alternatively utilised. Yet other markers of interest may also be screened for, such as other epithelial markers or cortical or medullary specific markers.

To the extent that the method of the present invention is performed in vitro, on an isolated tissue sample, rather than as an in vivo based screen, reference to “biological sample” or “tissue sample” (these terms being used interchangeably) should be understood as a reference to any sample of biological material derived from an animal such as, but not limited to, cellular material (eg. tissue aspirate), tissue biopsy specimens (eg. thymic biopsies), surgical specimens or biological fluids (e.g. adult blood or cord blood). The biological sample which is tested according to the method of the present invention may be tested directly or may require some form of treatment prior to testing. For example, a biopsy or surgical sample may require homogenisation or other form of cellular dispersion prior to testing or it may require sectioning for in situ testing. Further, to the extent that the biological sample is not in liquid form, (if such form is required for testing) it may require the addition of a reagent, such as a buffer, to mobilise the sample. Alternatively, it may require some other form of pretreatment such as heparinisation, where the sample includes a blood component, in order to prevent clotting.

The biological sample may be directly tested (eg. in the context of cell surface MHC Class II, UEA1, Ly51, epcam and/or CD45 expression) or else all or some of the nucleic acid material present in the biological sample may be isolated prior to testing (eg. to assess MHC Class II, UEA1, Ly51, epcam and/or CD45 mRNA expression). Please note that although the latter form of testing is not the preferred form of testing, due to its inherently more complex nature than cell surface expression analysis, there may nevertheless occur situations in which it is necessary or desirable to perform nucleic acid based analysis. Accordingly, the present invention should be understood to extend to such analyses. In yet another example, the sample may be partially purified or otherwise enriched prior to analysis. For example, to the extent that a biological sample comprises a very diverse cell population, it may be desirable to select out a sub-population of particular interest. It is within the scope of the present invention for the target cell population or molecules derived therefrom to be pretreated prior to testing, for example inactivation of live virus. It should also be understood that the biological sample may be freshly harvested or it may have been stored (for example by freezing) prior to testing or otherwise treated prior to testing (such as by undergoing culturing, for example to expand or stabilise the thymic progenitor cell population).

The choice of what type of sample is most suitable for testing in accordance with the method disclosed herein will be dependent on the nature of the situation, such as the nature of the condition being monitored.

As hereinbefore described the subject tissue (which includes reference to “cells”) may have been freshly isolated from an individual (such as an individual who may be the subject of treatment) or it may have been sourced from a non-fresh source, such as from a culture (for example, where cell numbers were expanded and/or the cells were cultured so as to render them receptive to differentiative signals) or a frozen stock of cells (for example, an established cell line), which had been isolated at some earlier time point either from an individual or from another source.

As detailed above, the thymic epithelial progenitor cells of the present invention are characterised by the co-expression of specific profiles of MHC Class II, UEA1, Ly51, epcam and/or CD45. This is a significant development since the cell surface phenotyping of thymic epithelial progenitor cells, in terms of identifying the existence and profiles of specific subpopulations, has remained elusive. For this reason, the present determination that the specified MHC Class II, UEA1, Ly51, epcam and/or CD45 profiles represent unique markers of these newly identified classes of cells is an extremely significant determination and enables prospective isolation of these cells.

Means of screening for MHC Class II, UEA1, Ly51, epcam and/or CD45 expression in a mammal, or biological sample derived therefrom, can be achieved by any suitable method, which would be well known to the person of skill in the art, such as but not limited to:
(i) Measurement of altered MHC Class II, UEA1, Ly51, epcam and/or CD45 in a suitable biological sample, either qualitatively or quantitatively, for example by immunoassay, utilising immunointeractive molecules such as antibodies to detect MHC Class II, UEA1, Ly51, epcam and/or CD45 and/or any other additional molecule of interest.

In one example, one may seek to detect MHC Class II, UEA1, Ly51, epcam and/or CD45 immunointeractive molecule complex formation. For example, an antibody, having a reporter molecule directly associated therewith, may be utilized in immunoassays. Such immunoassays include but are not limited to radioimmunoassays (RIAs), FACS, magnetic bead sorting, enzyme-linked immunosorbent assays (ELISAs), tissue section staining and immunochromatographic techniques (ICTs), Western blotting which are well known to those of skill in the art. For example, reference may be made to “Current Protocols in Immunology”, 1994 which discloses a variety of immunoassays which may be used in accordance with the present invention. Immunoassays may include competitive assays. It will be understood that the present invention encompasses qualitative and quantitative immunoassays.

Suitable immunoassay techniques are described, for example, in U.S. Pat. Nos. 4,016,043, 4,424,279 and 4,018,653. These include both single-site and two-site assays of the non-competitive types, as well as the traditional competitive binding assays. These assays also include direct binding of a labelled antigen-binding molecule to a target antigen or indirect binding of a labelled reporter molecule to a first antibody. The antigen in this case is MHC Class II, UEA1, Ly51, epcam and/or CD45.

From the foregoing, it will be appreciated that the reporter molecule associated with the antibody may include the following: —
(a) direct attachment of the reporter molecule to the first antibody;
(b) indirect attachment of the reporter molecule to the first antibody; i.e., attachment of the reporter molecule to another assay reagent, such as a second antibody, which subsequently binds to the first antibody; and
(c) attachment to a subsequent reaction product of the antibody.

The reporter molecule may be selected from a group including a chromogen, a catalyst, an enzyme, a fluorochrome, a chemiluminescent molecule, a paramagnetic ion, a lanthanide ion such as Europium (Eu³⁴), a radioisotope including other nuclear tags and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in U.S. Pat. No. 4,366,241, U.S. Pat. No. 4,843,000, and U.S. Pat. No. 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), cy-chrome, APC (allophycocyanin) tetramethylrhodamine isothiocyanate (TRITC), R-Phycoerythrin (RPE), and Texas Red. Other exemplary fluorochromes include those discussed by Dower et al., International Publication No. WO 93/06121. Reference also may be made to the fluorochromes described in U.S. Pat. Nos. 5,573,909 (Singer et al), 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodate. As will be readily recognized, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex, allowed to bind, and then the excess reagent washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of MHC Class II, UEA1, Ly51, epcam and/or CD45 which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art and are particularly useful for the present method. However, other reporter molecules, such as radioisotope, chemiluminescent or bioluminescent molecules may also be employed.
(ii) In vivo detection of MHC Class II, UEA1, Ly51, epcam and/or CD45. Molecular Imaging may be used following administration of imaging probes or reagents capable of disclosing altered levels of the MHC Class II, UEA1, Ly51, epcam and/or CD45 cells in the tissue.

Molecular imaging (Moore et al., Nature Medicine, 6:351-355, 2000) is the in vivo imaging of molecular expression that correlates with the macro-features currently visualized using “classical” diagnostic imaging techniques such as X-Ray, computed tomography (CT), MRI, Positron Emission Tomography (PET) or endoscopy.
(iii) Detection of upregulation or downregulation of mRNA expression in the cells by Fluorescent In Situ Hybridization (FISH), real time PCR, or in extracts from the cells by technologies such as Quantitative Reverse Transcriptase Polymerase Chain Reaction (QRTPCR) or Flow cytometric qualification of competitive RT-PCR products (Wedemeyer et al., Clinical Chemistry 48(9):1398-1405, 2002) or array technologies. It should be understood that although this is not necessarily the preferred method for performing the method of the invention, it is nevertheless a method which may be suitable for use in some circumstances and is therefore encompassed by the scope of the present invention.

For example, a labelled polynucleotide encoding MHC Class II, UEA1, Ly51, epcam and/or CD45 may be utilized as a probe in a Northern blot of an RNA extract obtained from a cellular population. Preferably, a nucleic acid extract from the mammal is utilized in concert with oligonucleotide primers corresponding to sense and antisense sequences of a polynucleotide encoding MHC Class II, UEA1, Ly51, epcam and/or CD45, or flanking sequences thereof, in a nucleic acid amplification reaction such as RT PCR, real time PCR or SAGE. A variety of automated solid-phase detection techniques are also appropriate. For example, a very large scale immobilized primer arrays (VLSIPS™) are used for the detection of nucleic acids as, for example, described by Fodor et al., 1991 (Science 251(4995):767-73) and Kazal et al., 1996. The above genetic techniques are well known to persons skilled in the art.

For example, to detect MHC Class II, UEA1, Ly51, epcam and/or CD45 encoding RNA transcripts, RNA is isolated from a cellular sample suspected of containing MHC Class II, UEA1, Ly51, epcam and/or CD45 RNA, e.g. total RNA isolated from a human compact bone sample. RNA can be isolated by methods known in the art, e.g. using TRIZOL™ reagent (GIBCO-BRL/Life Technologies, Gaithersburg, Md.). Oligo-dT, or random-sequence oligonucleotides, as well as sequence-specific oligonucleotides can be employed as a primer in a reverse transcriptase reaction to prepare first-strand cDNAs from the isolated RNA. Resultant first-strand cDNAs are then amplified with sequence-specific oligonucleotides in PCR reactions to yield an amplified product.

“Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers. These primers will be identical or similar in sequence to opposite strands of the template to be amplified. PCR can be used to amplify specific RNA sequences and cDNA transcribed from total cellular RNA. See generally Mullis et al., 1987, Methods Enzymol 155:335-50; Erlich (1989) J Clin Immunol 9(6):437-47. Thus, amplification of specific nucleic acid sequences by PCR relies upon oligonucleotides or “primers” having conserved nucleotide sequences wherein the conserved sequences are deduced from alignments of related gene or protein sequences, e.g. a sequence comparison of mammalian MHC Class II, UEA1, Ly51, epcam and/or CD45 genes. For example, one primer is prepared which is predicted to anneal to the antisense strand and another primer prepared which is predicted to anneal to the sense strand of a cDNA molecule which encodes MHC Class II, UEA1, Ly51, epcam and/or CD45.

To detect the amplified product, the reaction mixture is typically subjected to agarose gel electrophoresis or other convenient separation technique and the relative presence of the MHC Class II, UEA1, Ly51, epcam and/or CD45 specific amplified DNA detected. For example, MHC Class II, UEA1, Ly51, epcam and/or CD45 in amplified DNA may be detected using Southern hybridization with a specific oligonucleotide probe or comparing is electrophoretic mobility with DNA standards of known molecular weight. Isolation, purification and characterization of the amplified MHC Class II, UEA1, Ly51, epcam and/or CD45 DNA may be accomplished by excising or eluting the fragment from the gel (for example, see references Lawn et al., 1981; Goeddel et al., 1980), cloning the amplified product into a cloning site of a suitable vector, such as the pCRII vector (Invitrogen), sequencing the cloned insert and comparing the DNA sequence to the known sequence of MHC Class II, UEA1, Ly51, epcam and/or CD45. The relative amounts of MHC Class II, UEA1, Ly51, epcam and/or CD45 mRNA and cDNA can then be determined.
(iv) Determining altered protein expression based on any suitable functional test, enzymatic test or immunological test in addition to those detailed, above.

As detailed hereinbefore, it should be understood that in the context of the method of the present invention one may design the diagnostic test such that only MHC Class II, UEA1, Ly51, epcam and/or CD45 form the subject of testing. However, it is to be expected that in some situations one might also analyse other functional or phenotypic features such as, preferably, other differentiation or cell class specific markers.

In a related aspect, there is provided a method for identifying a mammalian thymic epithelial progenitor cell, said method comprising screening for MHC Class II, UEA1, Ly51, epcam and/or CD45 expression by the cells of a biological sample wherein expression of a phenotypic profile selected from:
(i) MHC Class II^{lo}, CD45⁻ and epcam⁺;
(ii) MHC Class II^{lo}, CD45⁻, UEA1^{lo} and Ly51^{lo}; or
(iii) MHC Class II⁻, CD45⁻ and epcam³⁰
   is indicative of a thymic epithelial progenitor cell.

In another aspect, there is provided a method for identifying a mammalian thymic cortical epithelial progenitor cell, said method comprising screening for MHC Class II, UEA1, Ly51, epcam and/or CD45 expression by the cells of a biological sample wherein expression of a MHC Class II^{lo}, CD45⁻, UEA1^{-/lo} and Ly51⁺ phenotypic profile by a cell is indicative of a thymic cortical epithelial progenitor cell.

In still another aspect, there is provided a method for identifying a mammalian thymic medullary epithelial progenitor cell, said method comprising screening for MHC Class II, UEA1, Ly51, epcam and/or CD45 expression by the cells of a biological sample wherein expression of a MHC Class II^{lo}, CD45⁻, UEA1⁺ and Ly51⁻ phenotypic profile by a cell is indicative of a thymic medullary epithelial progenitor cell.

In one embodiment, said mammal is a human.

In another embodiment, said biological sample has been isolated from said mammal.

Still another aspect of the present invention provides a kit for identifying a thymic epithelial progenitor cell of the present invention, said kit comprising an agent for detecting MHC Class II, UEA1, Ly51, epcam and/or CD45 cell surface expression or MHC Class II, UEA1, Ly51, epcam and/or CD45 mRNA expression and, optionally, reagents useful for facilitating the detection by said agent.

The kit may also be adapted to receive a biological sample. The agent may be an antibody or other suitable detection molecule.

Preferably, the subject kit is used in the detection method of the present invention. Optionally, the kit is packaged with instructions for use eg. in a method of the present invention.

As detailed hereinbefore, the method of the present invention is unique in that it provides the first reliable means of routinely detecting specific thymic epithelial progenitor cell subpopulations. Accordingly, the applications for this technology are extensive and include, but are not limited to:

(i) Qualitatively and/or quantitatively analysing a biological sample in vitro or a mammal in vivo in order to assess the thymic epithelial progenitor cell population

This can be useful in the context of diagnosing conditions which may be characterised by aberrant thymic epithelial functionality, monitoring a tissue, or in vitro cell culture, in terms of the ongoing status of these cellular populations (for example, in order to screen for thymic epithelial viability and functionality during or following a chemotherapy or radiation treatment regime or in the context of age related thymic damage or congenital or disease induced thymic epithelial abnormality). The notion of monitoring has application in the context of, inter alia, assessing the effectiveness of a therapeutic or prophylactic treatment regimes (such as a thymic epithelial progenitor cell therapy or drug based regimes) or the effectiveness of both existing and new thymic epithelial progenitor cell lineage differentiation protocols;
(ii) Facilitating the identification and isolation of thymic epithelial progenitor cells

To date, an efficient and reliable means of enriching and purifying these cells has not been available due to both their existence and phenotypic profile having been unknown prior to the advent of this invention. Accordingly, identification of these cells and therefore the development of the current method will facilitate ongoing and more accurate characterisation of these cells. Still further, means to isolate these cells provides opportunity for the establishment of cell lines, the ongoing study of means for inducing directed differentiation, a source of cells for therapeutic, prophylactic or other clinical application (including in vivo stem cell therapies, in vivo therapies using thymic epithelial cell lineages which have been differentiated from the progenitor cell population of the invention and transplantation of tissues engineered utilising the cells isolated according to this aspect of the present invention).

Accordingly, yet another related aspect of the present invention provides a method for isolating a mammalian thymic epithelial progenitor cell, said method comprising the steps of:
(i) screening for MHC Class II, CD45, epcam, UEA1 and/or Ly51 expression by cells of a mammalian biological sample, wherein expressing a phenotypic profile selected from:
   (i) MHC Class II^{lo}, CD45⁻ and epcam⁺;
   (ii) MHC Class II^{lo}, CD45⁻, UEA1^{lo} and Ly51^{lo}; or
   (iii) MHC Class II⁻, CD45⁻ and epcam⁺
   by a cell is indicative of a thymic epithelial progenitor cell; and
(ii) isolating said cell.

Still another related aspect of the present invention provides a method for isolating a mammalian thymic cortical epithelial progenitor cell, said method comprising the steps of:
(i) screening for MHC Class II, CD45, UEA1 and/or Ly51 expression by cells of a mammalian biological sample, wherein expressing a phenotypic profile of MHC Class II^{lo}, CD45⁻, UEA1^{-/lo} and Ly51⁺ by a cell is indicative of a thymic cortical epithelial progenitor cell; and
(ii) isolating said cell.

Yet still another related aspect of the present invention provides a method for isolating a mammalian thymic medullary epithelial progenitor cell, said method comprising the steps of:
(i) screening for MHC Class II, CD45, UEA1 and/or Ly51 expression by cells of a mammalian biological sample, wherein expressing a phenotypic profile of MHC Class II^{lo}, CD45⁻, UEA1⁺ and Ly51⁻ by a cell is indicative of a thymic medullary epithelial progenitor cell; and
(ii) isolating said cell.

In one embodiment, said mammal is a human.

In another embodiment, the method for isolating a mammalian thymic epithelial progenitor cell as described herein additionally comprises isolating the mammalian biological sample.

In still another embodiment, said biological sample has been previously isolated from said mammal.

In terms of isolating these cell populations in accordance with the method of the invention, various well known techniques can be performed. Antibodies and other MHC Class II, UEA1, Ly51, epcam and/or CD45 specific cell surface binding molecules are particularly useful. For example, antibodies may be attached to a solid support to allow for separation. Procedures for separation may include magnetic separation, using antibody magnetic beads, affinity chromatography, “panning” with antibody attached to a solid matrix or any other convenient technique such as Laser Capture Microdissection. Other techniques providing particularly accurate separation include fluorescence activated cell sorting, such as exemplified herein.

These techniques can be performed as either a single-step or multi-step protocol in order to achieve the desired level of purification or enrichment.

Antibodies specific to MHC Class II, UEA1, Ly51, epcam and/or CD45 are widely known and could be identified by the person of skill in the art. For example, monoclonal antibodies that are specific to MHC Class II are commercially available from Sapphire Biosciences Pty Ltd, Australia; monoclonal antibodies that are specific to Ly51 are commercially available from BD Pharmingen, USA; monoclonal antibodies that are specific to epcam are commercially available from Sapphire Biosciences Pty Ltd, Australia; and monoclonal antibodies that are specific to CD45 are commercially available from Sapphire Biosciences Pty Ltd, Australia. UEA1 lectin is available from Vector Laboratories, Inc, USA.
(iii) Facilitating the generation of thymic epithelial progenitor cell lines and, further, the in vitro or in vivo based proliferation and/or differentiation of the progenitor cells of the present invention in the context of single cell suspensions, tissue aggregates, organoids or de novo thymus formation.

In a related aspect, there is therefore provided a method for facilitating the proliferation and/or differentiation of a thymic epithelial progenitor cell of the present invention and/or a thymic epithelial progenitor cell which cell has been identified and/or isolated in accordance with the present invention, said method comprising contacting said progenitor cell, either in vitro or in vivo, with an effective amount of a stimulus for a time and under conditions sufficient to direct the proliferation or differentiation of said cell.

In one embodiment, said progenitor cell is differentiated to a more mature thymic epithelial phenotype.

In another embodiment, said phenotype is a thymic cortical or medullary epithelial phenotype.

In yet another embodiment, there is generated a cell line.

In still another embodiment, there is generated a thymic cellular aggregate, preferably an organoid. By reference to “thymic cellular aggregate” is meant a three dimensional aggregation of cells which comprise one or more cells characteristic of the thymus. For example, such an aggregate may comprise both medullary and cortical epithelial cells or it may merely comprise one type of cell. The cells may be randomly positioned within the aggregate or they may be arranged into discrete regions, such as would be characteristic of the cortical and medullary regions of a thymic lobule. The latter may be characteristic of an organoid. In another example, the cellular aggregate may also take the form of isolated tissue.

Accordingly, in still another aspect there is provided a thymic epithelial cell line, which cell line has been generated from a thymic epithelial progenitor cell of the present invention and/or a thymic epithelial progenitor cell which has been identified and/or isolated in accordance with the method of the present invention.

Yet another aspect provides a cellular population comprising a thymic epithelial progenitor cell of the present invention and/or a thymic epithelial progenitor cell which has been identified and/or isolated in accordance with the methods of the present invention, or cells differentiated therefrom.

Still another aspect provides a composition comprising a thymic epithelial progenitor cell of the present invention and/or a thymic epithelial progenitor cell which has been identified and/or isolated in accordance with the methods of the present invention, or cells differentiated therefrom, together with a pharmaceutically acceptable carrier or excipient.

Exemplary carriers or excipients include, for example, a compound required for cell survival and/or a compound required for or that promotes cellular proliferation and/or differentiation either in vitro or in vivo, such as a cytokine, growth hormone, cellular nutrient, extracellular matrix or the like. Said composition may also comprise a compound such as a cryopreservative or antibiotic.

Reference to a “cellular population” should be understood as a reference to any form of cellular population, including a cell suspension, such as a single cell suspension (these cells may be in culture or in storage (such as a frozen sample), an adherent cell culture, an aggregate of cells, tissue or an organoid. These populations may be clonal, homogeneous or heterogeneous in terms of the range of thymic epithelial progenitor cells, or cells differentiated therefrom, which they comprise. These cellular populations may additionally comprise other cell types such as a T cell progenitor population (eg. thymocytes or stem cells, such as haematopoietic cells) or other unrelated cell types.

Accordingly, in still another aspect there is provided a tissue aggregate, which tissue aggregate comprises a thymic epithelial progenitor cell of the present invention and/or a thymic epithelial progenitor cell which has been identified and/or isolated in accordance with the methods of the present invention, or cells differentiated therefrom.

In yet still another aspect there is provided isolated thymic tissue, which thymic tissue comprises a thymic epithelial progenitor cell of the present invention and/or a thymic epithelial progenitor cell which has been identified and/or isolated in accordance with the methods of the present invention, or cells differentiated therefrom.

In still yet another aspect there is provided an organoid, which organoid comprises a thymic epithelial progenitor cell of the present invention and/or a thymic epithelial progenitor cell which has been identified and/or isolated in accordance with the methods of the present invention of cells differentiated therefrom.

By generating a 3-dimensional organoid which may be comprised of both medullary and cortical epithelium, or, alternatively, just one or other of these populations, there is provided the means for educating T cells in vitro. For example, haemopoietic cells from a patient about to undergo chemotherapy may be isolated and cultured with the organoid in order to generate a new T cell repertoire while the chemotherapy process is proceeding. This provides a T cell population which is ready for administration to the patient immediately that the chemotherapy is completed, rather than at that stage having to regenerate the patient's thymus in order to repopulate the T cell repertoire. In a post-chemotherapy immunocompromised individual, this can be crucial.

Accordingly, in another aspect there is therefore provided a method of generating a T cell, said method comprising co-culturing a T cell precursor with a thymic epithelial progenitor cell as hereinbefore defined and/or a cell differentiated therefrom for a time and under conditions sufficient to induce T cell precursor maturation.

In one embodiment, said thymic epithelial progenitor cell or cell differentiated therefrom is part of an organoid, a cellular aggregate, a tissue or an organoid.

Still more preferably, said co-culture is established with cortical and/or medullary thymic epithelial cells which have been differentiated from the progenitor cells of the present invention.

Reference to “T cell precursor” should be understood as a reference to cells which are not terminally differentiated T cells but which do exhibit the potential to differentiate along the T cell lineage. These may be stem cells or very early progenitor cells, such as haematopoietic stem cells, or they may be more mature, such as thymocytes. The “T cell” which is generated by this method may be terminally differentiated or it may be less fully differentiated, depending on the requirements of the given situation. Accordingly, the method of this aspect of the present invention may be utilised to induce any one or more of:
(i) maintaining T cell precursor viability;
(ii) inducing T cell precursor proliferation;
(iii) inducing T cell precursor differentiation; or
(iv) effecting thymocyte selection, ie. one or both of positive selection or negative selection
   those features being understood to fall within the scope of the meaning of “T cell maturation”.

Signals suitable for use in achieving proliferation or directed differentiation include IL-7, IGF-1, keratinocyte growth factor (useful for in vivo techniques since this molecule activates the thymus), growth hormone, ghrelin, LHRH. One may also use tissue culture medium conditioned by bone marrow stroma or mesenchymal cells, which are known to provide appropriate growth factors. Co-culturing with bone marrow stroma or mesenchymal cells may also be considered.

As detailed hereinbefore, this aspect of the method of the present invention can be performed either in vitro or in vivo.

Accordingly, there is also provided a method of generating a T cell in a mammal, said method comprising administering to said mammal a thymic epithelial progenitor cell of the present invention and/or a thymic epithelial progenitor cell which has been identified and/or isolated in accordance with the methods of the present invention and/or a cell differentiated therefrom, under conditions sufficient to induce T cell precursor maturation.

It should be understood that the T cell precursors which are the subject of maturation may be endogenous to the mammal or they may also have been administered from an ex vivo source.

The present invention also extends to the isolated T cells generated in accordance with these aspects of the invention, a composition comprising these cells together with a pharmaceutical excipient and their use in the treatment of a mammal.

In accordance with all the methods herein described, it should be understood that said thymic epithelial progenitor cells or cells differentiated therefrom may be used or administered in any suitable form, including as a cell suspension, pharmaceutical composition as hereinbefore described or as a cellular aggregate, such as a tissue or organoid. Accordingly, all references to “thymic epithelial progenitor cells” or “cells differentiated therefrom” includes reference to all forms of these cells and cellular populations. Means of achieving this are described in more detail in the next section. In terms of in vitro technology, there is now provided means of routinely and reliably producing differentiated thymic epithelial cells on either a small scale or on a larger scale. In terms of small scale production, which may be effected in tissue culture flasks for example, this may be particularly suitable for producing populations of cells for a given individual and in the context of a specific condition. One means of achieving large scale production in accordance with the method of the invention is via the use of a bioreactor.

Bioreactors are designed to provide a culture process that can deliver medium and oxygenation at controlled concentrations and rates that mimic nutrient concentrations and rates in vivo. Bioreactors have been available commercially for many years and employ a variety of types of culture technologies. Of the different bioreactors used for mammalian cell culture, most have been designed to allow for the production of high density cultures of a single cell type and as such find use in the present invention. Typical application of these high density systems is to produce as the end-product, a conditioned medium produced by the cells. This is the case, for example, with hybridoma production of monoclonal antibodies and with packaging cell lines for viral vector production. However, these applications differ from applications where the therapeutic end-product is the harvested cells themselves, as may occur in the present invention.

Once operational, bioreactors provide automatically regulated medium flow, oxygen delivery, and temperature and pH controls, and they generally allow for production of large numbers of cells. Bioreactors thus provide economies of labour and minimization of the potential for mid-process contamination, and the most sophisticated bioreactors allow for set-up, growth, selection and harvest procedures that involve minimal manual labour requirements and open processing steps. Such bioreactors optimally are designed for use with a homogeneous cell mixture or aggregated cell populations as contemplated by the present invention. Suitable bioreactors for use in the present invention include but are not limited to those described in U.S. Pat. No. 5,763,194, U.S. Pat. Nos. 5,985,653 and 6,238,908, U.S. Pat. No. 5,512,480, U.S. Pat. Nos. 5,459,069, 5,763,266, 5,888,807 and 5,688,687.

With any large volume cell culture, several fundamental parameters require almost constant control. Cultures must be provided with the medium that allows for, in the present invention, thymic epithelial progenitor cell maintenance, thymic epithelial progenitor cell proliferation and differentiation (perhaps in the context of several separate differentiation cultures and conditions) as well as final cell culture/preservation. Typically, the various media are delivered to the cells by a pumping mechanism in the bioreactor, feeding and exchanging the medium on a regular basis. The exchange process allows for by-products to be removed from the culture. Growing cells or tissue also requires a source of oxygen. Different cell types can have different oxygen requirements. Accordingly, a flexible and adjustable means for providing oxygen to the cells is a desired component.

Depending on the particular culture, even distribution of the cell population and medium supply in the culture chamber can be an important process control. Such control is often achieved by use of a suspension culture design, which can be effective where cell-to-cell interactions are not important. Examples of suspension culture systems include various tank reactor designs and gas-permeable plastic bags. For cells that do not require assembly into a three-dimensional structure or require proximity to a stromal or feeder layer such suspension designs may be used. Also contemplated are 3 dimensional cultures which utilise a range of biological and synthetic scaffolds (for example for engineering specific thymic structures). Accordingly, ex vivo de novo thymus formation is contemplated by the present invention.

Efficient collection of the cells at the completion of the culture process is an important feature of an effective cell culture system. One approach for production of cells as a product is to culture the cells in a defined space, without physical barriers to recovery, such that simple elution of the cell product results in a manageable, concentrated volume of cells amenable to final washing in a commercial, closed system cell washer designed for the purpose. Optimally, the system would allow for addition of a pharmaceutically acceptable carrier, with or without preservative, or a cell storage compound, as well as providing efficient harvesting into appropriate sterile packaging. Optimally, the harvest and packaging process may be completed without breaking the sterile barrier of the fluid path of the culture chamber.

With any cell culture procedure, a major concern is sterility. When the product cells are to be transplanted into patients (often at a time when the patient is ill or immunocompromised), absence of microorganisms is mandated. An advantage of the present cell production device over manual processes is that, as with many described bioreactor systems, once the culture is initiated, the culture chamber and the fluid pathway is maintained in a sterile, closed environment.
(iv) Therapeutic or prophylactic treatment of patients—thymic regeneration

The development of the present invention has also now facilitated the development of means for therapeutically or prophylactically treating subjects exhibiting aberrant thymic epithelial cellular functioning, based on administering to these patients thymic epithelial progenitor cells or partially or fully differentiated cells of thymic epithelial progenitor origin, which cells may have been identified and isolated according to the screening method aspect of the invention, to regenerate the thymus. In fact, the epithelial cells of the present invention are activated, upon induction of thymic damage, for the purpose of regeneration. This method can be applied to a wide range of conditions including, but not limited to, thymic damage caused by a chemotherapy, radiation or immunosuppressive drug treatment regime, age related thymic deterioration, LHRH unresponsiveness, congenital thymic abnormality or disease induced thymic deterioration.

Reference to a condition characterised by “aberrant or otherwise unwanted thymic epithelial functioning” should be understood as a reference to any condition which is due, at least in part, to a defective thymic epithelial cell population, in particular thymic epithelial cell degeneration. This may correspond to either a homogeneous or heterogeneous population of thymic epithelial cells. The subject defect should be understood as a reference to any structural or functional feature of the thymic epithelial cell which is not normal. Without limiting the present invention to any one theory or mode of action, the degeneration of thymic epithelium results in a loss of thymic activity, specifically the reduction or cessation of thymocyte differentiation and release to the periphery. Although this is, in fact, a normal physiological process which is evident as progressive thymic involution commencing at puberty, this process is nevertheless not necessarily desirable, particularly in the context of adults who become immunocompromised due to disease or lymphocyte ablative therapies such as are a necessary side effect of most chemotherapy and radiation treatment regimes.

Accordingly, another aspect of the present invention is directed to a method of therapeutically and/or prophylactically treating a condition in a mammal, which condition is characterised by aberrant or otherwise unwanted thymic epithelial cell structure or functioning, said method comprising administering to said mammal an effective number of the thymic epithelial progenitor cells of the present invention and/or thymic epithelial progenitor cells which have been identified and/or isolated in accordance with the methods of the present invention, or cells differentiated therefrom for a time and under conditions sufficient to induce proliferation or differentiation or to otherwise induce thymic formation.

The present invention therefore provides a method for producing or regenerating a thymic tissue in a subject.

Accordingly, a related aspect of the present invention is directed to a method for producing or regenerating thymus tissue in a mammal, said method comprising administering to said mammal an effective number of the thymic epithelial progenitor cells of the present invention and/or thymic epithelial progenitor cells which have been identified and/or isolated in accordance with the methods of the present invention, or cells differentiated therefrom for a time and under conditions sufficient to proliferate, differentiate and/or otherwise form thymic tissue.

In one embodiment, the methods of this aspect of the present invention are directed to regenerating both the cortex and medulla. In another embodiment, this aspect of the present invention is directed to regenerating only one of medulla or cortex.

Reference to “administering” to an individual an effective number of thymic epithelial cells should be understood to include reference to either introducing into the mammal an ex vivo population of thymic epithelial progenitor cells or cells differentiated therefrom or introducing into the mammal an effective amount of a stimulus which will act on thymic epithelial progenitor cells located in vivo, and preferably located in situ, to generate a differentiated thymic epithelial cell. With respect to this latter embodiment, the cell may be one which has always been present in the individual (that is, it has never been removed from the individual, such as an adult thymic epithelial progenitor cell) or it may be one which was previously located ex vivo and has been introduced into the individual whereby its in vivo differentiation will subsequently be effected. This may occur where a thymic epithelial progenitor cell line is created using nuclear material derived from the patient in issue. In this regard, it may be desirable to manipulate, culture, mark or otherwise treat the cell ex vivo in order to prepare it for in vivo differentiation but to conduct the actual step of either thymic epithelial progenitor cell differentiation or terminal differentiation in the in vivo, and even more preferably in situ, environment.

Preferably, said method is performed by administering an ex vivo population of thymic epithelial progenitor cells or cells differentiated in vitro therefrom.

In accordance with this preferred embodiment, the subject cells are preferably autologous cells which are identified, isolated and/or differentiated ex vivo and transplanted back into the individual from which they were originally harvested. However, it should be understood that the present invention nevertheless extends to the use of cells derived from any other suitable source where the subject cells exhibit the same major histocompatability profile as the individual who is the subject of treatment. Accordingly, such cells are effectively autologous in that they would not result in the histocompatability problems which are normally associated with the transplanting of cells exhibiting a foreign MHC profile. Such cells should be understood as falling within the definition of “autologous”. For example, under certain circumstances it may be desirable, necessary or of practical significance that the subject cells are isolated from a genetically identical twin, or from an embryo generated using gametes derived from the subject individual or cloned from the subject individual. The cells may also have been engineered to exhibit the desired major histocompatability profile or at least an MHC profile which would exhibit reduced reactivity due to the compatibility of at least some of the major MHC loci. The use of such cells overcomes or at least reduces the difficulties which are inherently encountered in the context of tissue and organ transplants. However, where it is not possible or feasible to isolate or generate autologous thymic epithelial cells, it may be necessary to utilise allogeneic stem cells. “Allogeneic” thymic epithelial cells are those which are isolated from the same species as the subject being treated but which exhibit a different MHC profile. Although the use of such cells in the context of therapeutics would likely necessitate the use of immunosuppression treatment, this problem can nevertheless be minimised by use of cells which exhibit an MHC profile exhibiting similarity to that of the subject being treated, such as thymic epithelial population which has been isolated/generated from a relative such as a sibling, parent or child or one which has been genetically engineered to exhibit improved matching at some of the major MHC loci, as detailed above. The present invention should also be understood to extend to xenogeneic transplantation. That is, the thymic epithelial cells which are differentiated in accordance with the method of the invention and introduced into a patient, are isolated from a species other than the species of the subject being treated. It should be understood that these principles also apply to the situation where a population of thymic epithelial cells is administered to a patient for the purpose of effecting differentiation in vivo.

Without limiting the present invention to any one theory or mode of action, even partial restoration of thymic tissue structure or functioning will act to ameliorate the symptoms of many conditions. Accordingly, reference to an “effective number” means that number of cells necessary to at least partly attain the desired effect, or to delay the onset of, inhibit the progression of, or halt altogether the onset or progression of the particular condition being treated. Such amounts will depend, of course, on the particular conditions being treated, the severity of the condition and individual patient parameters including age, physical conditions, size, weight, physiological status, concurrent treatment, medical history and parameters related to the disorder in issue. One skilled in the art would be able to determine the number of cells and tissues of the present invention that would constitute an effective dose, and the optimal mode of administration thereof without undue experimentation, this latter issue being further discussed hereinafter. These factors are well known to those of ordinary skill in the art and can be addressed with no more than routine experimentation. It is preferred generally that a maximal cell number be used, that is, the highest safe number according to sound medical judgement. It will be understood by those of ordinary skill in the art, however, that a lower cell number may be administered for medical reasons, psychological reasons or for any other reasons.

As hereinbefore discussed, it should also be understood that although the method of the present invention is predicated on the introduction of differentiated cells to an individual suffering a condition as herein defined, it is not necessarily the case that every cell of the population introduced to the individual will exhibit the desired phenotype. For example, where the thymic epithelial progenitor population of the present invention has undergone differentiation and is administered in total, there may exist a proportion of cells which have not undergone differentiation to a cell exhibiting the requisite cortical or medullary phenotype. The present invention is therefore achieved provided the relevant portion of the cells thereby introduced constitute the “effective number” as defined above. However, in a particularly preferred embodiment the population of cells which have undergone differentiation will be subjected to the identification of successfully differentiated cells, their isolation and introduction to the subject individual. This provides a means for selecting either a heterogeneous population of thymic epithelial cells, such as may occur where tissue is induced to develop, or to select out a specific subpopulation of cells for administration. The type of method which is selected for application will depend on the nature of the condition being treated. However, it is expected that in general it will be desirable to administer a pure population of differentiated cells in order to avoid potential side effects such as teratoma formation by remnant stem cells. Alternatively, in some instances it may be feasible to subject a population of thymic epithelial progenitor cells to differentiation and provided that this population, as a whole, are shown to exhibit the requisite functional activity, this population as a whole may be introduced into the subject individual without the prior removal of non-thymic epithelial cells. Accordingly, reference to “an effective number”, in this case, should be understood as a reference to the total number of cells required to be introduced such that the number of differentiated cells is sufficient to produce the level of activity which achieves the object of the invention, being the treatment of the subject condition.

As detailed hereinbefore, differentiation of the subject cells can be performed in vivo or in vitro. In the latter situation, the subject cell will then require introduction into the subject individual. Where the cells are differentiated in vitro, the subject cells are preferably ones which were isolated from the individual to be treated (i.e. autologous cells). However, the present invention nevertheless extends to the use of cells sourced elsewhere, such as syngeneic cells from an identical twin or cells from an embryo which exhibits the same major histocompatability profile as that of the individual in question. To the extent that the cells are differentiated in vitro, the cells may be subsequently introduced into an individual by any suitable method. For example, cell suspensions may be introduced by direct injection or inside a blood clot whereby the cells are immobilised in the clot thereby facilitating transplantation. The cells may also be encapsulated prior to transplantation. Encapsulation is a technique which is useful for preventing the dissemination of cells which may continue to proliferate (i.e. exhibit characteristics of immortality) or for minimising tissue incompatibility rejection issues. However, the usefulness of encapsulation will depend on the function which the transplanted cells are required to provide. For example, if the transplanted cells are required primarily for the purpose of secreting a soluble factor, a population of encapsulated cells will likely achieve this objective. However, if the transplanted cells are required for their structural properties, which is more likely to be the case in terms of reactivating the thymocyte education functioning of the thymus, the cells will likely be required to integrate with the existing tissue scaffold. Encapsulated cells would not be able to do this efficiently.

The cells which are administered to the patient can be administered as single or multiple doses by any suitable route. Preferably, and where possible, a single administration is utilised. Administration via injection can be directed to various regions of a tissue or organ, depending on the type of repair required.

It would be appreciated that in accordance with these aspects of the present invention, the cells which are administered to the patient may take any suitable form, such as being in a cell suspension or taking the form of a tissue or organoid graft. In terms of generating a single cell suspension, the differentiation protocol may be designed such that it favours the maintenance of a cell suspension. Alternatively, if cell aggregates or tissues form, these may be dispersed into a cell suspension. In terms of utilising a cell suspension, it may also be desirable to select out specific subpopulations of cells for administration to a patient, such as terminally differentiated cells. To the extent that it is desired that a tissue is transplanted into a patient, this will usually require surgical implantation (as opposed to administration via a needle or catheter). Alternatively, a portion, only, of this tissue could be transplanted. In another example, engineered tissues can be generated via standard tissue engineering techniques, for example by seeding a tissue engineering scaffold having the designed form with the cells and tissues of the present invention and culturing the seeded scaffold under conditions enabling colonization of the scaffold by the seeded cells and tissues, thereby enabling the generation of the formed tissue. The formed tissue is then administered to the recipient, for example using standard surgical implantation techniques. Suitable scaffolds may be generated, for example, using biocompatible, biodegradable polymer fibers or foams, comprising extracellular matrix components, such as laminins, collagen, fibronectin, etc. Detailed guidelines for generating or obtaining suitable scaffolds, culturing such scaffolds and therapeutically implanting such scaffolds are available in the literature (for example, refer to Kim S. S. and Vacanti J. P., 1999. Semin Pediatr Sung. 8:119, U.S. Pat. No. 6,387,369 to Osiris, Therapeutics, Inc.; U.S. Pat. App. No. US20020094573A1 to Bell E.).

In accordance with the method of the present invention, other proteinaceous or non-proteinaceous molecules may be co-administered either with the introduction of the subject cells or prior or subsequently thereto. By “co-administered” is meant simultaneous administration in the same formulation or in different formulations via the same or different routes or sequential administration via the same or different routes. By “sequential” administration is meant a time difference of from seconds, minutes, hours or days between the introduction of these cells and the administration of the proteinaceous or non-proteinaceous molecules or the onset of the functional activity of these cells and the administration of the proteinaceous or non-proteinaceous molecule. For example, depending on the nature of the condition being treated, it may be necessary to maintain the patient on a course of medication to alleviate the symptoms of the condition until such time as the transplanted cells become integrated and fully functional. Alternatively, at the time that the condition is treated, it may be necessary to commence the long term use of medication to prevent re-occurrence of the damage.

It should also be understood that the method of the present invention can either be performed in isolation to treat the condition in issue or it can be performed together with one or more additional techniques designed to facilitate or augment the subject treatment. These additional techniques may take the form of the co-administration of other proteinaceous or non-proteinaceous molecules, as detailed hereinbefore.

In a related aspect of the present invention, the subject undergoing treatment or prophylaxis may be any human or animal in need of therapeutic or prophylactic treatment. In this regard, reference herein to “treatment” and “prophylaxis” is to be considered in its broadest context. The term “treatment” does not necessarily imply that a mammal is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity of the onset of a particular condition. “Treatment” may also reduce the severity of an existing condition.
(v) Therapeutic or prophylactic treatment of patents—tolerance induction

The induction of tolerance to self antigens is one of the major educative functions of the thymic medullary epithelium. To this end, the onset of autoimmunity is a classical example of the breakdown of self-tolerance to peripheral antigens. Transplantation rejection is somewhat of an immunological anomaly due to the fact that self T cells are recognising and responding to non-self MHC:peptide, this not being entirely consistent with the theory of self-MHC restriction. Nevertheless, the response to foreign tissue is often rapid and severe where extensive mismatch of donor tissue across an individual's MHC haplotype exists. To this end, overcoming transplant rejection has, to date, focussed on reducing MHC mismatch by screening for donors who are more closely matched. However, due to the extensive diversity of MHC haplotypes across the population, it is often difficult to find a suitable donor. The method of the present invention, however, provides a means for effectively generating a newly educated patient T cell population which is tolerant to some or all of the MHC loci comprising a donor tissue haplotype. Accordingly, even if tissue rejection cannot be entirely prevented, its severity may be reduced sufficiently to effect better manageability of tissue rejection problems.

Given that a major mechanism underlying the prevention of T cells reacting against self antigens is due to the negative selection process by thymic medullary epithelial cells, the ability to generate new thymic tissue which comprises thymic medullary epithelial cells from a potential organ or tissue donor or which are at least matched more closely at the MHC loci, has major importance in terms of the prevention of graft rejection. By ablating peripheral T cells and educating new thymocytes in a chimeric thymus which express both self and non-self MHC, there is provided a means of generating a tolerant T cell population in which reactive thymocytes have been selected against.

The thymic epithelial cells which are used in the context of this aspect of the present invention are either progenitors of donor origin or progenitors which have been engineered to express a matching or at least more compatible MHC profile. These are administered to the patient as hereinbefore defined in order to facilitate their intrathymic differentiation to cortical and/or medullary epithelium. Alternatively, fully differentiated cells may be administered such as to effect their localisation and integration into the thymus.

Preferably, the donor cells are administered to the recipient and migrate through the peripheral blood system to the thymic tissue. These cells become integrated into the new thymic tissue and produce functional epithelial cells. Depending on the state of the patient, it may also be necessary to administer a haemopoietic stem cell population to facilitate the generation of a source of thymocytes. The result is a chimeric organ which generates T cells that are tolerant to both the host and donor MHC and circulate in the peripheral blood of the host.

In addition to the notion of inducing tolerance to non-self MHC, the identification of the cells of the present invention has also enabled the development of methods to induce tolerance to specific antigens such as autoantigens (these being self antigens to which tolerance has either been broken down or was not initially properly generated) and innocuous antigens (eg. hypersensitivity inducing antigens). By transfecting the subject thymic epithelial progenitor cells to express the antigens of interest and colonising or otherwise integrating these cells into the thymus, there is provided a means of facilitating the presentation of these antigens to the developing thymocytes such that tolerance induction is effected. By preceding this step with a step of ablating peripheral T cells (techniques for which are in common use and known to the person of skill in the art), pre-existing autoreactive T cells can be eliminated thereby facilitating reconstitution of the immune system with tolerant cells.

Accordingly, the present invention provides a method for inducing tolerance to an antigen in a mammal, said method comprising reducing, ablating or otherwise downregulating the functionality of peripheral T cells in said mammal and administering to said mammal an effective number of thymic epithelial progenitor cells of the present invention and/or thymic epithelial progenitor cells which have been identified and/or isolated in accordance with the method of the present invention, or cells differentiated therefrom, which cells express at least one epitope of the subject antigen.

As detailed hereinbefore, the subject antigen may be naturally expressed (such as the MHC of an allogeneic population of thymic epithelial progenitor cells) or it may be the result of genetic manipulation. It should be understood that the subject thymic epithelial progenitor cells may express one or more epitopes or antigens of interest, such as where more than one individual MHC molecule from a haplotype of interest is sought to be expressed.

In still another aspect, there is provided a method of therapeutically or prophylactically inducing graft tolerance in a mammal, said method comprising reducing, ablating or otherwise downregulating the functionality of peripheral T cells in said mammal and administering to said mammal an effective number of thymic epithelial progenitor cells and/or thymic epithelial progenitor cells which have been identified and/or isolated in accordance with the method of the present invention, or cells differentiated therefrom, which cells express at least one MHC molecule of said graft.

Preferably, said thymic epithelial progenitor cells or cells differentiated therefrom are of donor tissue origin.

In another embodiment, said graft is an allograft or xenograft.

In still another embodiment, said thymic progenitor epithelial cells or cells differentiated therefrom are cells which have been genetically modified to express a partial or complete match with the MHC haplotype of the prospective donor.

In still another aspect there is provided a method of therapeutically or prophylactically treating graft rejection, said method comprising reducing, ablating or otherwise downregulating the functionality of peripheral T cells in said mammal and administering to said mammal an effective number of thymic epithelial progenitor cells and/or thymic epithelial progenitor cells which have been identified and/or isolated in accordance with the method of the present invention, or cells differentiated therefrom, which cells express at least one MHC molecule of said graft.

In yet still another embodiment, said antigen is an allergen or an autoimmune antigen.

Accordingly, in another aspect, there is provided a method of therapeutically or prophylactically treating an autoimmune condition in a mammal, said method comprising reducing, ablating or otherwise downregulating the functionality of autoreactive peripheral T cells in said mammal and administering to said mammal an effective number of thymic epithelial progenitor cells of the present invention and/or thymic epithelial progenitor cells which have been identified and/or isolated in accordance with the method of the present invention, or cells differentiated therefrom, which cells express at least one epitope of the autoantigen to which said autoimmune condition is directed.

In one embodiment, said autoimmune condition is acute disseminated encephalomyelitis, Addison's disease, ankylosing spondylitis, antiphospholipid antibody syndrome, diabetes mellitus type 1, Guillain-Barre syndrome, Hashimoto's disease, idiopathic thrombocytopenic purpura, Goodpasture's syndrome, Graves' disease, lupus erythematosus, multiple sclerosis, myasthenia gravis, rheumatoid arthritis, pemphigus, Sjogren's syndrome, temporal arthritis, aplastic anaemia, autoimmune hepatitis, autoimmune oophoritis, celiac disease, Crohn's disease, gestational pemphigoid, Kawasaki's disease, mixed connective tissue disease, opsoclonus myoclonus syndrome, Ord's thyroiditis, pernicious anaemia, polyarthritis in dogs, primary biliary cirrhosis, Reiter's syndrome, Takayasu's arteritis, vitiligo, warm autoimmune haemolytic anaemia, Wegener's granulomatosis.

In yet another aspect, there is provided a method of therapeutically or prophylactically treating a hypersensitivity condition in a mammal, said method comprising reducing, ablating or otherwise downregulating the functionality of antigen reactive peripheral T cells in said mammal and administering to said mammal an effective number of thymic epithelial progenitor cells of the present invention and/or thymic epithelial progenitor cells which have been identified and/or isolated in accordance with the method of the present invention, or cells differentiated therefrom, which cells express at least one epitope of the antigen to which said hypersensitivity condition is directed.

In one embodiment, said allergen is a pollen allergen, dust mite allergen, gross allergen, food allergen, such as a nut allergen, bee venom allergen or a latex allergen.
(vi) Therapeutic or Prophylactic Treatment of Patients—Protein Expression

In yet another aspect, the present invention envisages the administration of thymic epithelial progenitor cells, or cells which have been differentiated therefrom, which have been engineered to express a protein or gene of interest, such as a cytokine, growth factor, hormone (eg. growth hormone), insulin-like growth factor (IGF1), KGF enzyme, antiviral gene or drug resistance gene. By engineering the subject cells to express these proteins in an inducible system, there is provided means for effecting the control of expression of the subject protein. This is of particular value where the protein is a growth factor or other cytokine which is designed to induce functional effects in the thymus at specific time points. This may be useful, for example, after chemotherapy or irradiation induced damage or in the context of autoimmunity, HIV, immunosuppression or antiretroviral therapy.

There is therefore provided a method of treating a mammal, said method comprising administering to said mammal a population of thymic epithelial progenitor cells of the present invention and/or thymic epithelial progenitor cells which have been identified and/or isolated in accordance with the method of the present invention, or cells differentiated therefrom, which cells express a protein, anti-retroviral protein or gene of interest.

In one embodiment said protein or gene is an autoantigen, allergen, cytokine, growth factor, hormone, enzyme or other soluble factor, an anti-viral protein or a drug resistance gene.

With respect to the general notion of genetically modifying the cells of the present invention, the genes or gene fragments are used in a stably expressible form. The term “stably expressible form” as used herein means that the product (RNA and/or protein) of the gene or gene fragment (“functional fragment”) is capable of being expressed on at least a transient basis in a host cell after transfer of the gene or gene fragment to that cell, as well as in that cell's progeny after division and/or differentiation. This requires that the gene or gene fragment, whether or not contained in a vector, has appropriate signalling sequences for transcription of the DNA to RNA. Additionally, when a protein coded for by the gene or gene fragment is the active molecule that affects the patient's condition, the DNA will also code for translation signals.

In most cases the genes or gene fragments will be contained in vectors. Those of ordinary skill in the art are aware of expression vectors that may be used to express the desired RNA or protein.

Expression vectors are vectors that are capable of directing transcription of DNA sequences contained therein and translation of the resulting RNA. Expression vectors are capable of replication in the cells to be genetically modified, and include plasmids, bacteriophage, viruses, and minichromosomes. Alternatively the gene or gene fragment may become an integral part of the cell's chromosomal DNA. Recombinant vectors and methodology are in general well-known.

Expression vectors useful for expressing the proteins of the present disclosure contain an origin of replication. Suitably constructed expression vectors contain an origin of replication for autonomous replication in the cells, or are capable of integrating into the host cell chromosomes. Such vectors may also contain selective markers, a limited number of useful restriction enzyme sites, a high copy number, and strong promoters. Promoters are DNA sequences that direct RNA polymerase to bind to DNA and initiate RNA synthesis; strong promoters cause such initiation at high frequency.

In one embodiment, the DNA vector construct comprises a promoter, enhancer, and a polyadenylation signal. The promoter may be selected from the group consisting of HIV, such as the Long Terminal Repeat (LTR), Simian Virus 40 (SV40), Epstein Barr virus, cytomegalovirus (CMV), Rous sarcoma virus (RSV), Moloney virus, mouse mammary tumor virus (MMTV), human actin, human myosin, human hemoglobin, human muscle creatine, human metalothionein. In one embodiment, an inducible promoter is used so that the amount and timing of expression of the inserted gene or polynucleotide can be controlled.

The enhancer may be selected from the group including, but not limited to, human actin, human myosin, human hemoglobin, human muscle creatine and viral enhancers such as those from CMV, RSV and EBV. The promoter and enhancer may be from the same or different gene.

The polyadenylation signal may be selected from the group consisting of: LTR polyadenylation signal and SV40 polyadenylation signal, particularly the SV40 minor polyadenylation signal among others.

The expression vectors of the present disclosure are operably linked to DNA coding for an RNA or protein to be used in this invention, i.e., the vectors are capable of directing both replication of the attached DNA molecule and expression of the RNA or protein encoded by the DNA molecule. Thus, for proteins, the expression vector must have an appropriate transcription start signal upstream of the attached DNA molecule, maintaining the correct reading frame to permit expression of the DNA molecule under the control of the control sequences and production of the desired protein encoded by the DNA molecule. Expression vectors may include, but are not limited to, cloning vectors, modified cloning vectors and specifically designed plasmids or viruses. In one embodiment, an inducible promoter may be used so that the amount and timing of expression of the inserted gene or polynucleotide can be controlled.

One having ordinary skill in the art can produce DNA constructs which are functional in cells. In order to test expression, genetic constructs can be tested for expression levels in vitro using tissue culture of cells of the same type of those to be genetically modified.

Standard recombinant methods can be used to introduce genetic modifications into the cells being used for gene therapy. For example, retroviral vector transduction of cultured epithelial progenitor cells is one successful method [Belmont and Jurecic, 1997, Gene Therapy Protocols, Humana Press, pp. 223-40; Bahnson et al. 1997, Gene Therapy Protocols, Humana Press, pp. 249-263]. Additional vectors include, but are not limited to, those that are adenovirus derived or lentivirus derived, and Moloney murine leukemia virus-derived vectors.

Also useful are the following methods: particle-mediated gene transfer such as with the gene gun [Yang and Ziegelhoffer, 1997, Particle Bombardment Technology for Gene Transfer, Oxford University Press, pp. 117-41], liposome-mediated gene transfer, coprecipitation of genetically modified vectors with calcium phosphate [Graham and Van Der Eb, 1973, Virology 52:456-57, electroporation [Potter et al. 1984, Proc. Natl. Acad. Sci. USA 81:7161-65], and microinjection [Capecchi 1980, Cell 22:479-88], as well as any other method that can stably transfer a gene or oligonucleotide, which may be in a vector, into the epithelial progenitor cell such that the gene will be expressed at least part of the time.

The present invention provides methods for gene therapy. This is accomplished by the administration of genetically modified thymic epithelial progenitor cells, or cells differentiated therefrom, to a recipient. The thymic epithelial can be obtained by sorting these cells from the patient's tissue. Alternatively, they may be isolated from a different individual or from a stock source, such as a cell line, previously harvested frozen stock or the like. The number of cells can be enhanced in several ways, including (but not limited to) by administering appropriate cytokines to the patient prior to collecting cells, culturing the collected cells in appropriate conditions to effect their expression.

In one aspect, the genetically modified cells are administered to the patient and migrate through the peripheral blood system to the thymic tissue. These cells become integrated into the thymic tissue and produce epithelial tissue expressing the genetic modification of the altered cells. Alternatively, the cells are administered directly into the thymus or into the liver.

The present invention also provides the epithelial progenitor cells of the present invention, or cells differentiated therefrom, for use in medicine.

The present invention additionally provides the thymic epithelial progenitor cells of the present invention or cells differentiated therefrom for use in treating a condition in a mammal, which condition is characterised by aberrant or otherwise unwanted thymic epithelial cell structure.

There is also provided the thymic epithelial progenitor cells of the present invention or cells differentiated therefrom for use in producing or regenerating thymus tissue in a mammal.

In another aspect there is provided the thymic epithelial progenitor cells of the present invention or cells differentiated therefrom for use in inducing the tolerance to an antigen in a mammal.

In still another aspect there is provided the thymic epithelial progenitor cells of the present invention or cells differentiated therefrom for use in therapeutically or prophylactically treating graft rejection, an autoimmune condition or an allergy or hypersensitivity condition.

In yet still another aspect there is provided the thymic epithelial progenitor cells of the present invention or cells differentiated therefrom for use in treating a mammal, which cells express a protein or gene of interest.

In a further aspect there is provided the thymic epithelial progenitor cells of the present invention or cells differentiated therefrom in the membrane of a medicament for the therapeutic or prophylactic treatment of a condition in a mammal, which condition is selected from:
(i) a condition characterised by aberrant or otherwise unwanted thymic epithelial cell structure;
(ii) graft rejection;
(iii) an autoimmune condition; or
(iv) an allergy or hypersensitivity condition.

In another further aspect there is provided thymic epithelial progenitor cells of the present invention or cells differentiated therefrom in the manufacture of a medicament for producing or regenerating the thymus or inducing tolerance to an antigen.

The identification of novel thymic epithelial cell populations, the development of a method for routinely identifying these cells based on phenotypic characteristics and differentiating thymic epithelial cells therefrom in vitro has now facilitated the development of in vitro based screening systems for testing the effectiveness and toxicity of existing or potential treatment or culture regimes.

Thus, according to yet another aspect of the present invention, there is provided a method of assessing the effect of a treatment or culture regime on the phenotypic state of the thymic epithelial progenitor cell of the present invention and/or thymic epithelial progenitor cells which have been identified and/or isolated in accordance with the method of the present invention, or cells differentiated therefrom, said method comprising subjecting said cells to said treatment regime and screening for an altered functional or phenotypic state.

By “altered” is meant that one or more of the phenotypic or functional parameters which are the subject of analysis are changed relative to untreated cells. This may be a desirable outcome where the treatment regime in issue is designed to improve cellular functioning. However, where the treatment regime is associated with a detrimental outcome, this may be indicative of toxicity and therefore the unsuitability for use of the treatment regime. It is now well known that the differences which are observed in terms of the responsiveness of an individual to a particular drug are often linked to the unique genetic makeup of that individual. Accordingly, the method of the present invention provides a valuable means of testing either an existing or a new treatment regime on thymic epithelial cells which are generated utilising nuclear material derived from the individual in issue. This provides a unique means for evaluating the likely effectiveness of a drug on an individual's cellular system prior to administering the drug in vivo. Where a patient is extremely unwell, the physiological stress which can be caused by a treatment regime which causes an unwanted outcome can be avoided or at least minimised.

Accordingly, this aspect of the present invention provides a means of optimising a treatment which may be designed to target the thymus or in respect of which such targeting is a potential side effect. However the method can also be used to assess the toxicity of a treatment, in particular a treatment with a drug. For example, the screening method of this aspect of the present invention is useful in the context of assessing the effect of cytotoxic drugs which one may be seeking to use (eg. chemotherapy drugs), the drug conditions in the context of which organ transplantation would be performed or therapeutic drugs (eg. anti-retroviral drugs).

Hence the method of the present invention can be used to screen and/or test drugs, other treatment regimes or culture conditions. In the context of assessing phenotypic or functional changes, this aspect of the present invention can be utilized to monitor for changes to the gene expression profiles of the subject cells and tissues, their cell surface phenotypic profiles, morphology or functionality. Thus, the method according to this aspect of the present invention can be used to determine, for example, gene expression pattern changes in response to a treatment.

Preferably, the treatment to which the cells or tissues of the present invention are subjected is an exposure to a compound. Preferably, the compound is a drug. Alternatively the compound can be a growth factor or differentiation factor. To this end, it is highly desirable to have available a method which is capable of predicting such side effects on thymic epithelial tissue prior to administering the drug.

The present invention is further described by reference to the following non-limiting examples.

### Example 1

### Materials and Methods

### Animals

Male and female C57B16 mice aged at 4-8 weeks were obtained from Monash Animal Services and housed under SPF conditions.

### Chemotherapy

### Cyclosporine

Mice were given 15 mg/kg/day Cyclosporine (Novartis, Australia) i.p. in PBS for 14 days. Control mice were injected with the same volume of PBS.

### Cyclophosphamide

Mice were given 100 mg/kg/day of Cyclophosphamide ( ) i.p. in PBS for 2 days. Control mice were given PBS alone.

### Dexamethasone

Mice were given either 20 mg/kg Dexamethasone (Lyppards, Australia) or the equivalent volume of PBS as a single i.p. injection.

In all experiments, the final day of treatment was designated as Day 0. Mice were then humanely killed by CO2 asphyxiation at various timepoints.

### Reversal of Age-Related Thymic Atrophy

Mice (young 2-3 months, middle aged 8-12 months and old aged >18 months) were either surgically castrated or treated with the agonist hormone LHRH (lucin, TAP Pharmaceuticals, Chicago Ill.)

### Individual Thymic Stromal Cell Digestion

This procedure was adapted from a previously described protocol (Gray et al, 2002). Each thymus was thoroughly cleaned of fat and connective tissue and its capsule nicked with fine scissors. Each thymus was then incubated at 37° C. for 10 minutes in 2 mL of 0.125% (w/v) Collagenase D with 500 μL of 0.1% (w/v) DNAse I (both from Roche, Germany) in RPMI-1640, with regular agitation. The supernatant was collected, kept on ice, and the digestion repeated using the remaining settled thymic fragments. After 3 digestions, the remaining aggregates were incubated for 10 minutes with 1 mL of 0.125% (w/v) Collagenase/Dispase (Roche, Germany) with 500 μL 0.1% (w/v) DNAse I in RPMI-1640. Cells from all supernatant fractions were then pooled and centrifuged at 472 gₘₐₓ for 5 minutes, then resuspended in cold EDTA/FACS buffer (5 mM EDTA in PBS with 2% FCS and 0.02% NaN₃). Cells were filtered through 100 μm mesh. Cell counts were performed using a Z2 Coulter Counter (Beckman Coulter USA).

### Immunofluorescent Staining and Acquisition

Cells were washed in cold EDTA/FACS buffer and 5×10⁶ cells dispensed into 96 well round-bottom plates, Cells were suspended in 50 μL of a primary mAb (hybridoma supernatant or suboptimal dilution of purified antibody) or conjugate for 15 minutes at 4° C. in the dark, followed by a wash step (200 μL EDTA/FACS buffer, 1300 rpm centrifuge for 3 minutes). Secondary mAb were then added and incubated and cells were washed twice. For intracellular staining, cells were washed once in cold PBS then fixed and permeabilised using the BD Cytofix/Cytoperm kit (BD Biosciences, USA) according to the manufacturer's instructions. Cells were stained with 50 μL of primary antibody diluted in PermWash buffer for 30 mins, then washed in PermWash buffer and incubated with secondary antibody where required. Cells were washed in PermWash, then finally washed in FACS buffer, before being resuspended in 200 μL of FACS or EDTA/FACS buffer for acquisition.

Primary antibodies and lectins used were FITC-conjugated UEA-1 lectin (Silenus?), Ki67 (BD), PE-conjugated Ly51 (clone 6C3, Pharmingen) Ki67 (BD), I-A/I-E (BD), CD45 (LCA)(BD), PerCP Cy5.5 conjugated CD45 (BD), APC-conjugated I-A/I-E (Miltenyi), biotinylated CD31 (BD), and EpCAM (G8.8a, prepared in-house from a clone kindly donated by Dr. A. Fan) MTS15 hybridoma supernatant (prepared in-house), rat anti-mouse Aire (a kind gift from Dr. H. Scott). Secondary reagents used were FITC-conjugated anti-rat IgG2c, Streptavidin PerCPCy5.5 and Streptavidin APC.

A FACScalibur and CellQuest software (BD Biosciences, USA) were used for flow cytometric analysis, using four fluorescent channels (FL1 for FITC, FL2 for PE, FL3 for CyChrome/PECy5, PerCP and PI, and FL4 for APC). All compensations were performed on single colour labelling of an appropriate cell sample containing both positive and negative populations. Exclusion of non-viable and haemopoietic cells was based on forward light scatter versus side light scatter and CD45 expression respectively.

### Statistical Analysis

An unpaired, two-tailed Mann-Whitney Rank Sum U test was used to determine statistical significance between non-parametric data. A p-value of <0.05 was considered to be statistically significant.

### Results

### Effect of Thymus Depletion Models on T Cells

The relative loss and subsequent recovery of the thymus after damage with the immunosuppressant Cyclosporine (CsA), and the chemotherapeutic agents Cyclophosphamide (Cy) and Dexamethasone (Dex), was examined in order to find which epithelial subsets recovered first. All three drugs have decidedly different modes of action: cyclophosphamide is an alkylating agent which cross-links DNA nucleotides, dexamethasone is a glucocorticoid commonly used as an anti-inflammatory drug, and cyclosporine is a calcineurin inhibitor which prevents IL-2 production by blocking the NF-AT pathway.

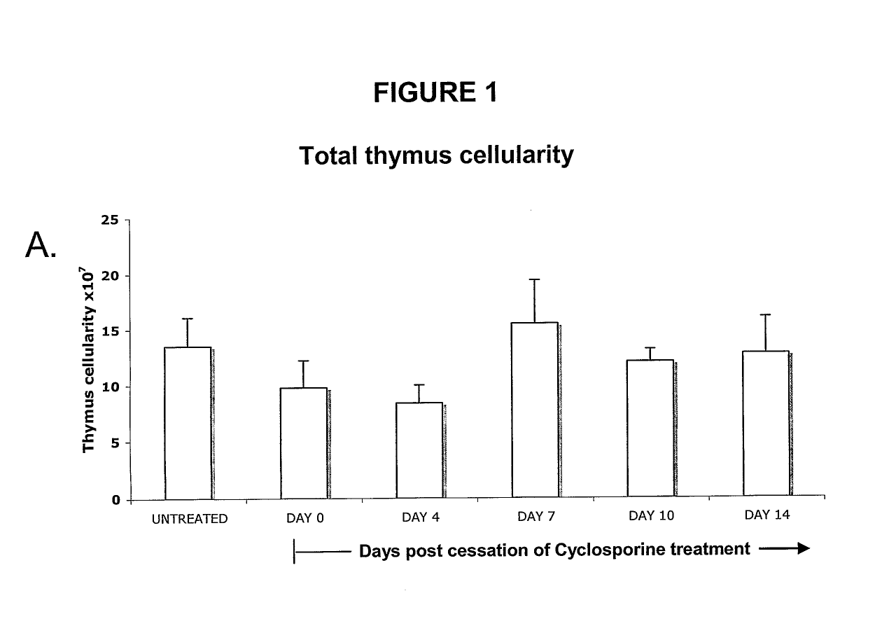

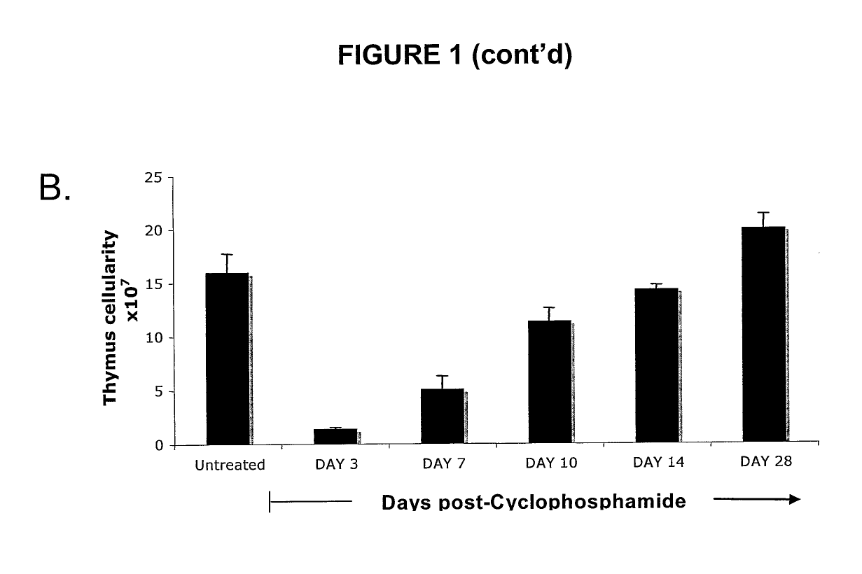

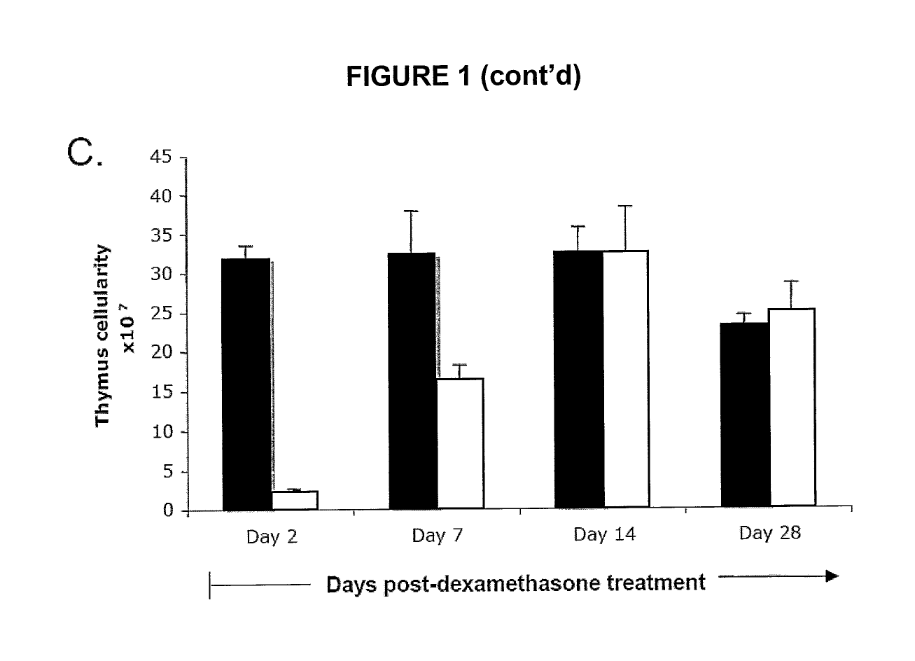

Thymocyte number was significantly reduced by all three treatments (FIG. 1). CsA-treated thymi showed the smallest degree of involution because it mainly effects activated cells these being primarily the maturing CD4+ CD8- and CD4-CD8+ cells of the medulla, which are ˜12-15% of the thymus. CD4+ and CD8+ single positive (SP) T cells, are reliant on IL-2 for their maintenance. CD4+ CD8+ double-positive (DP) T cells are immature, found in the cortex, and represent ˜80-85% of total thymocytes. These T cells are affected to a lesser extent by CsA, and recovered by day 7 post-treatment, while SP T cells were fully recovered by day 14 (data not shown).

Cy (FIG. 1B) and Dex (FIG. 1C) cause a marked reduction in all thymocyte subsets including the CD4+ CD8+ DP. These immature cells are very sensitive to cytotoxic agents as they are a more rapidly dividing population and more easily programmed to undergo apoptosis easily.

In all three models, the thymocytes have regenerated to normal levels, if not higher, by 14 days.

Effect of Thymus Depletion Models on Epithelial Cells

### (a) CsA

A contraction of the thymic stromal compartments has also been observed to occur as a consequence of chemotherapy and dexamethasone but it has not been clear whether this was more an “apparent” effect due to collapse of the thymic architecture from the loss of lymphocytes, or to a direct loss of the stromal cells. It is now shown that the thymus depletion models do effect the epithelial cells to different degrees.

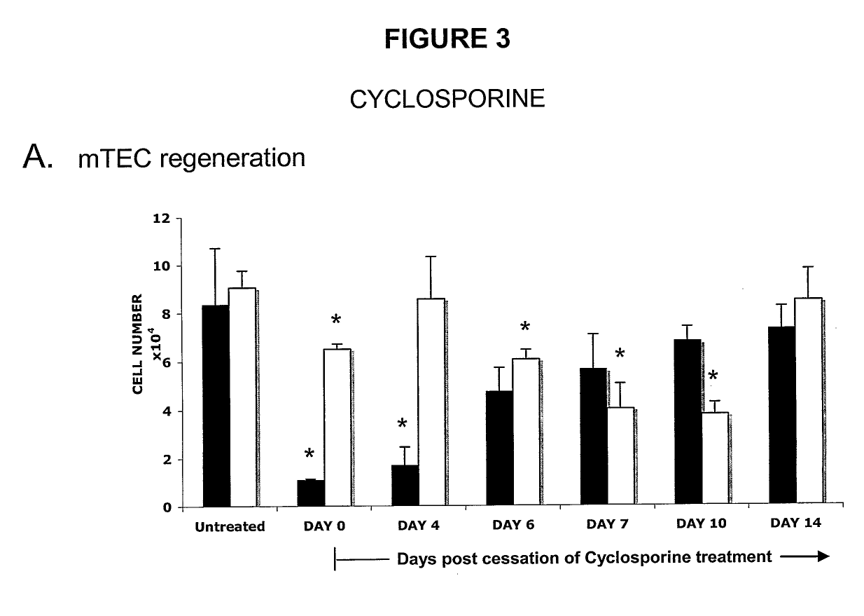

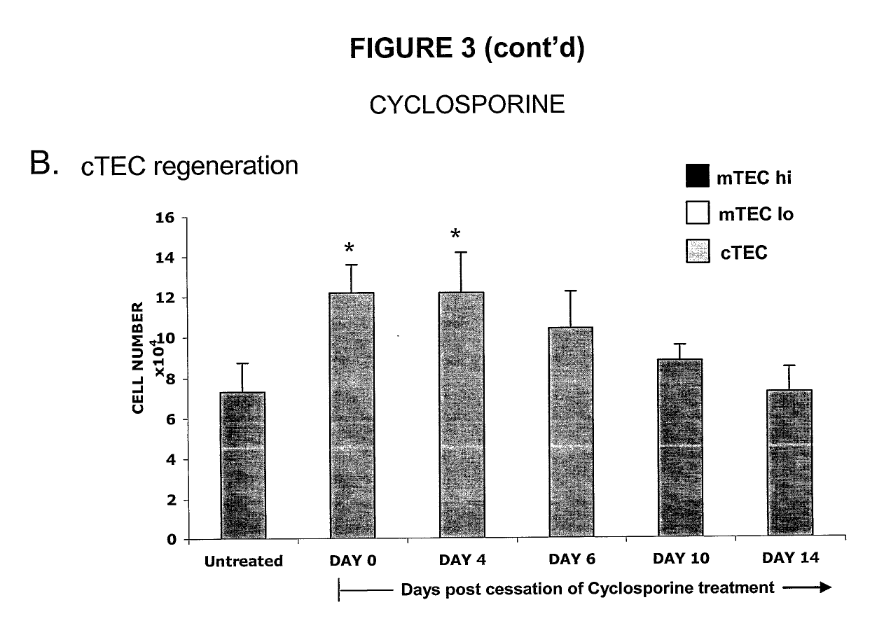

For CsA, both mTEC hi and m-TEC Lo but not cTEC, are reduced by CsA (FIG. 3A) consistent with the preferential loss of medullary thymocytes. This mTEC loss may thus be either by direct damage or through interruption of normal lymphostromal-T cell symbiosis (REF).

This model allowed examination of the selective recovery of the medullary epithelial cells.

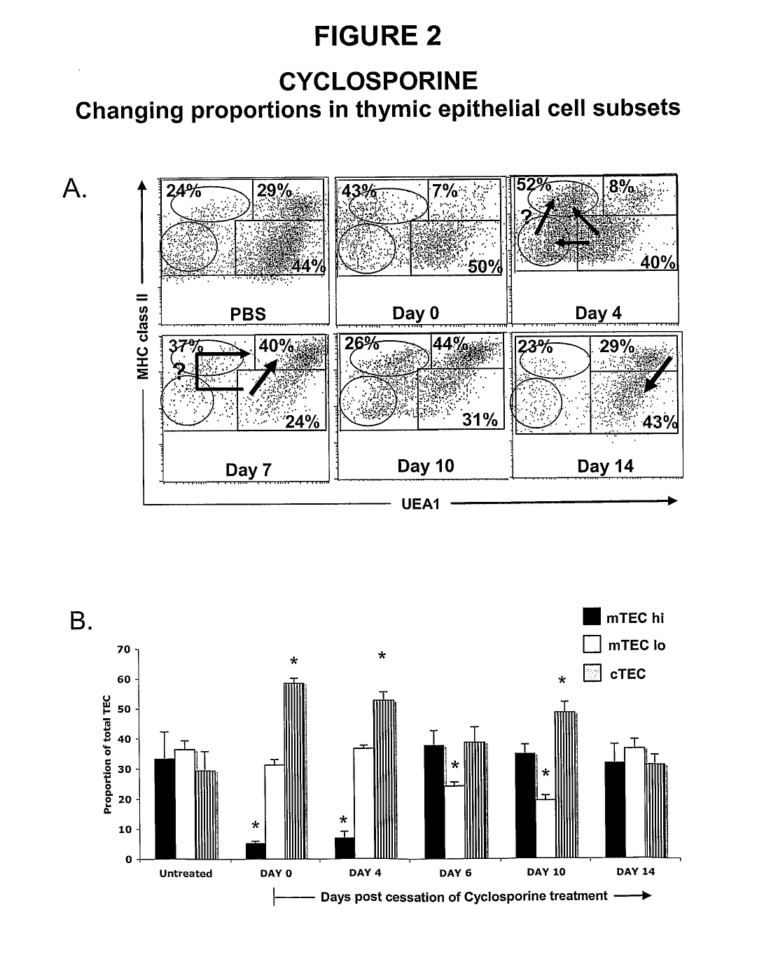

In CsA-treated mice, although the overall thymic cellularity was reduced by a third, the TEC loss was almost entirely limited to a fourfold reduction in the mTEC high subset which resulted in a proportional increase in the cTEC^{hi} cells. There was a noticeable population shift involving upregulation of MHC class II between days 4 and day 7. Regeneration of the cTEC occurred first. By day 4, the putative TEPC (MHC II lo, UEA1 lo) appeared to shift towards MHC II lo UEA1neg (cTEC Lo), and then to MHC II hi, UEA1 neg (cTEC Hi). From the FACS profiles which show a transition in staining (a smearing suggesting lineage progression) from mTEC-Lo to cTEC-Hi, it is also possible that some mTEC-Lo cells directly differentiated to cTEC Hi. By day 7, mTEC Hi cells began to recover, again apparently at the expense of the mTEC Lo subset because of the transition in staining. There was, however, also a reduced proportion of cTEC-Hi cells, and indeed total number of cTEC (FIG. 3B) between days 4 and 10, suggesting they may also contribute to the mTEC-Hi recovery (FIG. 2A, B). Finally, between days 10 and 14, there was a recovery of the mTEC-Lo cells—again because of the transitional nature of the FACS staining we propose these are derived from the mTEC-Hi cells. It is possible that some mTEC-Lo are derived by self renewal although there was no evidence of significant proliferation of these cells at any time-point.

By day 14, both mTEC stromal subsets had recovered and all TEC subsets were proportionally normal.

These proportional shifts were also reflected in the total numbers of mTEC (FIG. 3a) and cTEC (FIG. 3B) subsets.

Collectively these data from CsA-treated mice, indicated that both cTEChi and mTEChi cells i.e. the mature medullary and cortical epithelial cells were being generated de novo from mTEClo cells. However, it was possible that in response to CsA, the mTEChi cells were simply downregulating MHC class II. This did not appear likely for most of the mTEC-Lo cells at least, however, since populations of remaining mTEChi cells still showed normal MHC expression, and indeed, analysis of overall numbers of mTECs confirmed that downregulation of MHC could not account for the striking loss of mTEChi cells and there was no concomitant increase in mTEC-Hi cells (FIG. 3A). Numerically the mTEClo cells were only marginally reduced at the time of cessation of CsA and had fully recovered by day 4. There was then a progressive numerical loss of mTEC-Lo between days 4 and 10, consistent with the proposed progression (see above) of these cells initially to c-TEC-Lo and possibly cTEC-Hi, and finally to mTEC-Hi

It is possible that the mTEClo population either simply upregulates MHC class II as a homeostatic mechanism following loss of mTEChi, or that these cells are differentiating into mTEChi cells, which self-renew, and then replenish the mTEClo population. Such a process would fulfil the description of a transit-amplifying progenitor cell subset.

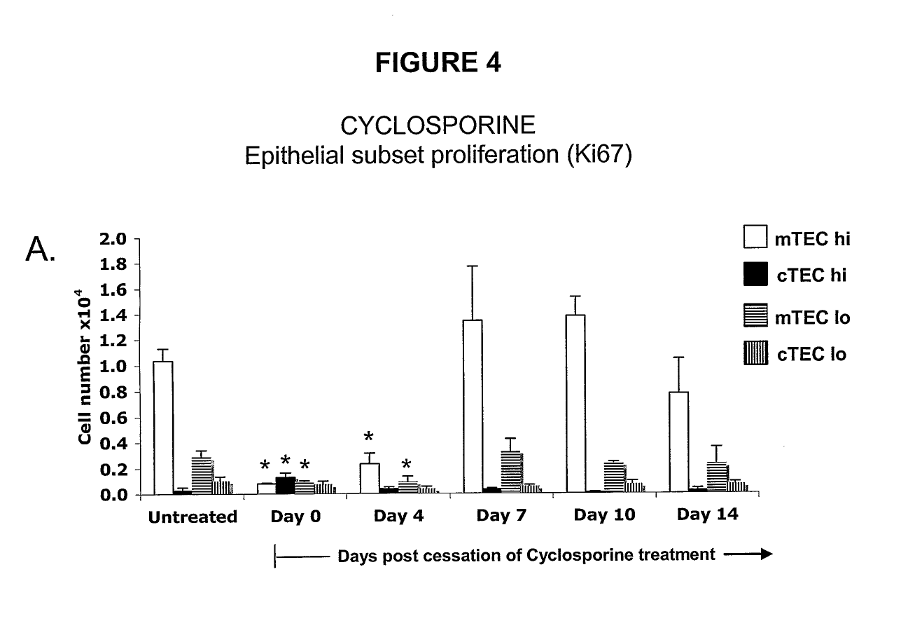

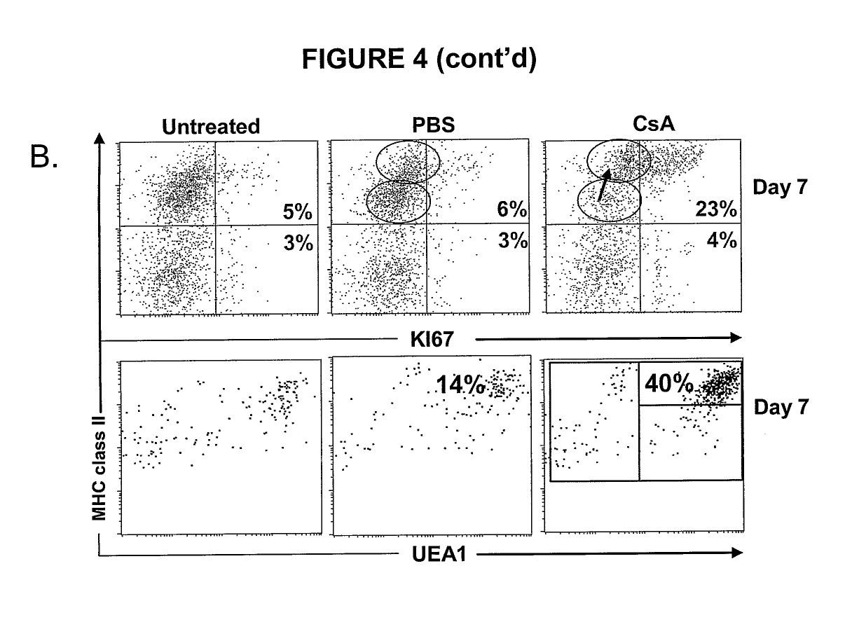

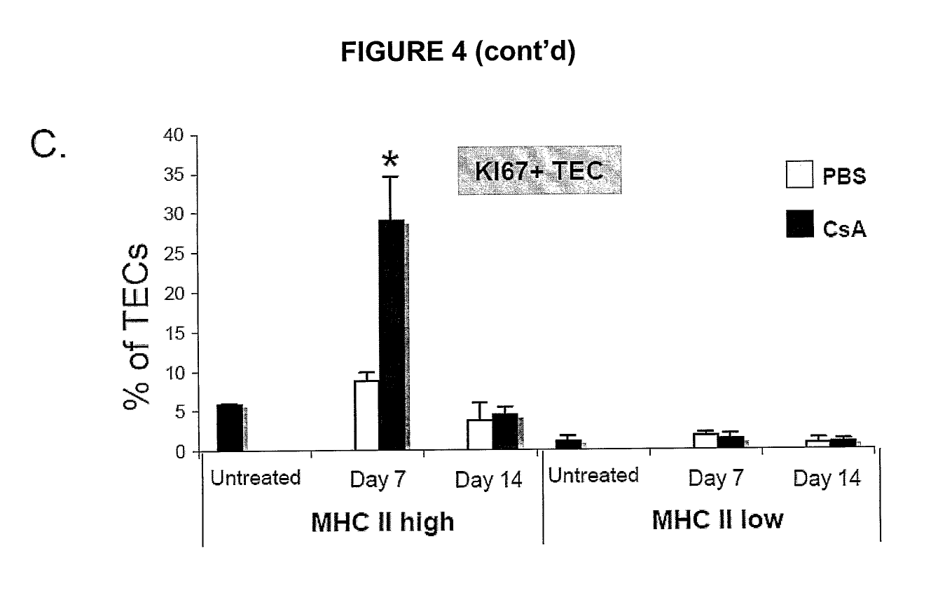

While the recovery of the mTEC-Lo population appeared from the FACS profiles to occur from the mTEC-Hi cells, there was no numerical loss of the mTEC-Hi cells. The question thus arose as to whether the latter were maintaining their numbers by proliferation. This was examined using Ki67 which is expressed on dividing cells. FIG. 4A clearly shows that mTEC-Hi cells are the major proliferating cell population in both the normal untreated thymus, (again consistent with why these cells are most sensitive to CsA) and the recovering thymus. Approximately 5% of TEC (i.e. MHC II+, CD45neg cells) are dividing in the normal thymus. By day 7 post CsA treatment there is a shift in MHC II lo to MHC II hi and many of these are now dividing (˜23% of total TEC are dividing) and have mTEC phenotype (UEA1+). MHC II lo cells rarely divide.

It was also necessary to confirm the extent of mTEC maturation, by examining the expression of the Aire gene, (Aire is only expressed in MHCII hi mTECs). A tight ratio of Aire+ to Aire− mTEC exists in normal thymi. The lineage of Aire+ mTEC is in itself highly contentious, and although it has been suggested that these cells split from the normal mTEC lineage during embryonic development, it is more widely held that Aire+mTEC are closely related to, and develop directly from, mTEC-Hi cells.

If the mTEC-Lo population merely upregulated MHC class II until the mTEC-Hi cells developed from a separate lineage, Aire expression should lag and a normal ratio of Aire expression would not be expected until just prior to mTEC-Hi recovery. If, however, the mTEC-Lo cells are a transit-amplifying population which sit static and turn over slowly until required to repopulate the active mTEC-Hi subset, the Aire ratio should recover early and quickly simultaneous with the appearance of mTEC-Hi cells. In addition, if this hypothesis is correct, mTEC-Hi cells should show obvious proliferation as soon as the mTEC-Lo numbers begin to wane.

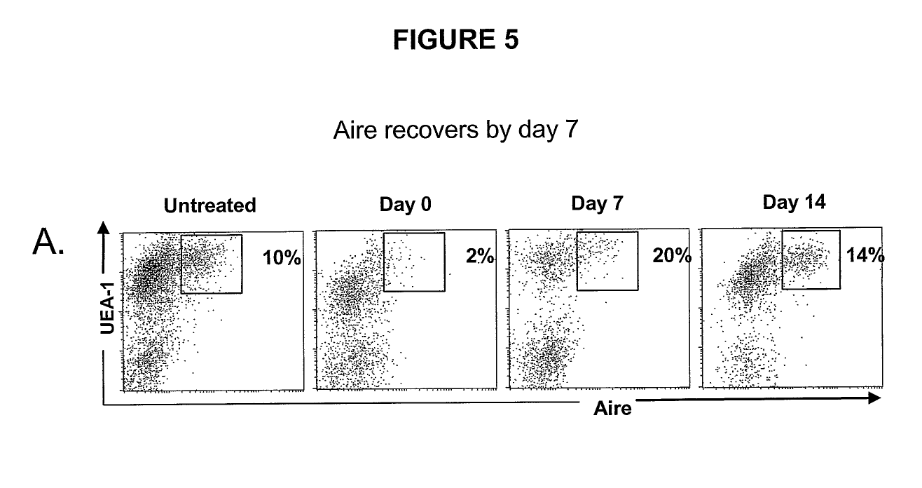

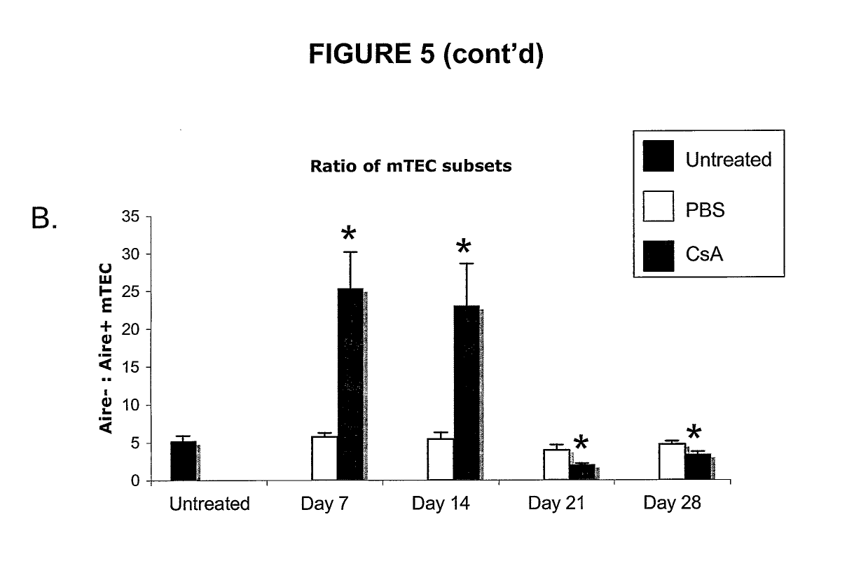

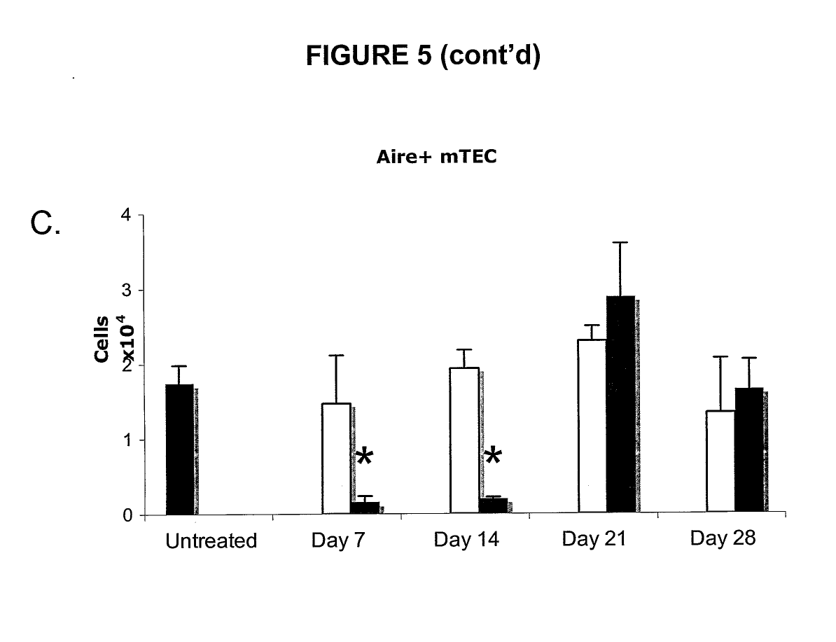

It was found (FIG. 5) that Aire+ mTECs are present at an even increased ratio by day 7, and that the mTEC-Hi cells are proliferating, strongly shown by Ki67 staining, at this timepoint.

The mTEC-Hi cells are thus initially formed from mTEC-Lo and then expand in number by proliferation.

Proportionally, again the most effected population was the mTEC-Hi

### (b) Dexamethasone

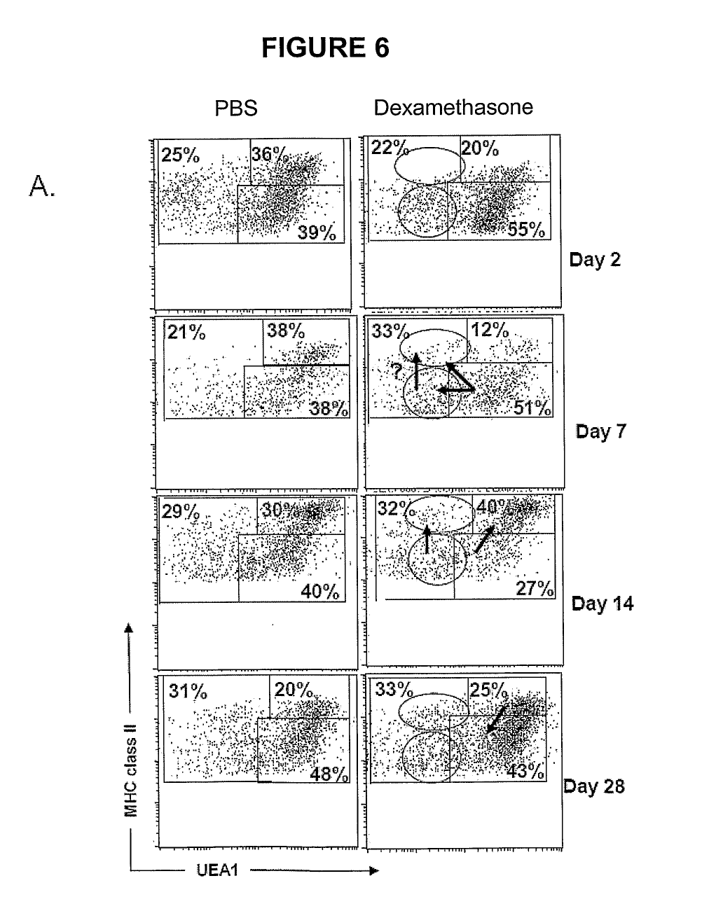

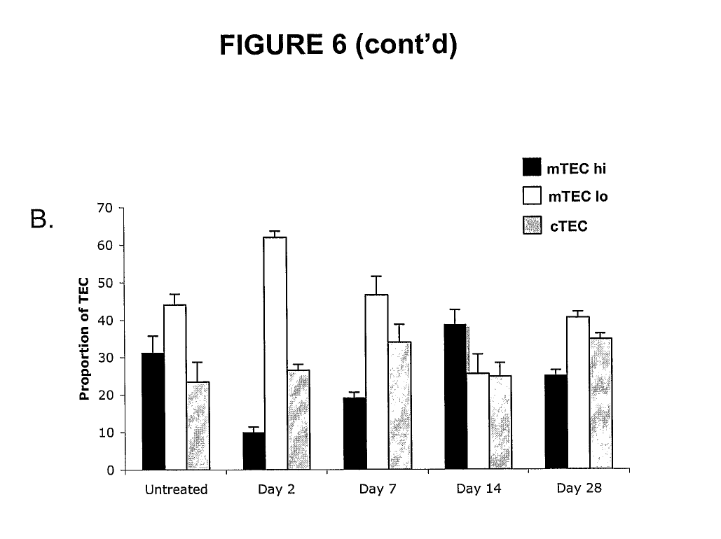

Proportionally, again the most effected population was the mTEC-Hi, with the effects on, and recovery of, the cTEC and mTEC-Hi showing similar trends to that observed for CsA (FIG. 6). mTEC-Lo were proportionally least effected by the treatment. By day 7 the cTEC were back to normal levels and their increase was paralleled by a decrease in mTEC-Lo cells; some cTEC may have also derived from upregulation of MHC class II on the pre-existing cTEC-Lo. The mTEC-Hi had recovered by day 14 again at the apparent expense of the mTEC-Lo cells

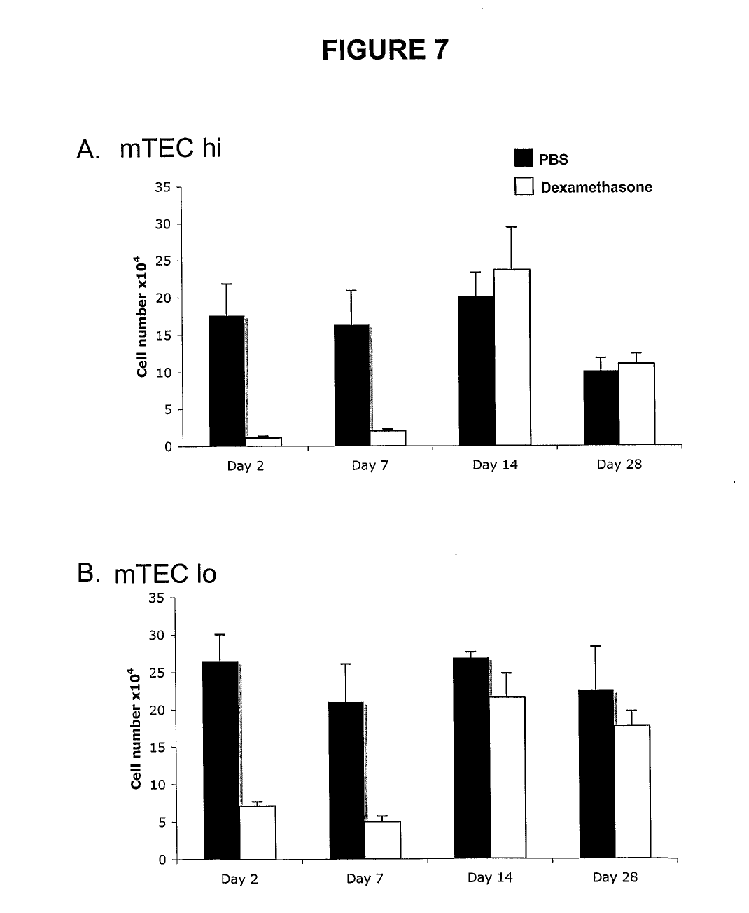

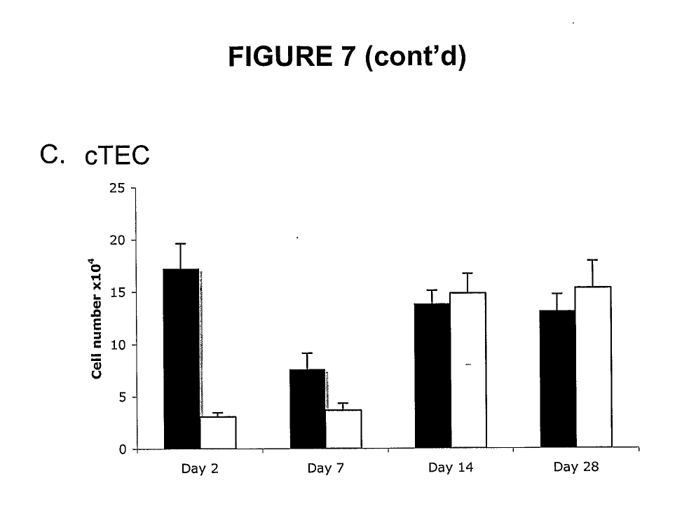

Numerically Dex clearly depleted the TEC, severely reducing all subsets—mTEC-Hi, mTEC-Lo and cTEC (FIG. 7). All of these had recovered by day 14 post-cessation of treatment. In this model, there was a major reduction in mTEC-Lo as well, differing from CsA treatment. The thymus is therefore expanding all subsets within a similar time span, —including thymocytes which are resuming development from all stages albeit in reduced numbers. In this case, the need for rapid recovery of mTEChi cell numbers, as for CsA is far less, because the cortical thymocytes were damaged too and they needed to be regenerated before the medullary SP's. This is unlike CsA where appreciable numbers of SP thymocytes may have been developing from post-positive selection and entering the medulla, as soon as 24 hours after withdrawal of treatment.

### (c) Cyclophosphamide

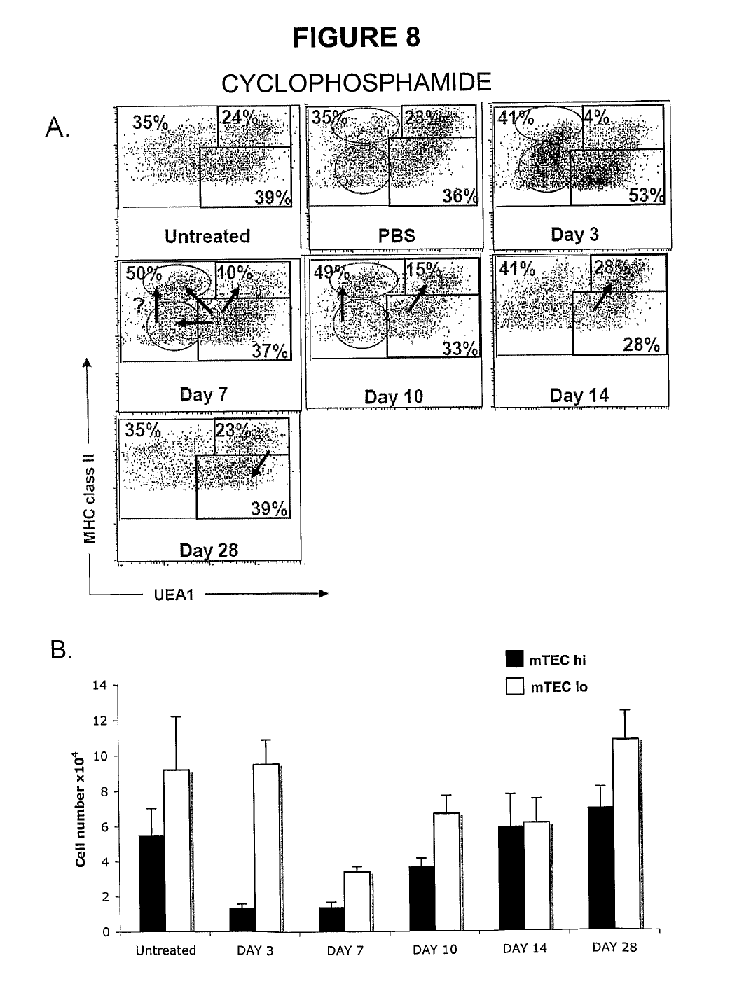

Cyclophosphamide causes apoptosis of rapidly dividing cells following DNA alkylation. In the thymus this involves the DP and SP thymocytes (data not shown) Again mTEC-Hi were proportionally the major subset effected (FIG. 8), consistent with them being the rapid-turnover TEC subset. mTEC-Hi were also the most effected numerically; mTEC-Lo cells were unaffected at day 3 but were also reduced numerically around day 7, after which time (day 10) the mTEC-Hi were beginning to recover. This is again consistent with mTEC-Lo being the progenitors for mTEC-hi cells. mTEC-Lo then increased to untreated levels by day 28.

### Aged Induced Thymic Atrophy

A paradoxical feature of the thymus is that despite its profound importance for establishing and maintaining immune competence, it undergoes severe atrophy particularly from the time of puberty. It has been shown that this effects all thymocyte and stromal cell subpopulations numerically. Ablation of sex steroids allows a comprehensive restoration of the thymus involving all cells and culminates with an increase in generation of naïve T cell export to the periphery.

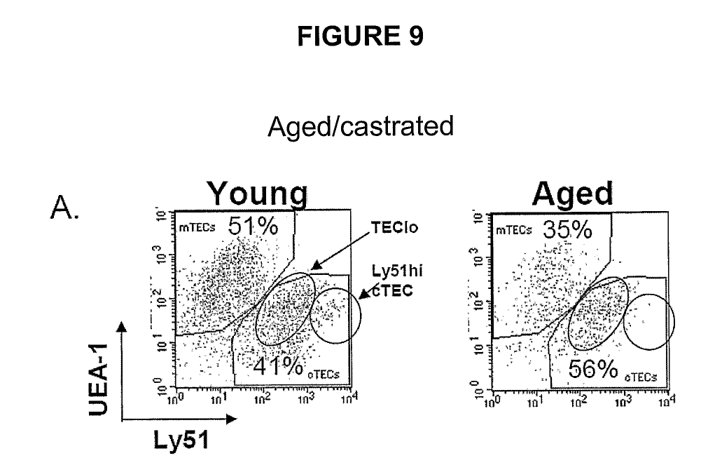

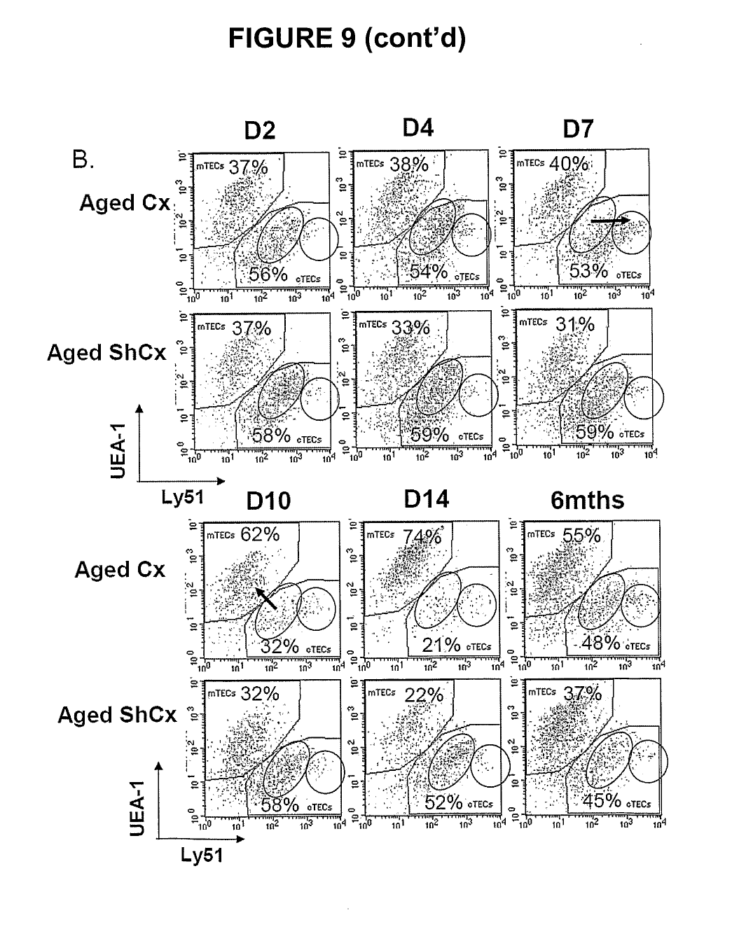

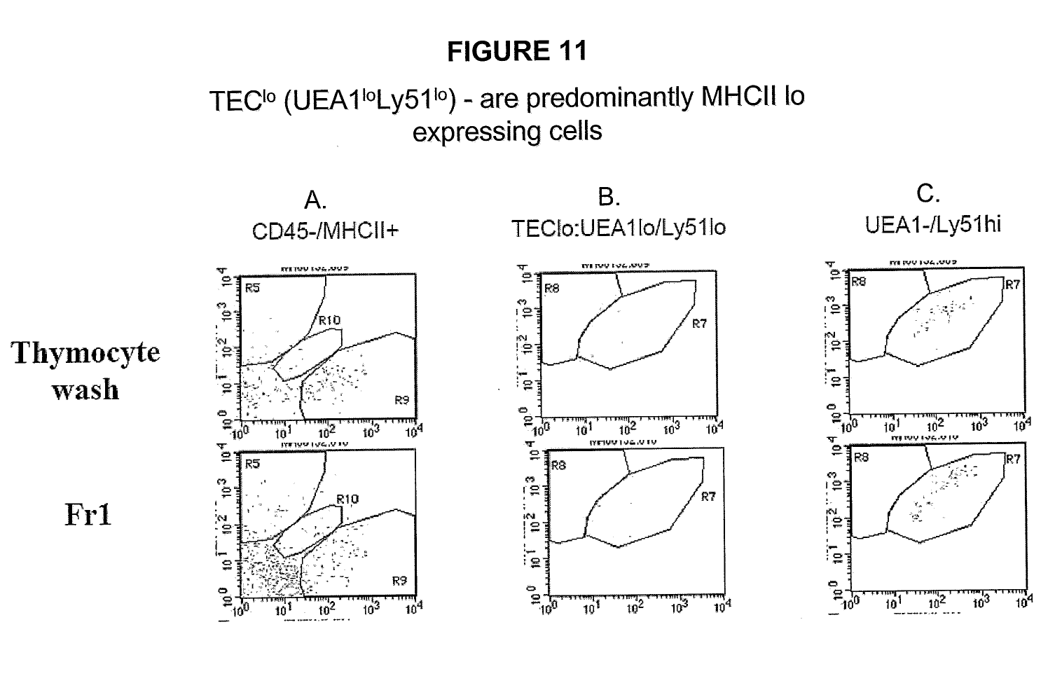

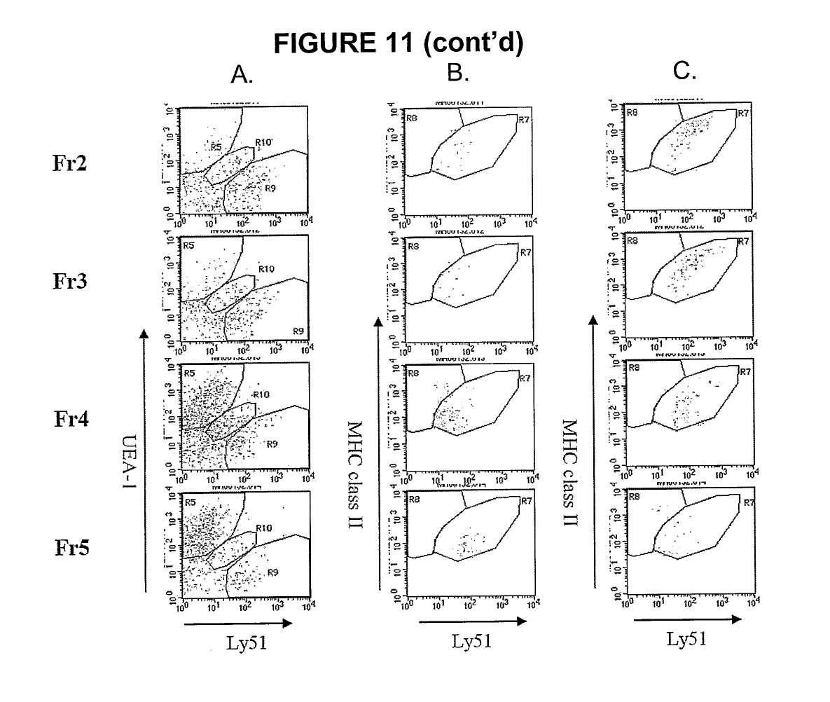

In the aged thymus there was a proportional decrease in mTEC and a corresponding increase in cTEC (FIG. 9). However there was also a decrease in Ly51/6C3-Hi cTEC, which we have shown to be MHC II-Hi (FIG. 11) with age. From day 7 post castration the cTEC-Hi cells returned with a concomitant loss of cTEC-Lo's. From day 10 associated with a loss in mTEC-Lo cells. From day 10 there was an increase in mTEC with further loss of the cTEC-Lo.

### Differential Gene Expression Profiles of TEClo Cells

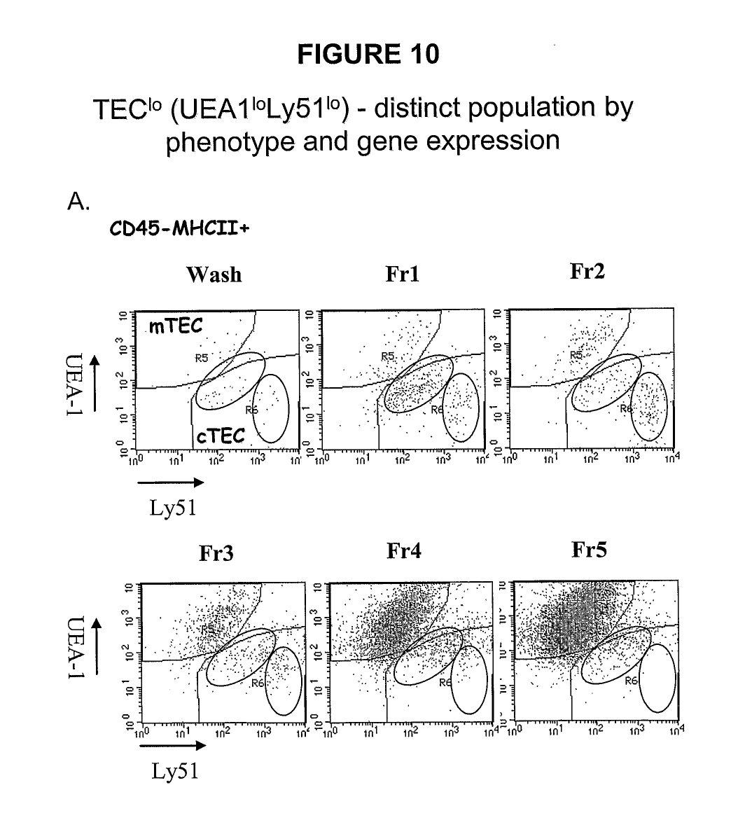

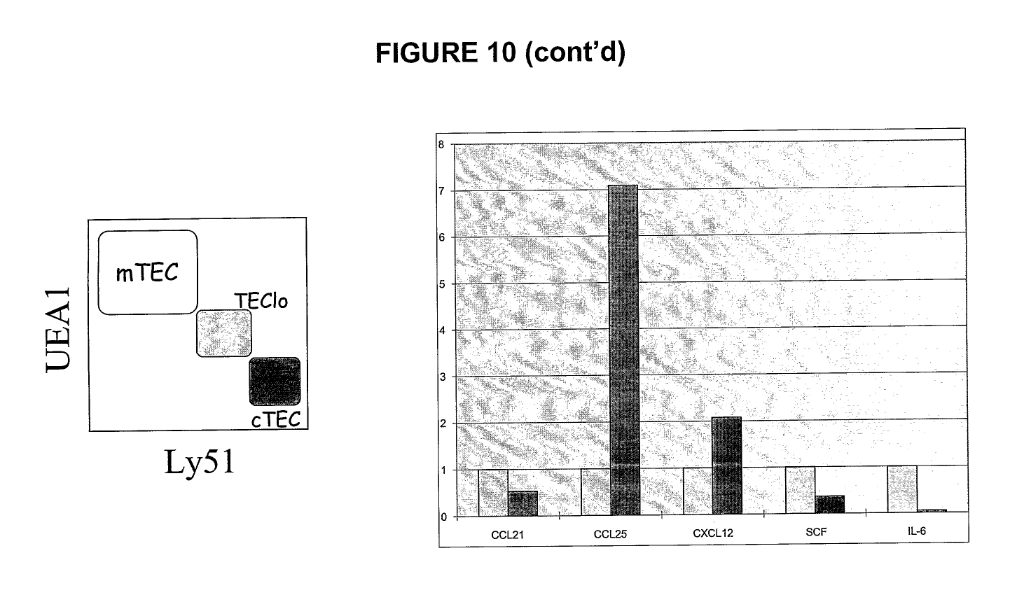

TEClo and cTEC Ly51hi epithelial cell subsets were purified and analysed for expression of various genes known to be expressed in the thymus (FIG. 10). The two populations show differential expression of these genes, confirming that they are a separate population of cells.

The results have arisen from studies on the involution and regeneration of TEC populations following treatment with drugs known to deplete the thymus. The effects of chemotherapy (cyclophosphamide), corticosteroids (dexamethasone), and the immunosuppressive drug cyclosporin A (CsA) have been examined. All of these have are known to cause variable depletion of thymus lymphocytes. Using cyclophosphamide and CsA it was demonstrated that TEC as a population are significantly reduced whereas non-TEC stromal cells were not effected. Both mTEC^{hi} and to a lesser extent cTEC^{hi} were depleted, whereas the mTEC^{lo} were relatively unaffected. It was found that the cTEC^{hi} population recovers first followed by the mTEC^{hi} cells, both doing so at the expense of maintenance of the mTEC^{lo} population, suggesting a lineage relationship. The data are consistent with the proposal that mTEC^{lo} cells (most likely also UEA1 Lo and Ly51 Lo) are a resting population that acts rapidly after damage to upregulate MHC class II and proliferate, preferentially restoring the cTEC^{hi} then mTEC^{hi} population. These cells then downregulate MHC class II to repopulate the MHC class II^{lo} subsets. These observations fulfil the functional requirements of a transit amplifying population. A transition of cells differentiating from TEC^{lo} to cTEC^{Hi} and then mTEC^{Hi} for all the models of repair of thymic damage, including the reversal of age-induced thymic atrophy, by inhibition of sex steroids has also been found.

### Example 2

### Materials and Methods

### Animals

Male and female C57B1/6J (B6) mice aged at 8-12 weeks were obtained from Monash Animal Services and housed under SPF conditions, in accordance with institutional guidelines. Experiments were performed under approval from the Monash University Animal Ethics Committee.

### Chemotherapy

In all experiments, the final day of treatment was designated as Day 0. Mice were then humanely killed by CO₂ asphyxiation at indicated timepoints. Mice were given 15 mg/kg/day Cyclosporine A (Novartis, Australia) i.p. in PBS for 7 or 14 days, as indicated; or 100 mg/kg/day of Cyclophosphamide (Pharmacia, Australia) i.p. in PBS for 2 days; or a single injection, at 20 mg/kg, of Dexamethasone (Lyppards, Australia).

### Individual Thymus Digestion for Flow Cytometric Analysis

This procedure was performed as previously described (Gray et al. 2008, J Immunol Methods 329:56-66). Briefly, thymi were agitated in RPMI-1640 to flush out thymocytes. After collecting supernatant, thymic fragments were incubated individually at 37° C. for 10 minutes in 2 mL of 0.125% (w/v) Collagenase D with 500 μL of 0.1% (w/v) DNAse I (both from Roche, Germany) in RPMI-1640, with regular agitation. The supernatant was collected, kept on ice, and the digestion repeated three times, followed by a final 10 minute digestion in 1 mL of 0.125% (w/v) Collagenase/Dispase (Roche, Germany) with 500 μL 0.1% (w/v) DNAse I in RPMI-1640. The supernatant fractions were then pooled and centrifuged, keeping each thymus separate, at 472 gₘₐₓ for 5 minutes, and resuspended in cold EDTA/FACS buffer (5 mM EDTA in PBS with 2% FCS and 0.02% Sodium Azide). Cells were filtered through 100 μm mesh. Cell counts were performed using a Z2 Coulter Counter (Beckman Coulter USA).

Pooled Thymus Digestion for Cell Sorting and mRNA Analysis

This procedure was performed as previously described (Gray et al. 2008, supra). Ten thymi were pooled, agitated several times in 10 ml RPMI-1640, and the supernatant collected. Following this, collective digestion of the pooled thymi proceeded as described above, using 5 ml of 0.125% (w/v) Collagenase D and 500 μl of 0.1% (w/v) DNAse I for 10 minutes per digestion step. Cells from all fractions were filtered and counted as described above.

### Immunofluorescent Staining and Flow Cytometry

Cells (5×10⁶) were dispensed into 96 well round-bottom plates and resuspended in 50 μL of titrated primary mAb (hybridoma supernatant or purified antibody) or conjugate for 15 minutes at 4° C. in the dark, followed by a wash step. Secondary mAbs were then added, and after incubation cells were washed twice. For intracellular staining, surface-stained cells were washed once in cold PBS then fixed and permeabilized using the BD Cytofix/Cytoperm kit (BD Biosciences, USA) according to the manufacturer's instructions. Cell staining was, however, adapted from this protocol. Cells were stained with 50 μL of primary antibody diluted in PermWash buffer for 30 mins, then washed once in PermWash buffer and incubated with secondary antibody. Cells were washed once in PermWash, then finally washed in EDTA/FACS buffer, before being resuspended in 200 μL of EDTA/FACS buffer for acquisition.

A FACScalibur and CellQuest software (BD Biosciences, USA) were used for flow cytometric analysis, using four fluorescent channels (FL1 for FITC, FL2 for PE, FL3 for CyChrome/PECy5, PerCP and PI, and FL4 for APC). All compensations were performed on single colour labelling of an appropriate cell sample containing both positive and negative populations. Exclusion of non-viable and haemopoietic cells was based on forward light scatter versus side light scatter and CD45 expression respectively. Statistical analysis was performed using SPSS v.15.0 software.

### Cell Sorting for Molecular Analysis

Following pooled thymus digestion, CD45-stromal cells were enriched to 70-90% using CD45 microbeads and AutoMACS depletion (both Miltenyi Biotech, Germany) using the ‘Depletes’ program and a protocol adapted from the manufacturer's instructions (Gray et al. 2008, supra). The negative fraction was stained with the appropriate immunofluorescence markers, and sorted on a FACSAria or FACSVantage cell sorter (BD Biosciences, USA) at a pressure not exceeding 30 psi. Cells were collected in 30% (v/v) FCS in RPMI-1640, washed in PBS, and lysed in RLT buffer (Qiagen, USA) before being snap frozen in liquid nitrogen and stored at −80° C.

RNA Preparation and cDNA Synthesis

Total RNA was isolated from sorted thymic stromal cells using the Qiagen Micro- or Mini-Kit, as per the manufacturer's instructions (Qiagen, USA). RNA was reverse transcribed using Superscript III first-strand synthesis system for RT-PCR (Invitrogen, USA) and oligo(dT) oligonucleotides according to the manufacturer's instructions.

### Quantitative PCR

For each validated primer (200 nM), qPCR was performed in 10 μl reactions using Platinum SYBR Green qPCR Supermix UDG (Invitrogen, USA) on a Corbett Rotor-Gene 3000 (Corbett Research, Australia). After an initial hold for 2 min at 50° C., the reaction was heated to 95° C. for 10 min, followed by 45 cycles of amplification at 95° C. for 15 sec and 60° C. for 60 sec. The Delta Delta CT (2^{-ΔΔCT}) method was used to calculate relative levels of target mRNA compared to GAPDH (31). Primers for p63 were sourced from Qiagen (Trp63; Catalogue number QT00197904).

### Immunohistology and Confocal Microscopy

Dissected organs were embedded in OCT compound (TissueTek; Miles Scientific, USA), frozen in a liquid nitrogen/isopentane slurry, and stored at −80° C. A TissueTek II cryostat (Miles Scientific, USA) was used to cut 8-12 μm frozen sections at −25° C. Slides were air-dried at 4° C. for 30 minutes and the section ringed with a wax pen (DakoCytomation, USA). Sections were stained at room temperature for 20 minutes with 50 μL of primary mAb (hybridoma supernatant or sub-saturating dilution of purified antibody) before being washed in PBS for 5 minutes, then stained for 20 minutes with secondary mAb, and washed three times in PBS. Slides were mounted with coverslips using fluorescent mounting medium (DakoCytomation, USA) and images were acquired on a Bio-Rad MRC 1024 confocal microscope with a three-line Kr/Ar laser (excitation lines 488, 568 and 647 nm) using LaserSharp acquisition software v.3.2 (Biorad, USA).

### Antibodies and Conjugates

Antibodies and conjugates were obtained from BD Pharmingen (USA) unless otherwise indicated. Primary antibodies and conjugates used were UEA1 lectin (Vector Laboratories, USA), anti-Ly51 (clones 6C3 and BP-1); anti-IA/IE (clone M5/114.15.2); anti-CD45 (clone 30-F11); anti-Aire (clone 5H12-2, a kind gift from Dr. Hamish Scott); anti-EpCAM (clone G8.8a, a kind gift from Dr. Andrew Fan); MTS24 (grown in-house); anti-Ki67 (clone B56); anti-CD80 (clone 1G11); anti-CD4 (clone GK1.5); anti-CD8 (clone 53-6.7); anti-TCRbeta (clone H57-597); anti-CD25 (clone PC61); polyclonal rabbit anti-bovine pan-cytokeratin (DakoCytomation, Denmark), anti-keratin 8 (Troma 1, a kind gift from Dr. Dale Godfrey); and polyclonal rabbit anti-mouse MK5 (Covance, USA). Secondary antibodies used were anti-rat IgG2c (Southern Biotech, USA), anti-rat IgG2a (Southern Biotech, USA) Streptavidin PerCP Cy5.5 and Streptavidin APC, anti-rabbit IgG-Alexa 488 (Invitrogen, USA).

### Statistical Analysis

An unpaired, two-tailed Mann-Whitney Rank Sum U test was used to determine statistical significance between non-parametric data. A p-value of <0.05 was considered to be statistically significant.

### Results

mTEC-lo Directly Seed mTEC-hi In Vivo After Thymic Damage

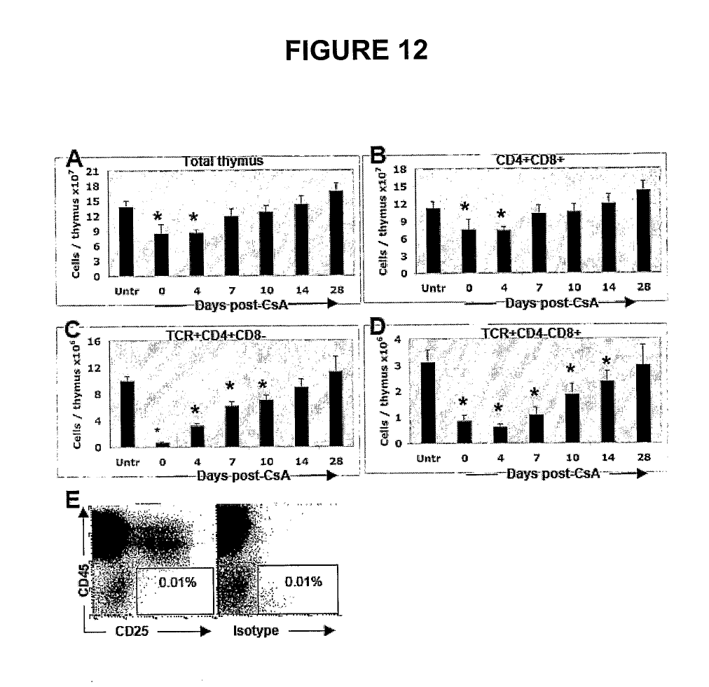

CsA caused a mild, transient thymic involution including a small reduction in CD4⁺CD8⁺ double positive (DP) thymocytes, but a severe loss of both CD4⁺ and CD8⁺ single positive (SP) thymocytes (FIG. 12). Consistent with the propensity to induce thymus-dependent autoimmunity, a loss in proportion and number of thymi cDCs and Tregs was found. This has been linked to the development of a thymic-dependent autoimmune disease following CsA withdrawal in some patients and animal models (Beschornere et al. 1991, Cell Immunol 132:505-514; Beschorner et al. 1987, Cell Immunol 110:350-364; Beschorner et al. 1991, Transplantation 52:668-674). CsA inhibits T cell activation through selective inhibition of calcineurin-mediated dephosphorylation of the nuclear factor of activated T cells (NF-ATc), which initiates transcription of IL-2 (Stepkowski, S. M. 2000. Expert Rev Mol Med 2:1-23). Thymic stromal cells do not express the IL-2 receptor (FIG. 12E), but given the intricate co-dependence of SP T cells and mTEC, it was hypothesised that, along with thymocytes, mTEC would also be selectively affected by CsA treatment.

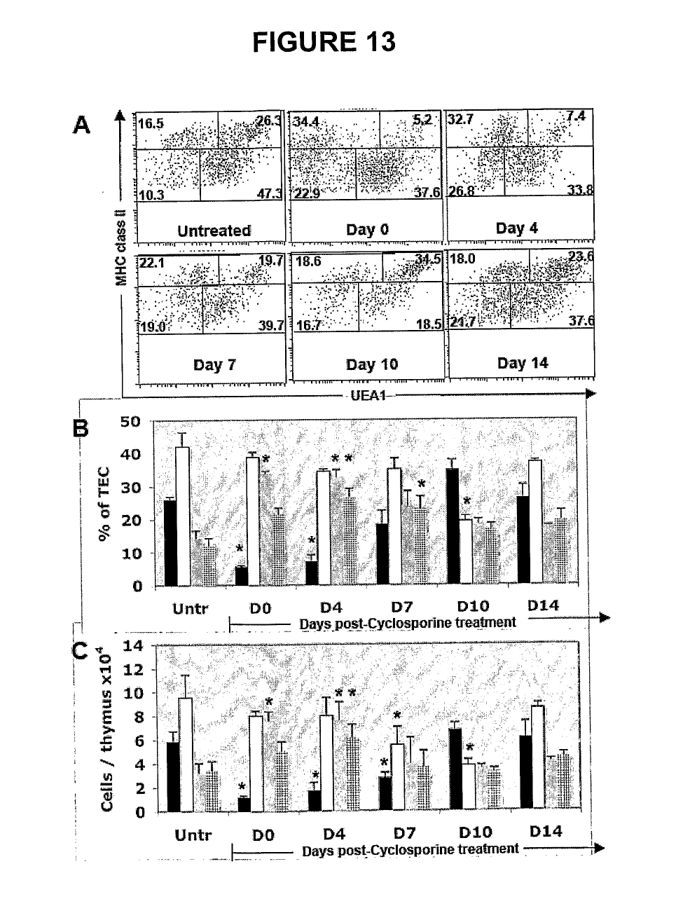

CsA treatment was found to selectively and severely reduce mTEChi proportion (FIG. 13A, B) and number (FIG. 13C), with recovery occurring by day 10 post treatment cessation. The cells were not merely downregulating MHC class II during treatment, since the number of mTEC-lo cells did not increase; nor downregulating UEA1, since 96-98% of the UEA1⁻ MHC class II^{hi} cells expressed the cortical marker Ly51, which is not expressed by mTEC-hi cells.

Most dramatically, the restoration of mTEC-hi was accompanied by a simultaneous loss of the mTEC-lo subset, with the flow cytometry profiles suggesting that these cells were upregulating MHC class II expression between days 4 and 7 (FIG. 13A). This was supported numerically (FIG. 13C). In fact, the total number of mTEC remained relatively constant through days 0, 4 and 7, during which the number of mTEC-lo reduced in direct proportion with the increase in mTEC-hi. By day 10, the total number of mTEC increased and returned to normal by day 14 post-treatment, indicating further replenishment of both the mTEC-hi and mTEC-lo subsets.

Unlike mTEC-lo, cTEC-hi and cTEC-lo subsets were increased in proportion and number during CsA treatment. This is very noteworthy and may indicate a role in thymic regeneration. Their return to homeostasis by day 7, prior to the large increase in mTEC-hi still supports the hypothesis that the mTEC-lo population, rather than cTEC, contains progenitors to directly seed mTEC-hi in vivo following thymic damage.

### Aire Expression is Preferentially Restored

As the only confirmed marker of terminal mTEC differentiation (Gabler et al. 2007, Eur J Immunol 37:3363-3372; Rossi et al. 2007, J Exp Med 204:1267-1272; Gray et al. 2007, J. Exp. Med. 204:2521-2528), Aire expression was examined during thymic recovery both for its clinical importance to immune recovery, and in order to assess whether mTEC-lo were truly differentiating, or merely upregulating MHC class II to restore some homeostatic balance, without functional gain of mTEC-hi-restricted traits, such as ectopic self-antigen expression.

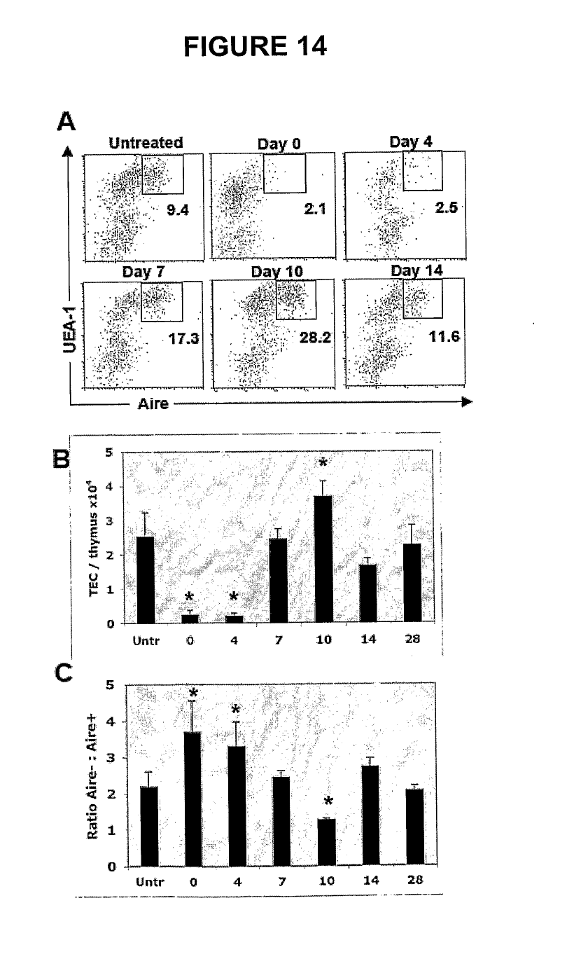

Aire expression was almost completely abrogated by treatment with CsA (FIG. 14A), as well as cyclophosphamide and dexamethasone. Even those cells remaining showed reduced fluorescence intensity (FIG. 14A), suggesting reduced protein expression. By day 4 after treatment ceased, normal intensity of Aire expression was restored, and at days 7 and 10 during recovery, significantly increased proportions of TEC expressed Aire. After 7 days, the number of Aire⁺ mTEC was fully regenerated. Interestingly, there was a significant increase in the number of Aire⁺ mTEC at day 10 (FIG. 14B). Since Aire⁺ mTEC develop from transit-amplifying Aire mTEC-hi (8-10), the ratio of these cell types was examined. Ordinarily, the ratio of Aire⁻ mTEC-hi to Aire⁺ mTEC-hi is 2:1. The proportion of mTEC-hi cells expressing Aire normalised by day 7, consistent with the fact that mTEC-lo cells were differentiating into functional (at least in terms of Aire expression) mTEC-hi cells. Furthermore, the proportion of mTEC-hi expressing Aire increased to a 1:1 ratio at day 10.

The mTEC-Lo Subset is a Large Pool of Resting mTEC-hi Progenitors

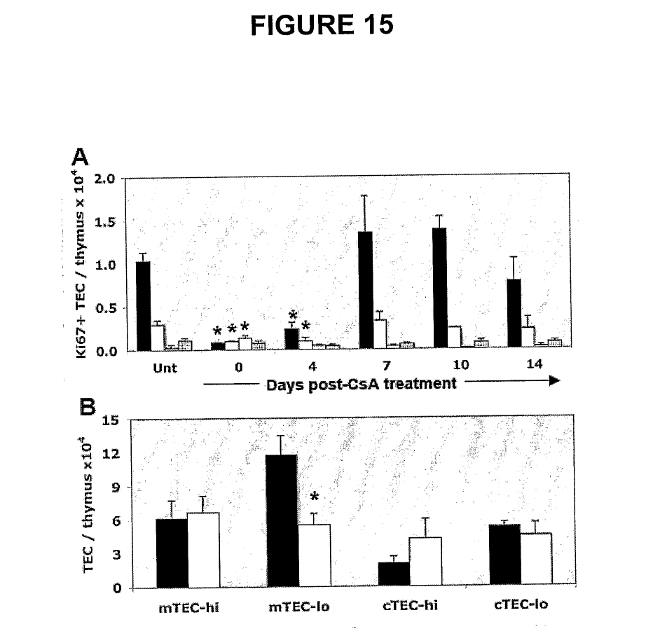

Using Ki67 expression, it was found that during thymic regeneration, mTEC-hi cells are always cycling at the highest proportion of TEC (FIG. 15A). This marker identifies cells that are not in G0, rather than directly indicating cell proliferation in total, such as in pulse-chase BrdU incorporation studies. It therefore provides a ‘snapshot’ approach whereby all currently proliferating cells are always Ki67⁺, making it a useful indicator in differentiation studies. The cTEC-hi population showed the earliest evidence of an increase in proliferating cells—at day 0, consistent with their early increase in number and proportion. However, as in untreated mice, the highest proportion of cells positive for Ki67 were MHC class II^{hi} and expressed UEA1. This demonstrated that the mTEC-lo subset was not proliferating ‘en masse’ prior to differentiation, suggesting that these cells initially differentiate post-damage, followed by self-renewal of transit-amplifying mTEC-hi cells, which may then replenish the mTEC-lo subset. True TESC, if within this subset, would be able to self-renew as well as regenerate TEC subsets.

The possibility still remained that the reproducibly coincident reduction in mTEC-lo and increase in mTEC-hi occurred because CsA damaged an unknown mTEC-lo precursor cell. This theory would require that mTEC-lo cells had a defined lifespan, and were reduced at days 7-10 due to non-replacement, before increasing at day 14 following replenishment after recovery of their precursor. To test this theory, the timecourse was varied and treated with CsA for 7, rather than 14 days.

Following 7 days of treatment, at day 0 and day 4, the TEC subset composition was indistinguishable from the thymic damage mediated by 14 days of treatment. However, mTEC-hi recovered slightly faster, and reached normal levels by day 7 (post-cessation of treatment). This was again accompanied by a coincident numerical loss of the mTEC-lo subset (FIG. 15B), suggesting that the reduction after 14 days of treatment is not simply due to mTEC-lo attrition at the end of their lifespan. Again, when mice were treated with CsA daily for 3 weeks, there was loss of mTEC-lo 7 days after treatment was stopped, and concomitant with mTEC-hi regeneration.

The mTEC-lo Progenitor Pool Expresses Markers of Embryonic TESC and Differentiated cTEC

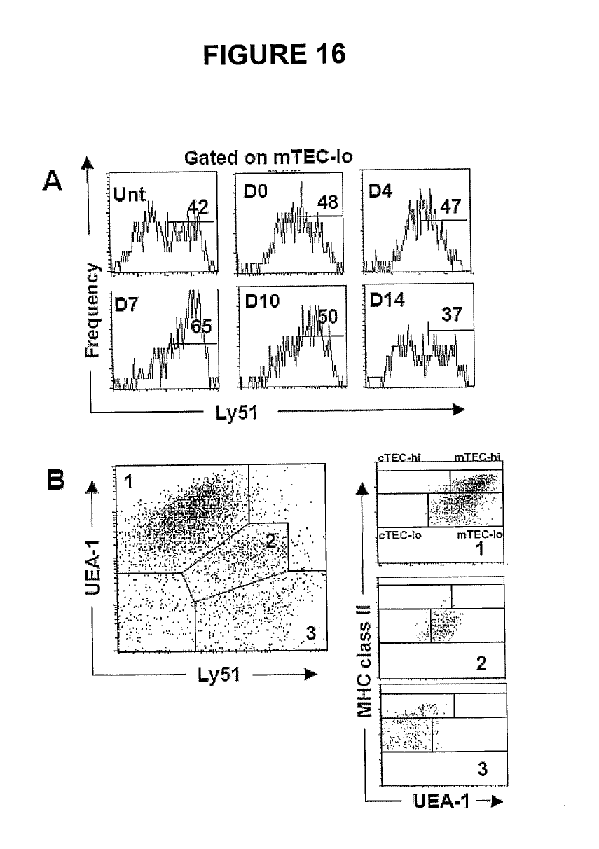

The cortical marker Ly51 is expressed by approximately 40% of mTEC-lo (FIG. 16A). At the time of CsA treatment withdrawal (Day 0), all mTEC-lo appeared to express Ly51. At day 7, the timepoint at which mTEC-lo cells begin to differentiate into mTEC-hi, the mTEC-lo Ly51^{lo/neg} population is lost, In untreated mice, analysis of UEA1 and Ly51 expression indicates 3 cell populations (FIG. 16B). This “middle” population expresses both UEA1 and Ly51 and clearly resides within the mTEC-lo gate in untreated thymi (FIG. 16B). Fewer than 10% of mTEC-hi expressed Ly51, and this proportion did not alter either during treatment or recovery. Together these data indicate that this “triple-lo” (MHC class II^{lo}, UEA1^{lo}, Ly51^{lo} population harbours the epithelial progenitor cell activated in response to thymic epithelial damage.

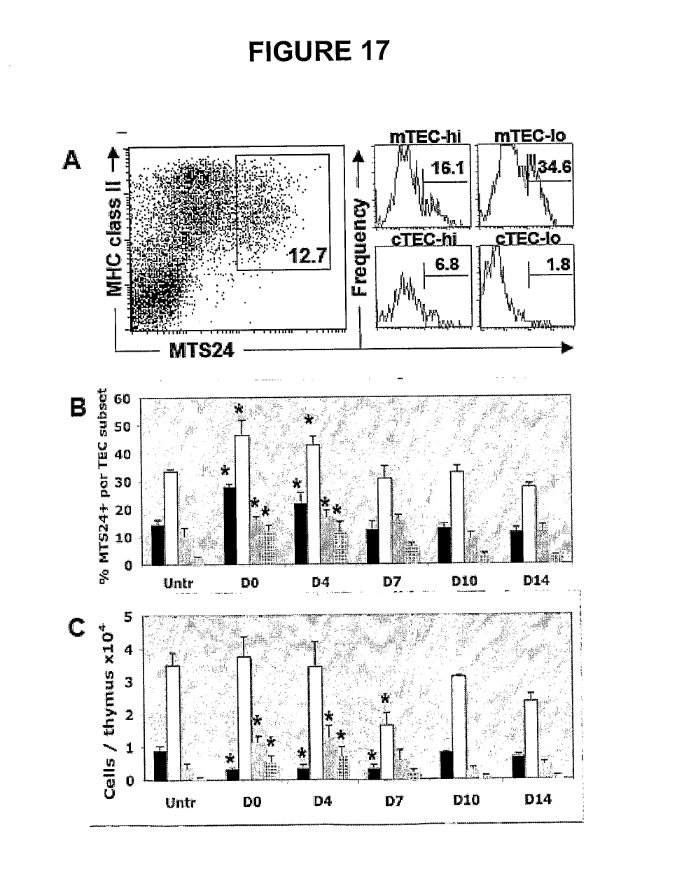

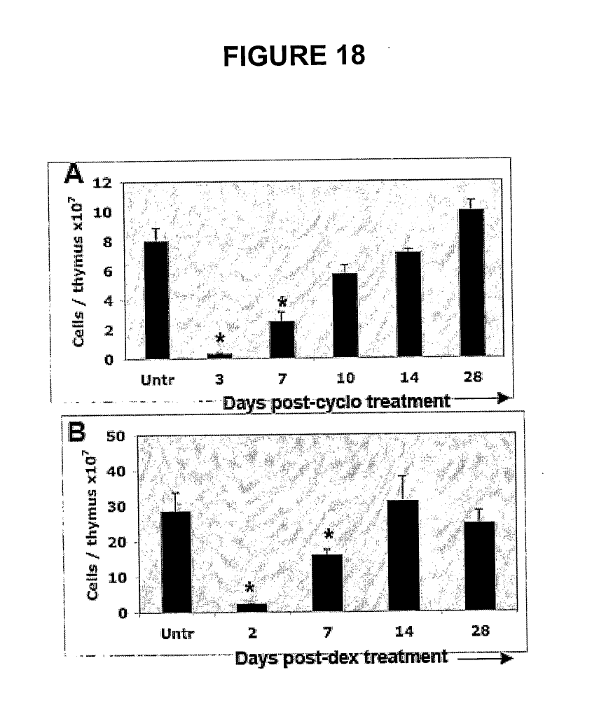

Given the heterogeneity of TEC and Plet-1 expression on high-efficiency pTESC in the early embryo, MTS24 staining was also examined as a reagent that could potentially identify a pTESC population. In the postnatal thymus, a large subset of TEC are stained by MTS24. The majority of these were mTEC-lo, although the antigen was expressed by all subsets to some extent (FIG. 17A). Interestingly, MTS24⁺ cells were both proportionally and numerically more resistant to thymic damage from CsA and cyclophosphamide than MTS24⁻ cells (FIG. 17B). In combination, the data are consistent with MTS24⁺ progenitor cells residing within the mTEC-lo subset which, upon differentiation to mTEC-hi, lose expression of the Plet-1 antigen. Furthermore, cTEC-hi and cTEC-lo MTS24⁺ cells increased in both proportion and number following CsA treatment (FIG. 17C), showing a broad upregulation of this antigen in response to thymic damage. All subsets showed normal expression by day 10 after treatment.

### Conserved Regeneration Patterns Across More Severe Models of Thymic Damage

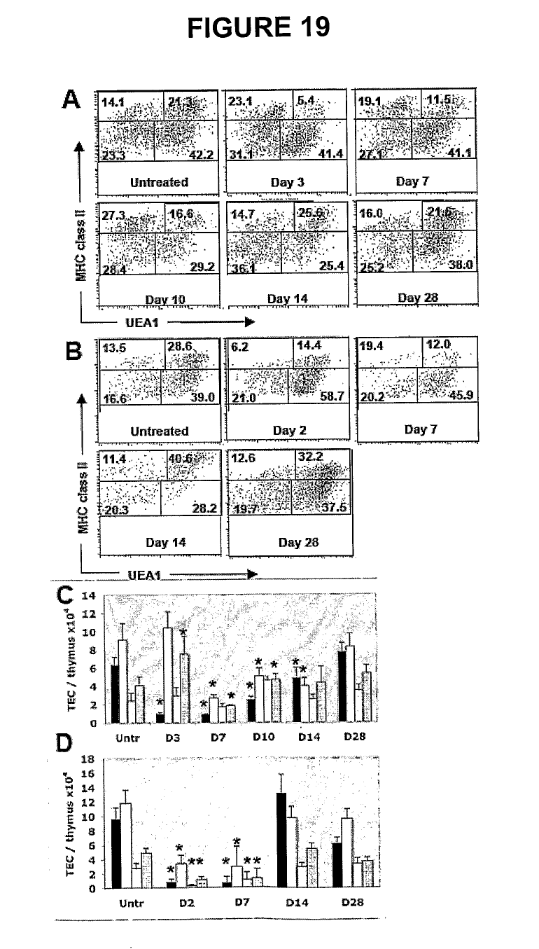

In order to further test the hypothesis that mTEC-lo cells seed mTEC-hi, TEC regeneration was studied in other models of thymic damage. Using the clinically relevant chemotherapeutic agent Cyclophosphamide and corticosteroid Dexamethasone, severe thymic involution was induced (FIG. 19 for thymus cellularity). Each agent ablated all TEC subsets in male and female mice, respectively (FIG. 19C). Again, however, the mTEC-hi subset proved most sensitive to treatment (FIG. 19A) and although Cyclophosphamide and CsA each affected mTEC-hi to a comparable extent, these cells took longer to recover after cyclophosphamide treatment (compare FIG. 19 to FIG. 13). Importantly, mTEC-lo were proportionally reduced concomitant with mTEC-hi recovery at days 10 and 14 (FIG. 19A) indicating a differentiation of mTEC-lo cells to an mTEC-hi phenotype. However, numerically, a different pattern of recovery was noted, where mTEC-lo and mTEC-hi both increased in number between days 7 and 10, with mTEC-hi expanding proportionally faster, as befits a transit-amplifying population (FIG. 19C). The same proportional skewing of mTEC subsets, with the same kinetics of recovery, were noted following Dexamethasone treatment of female mice (FIG. 19B, D).

Phenotypic Correlation with Other Putative TEC Stem Cell (PTSC) Markers

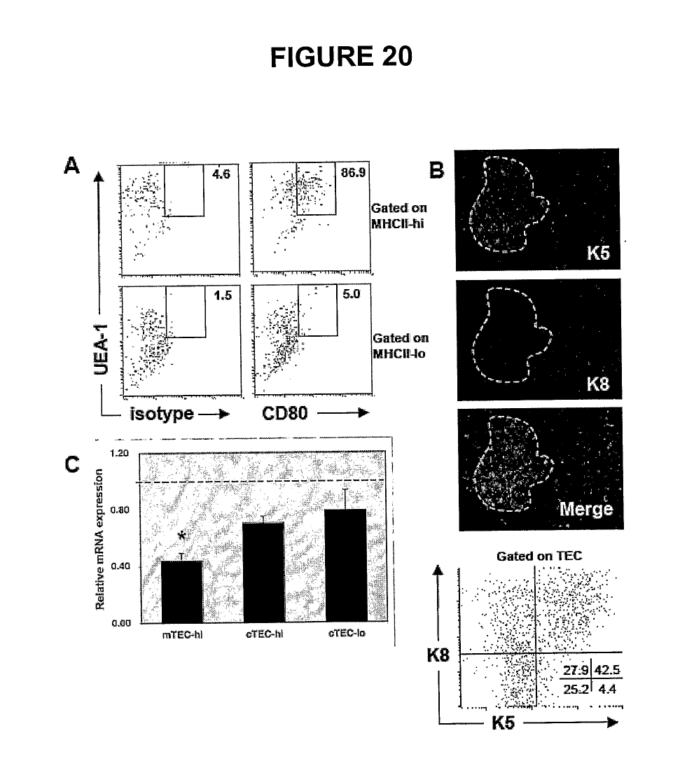

Expression of CD80, MHCII, K5, K8 and transcription factor p63 was assessed in TEC subsets to assess their relevance to the putative Ly51^{lo/-}, MTS24⁺ mTEC-lo progenitor subset previously defined. Flow cytometric analysis revealed that the majority of mTEC-lo cells in the postnatal mouse thymus are CD80⁻, while most mTEC-hi are CD80⁺ (FIG. 20A). K8 was expressed throughout medullary regions and by most mTEC (FIG. 20B) rather than being largely restricted to cTEC, and therefore was of little use as a marker of medullary progenitor cells. Consistent with this, although mTEC-lo expressed the highest p63 transcript levels compared to other TEC subsets, there was not enormous difference between this and cTEC subsets (FIG. 20C). Expression levels of p63 were found to increase in whole TEC following cyclophosphamide treatment but its broad expression suggests that p63 does not specifically denote epithelial stem cell ability in the thymus.

Together, these experiments allowed definition of the mTEC regenerative pathway for the first time in vivo. When mTEC-lo were not directly damaged, they differentiated, showing numerical loss, to restore mTEC-hi by day 10 of recovery, regardless of the prior length of CsA treatment. Regeneration of Aire expression—clinically important to restoration of tolerance—was swift and preferential, and the same pattern of TEC regeneration held true across all models of thymic damage so far assessed. Together, these data demonstrate that the mTEC-lo subset of TEC can rapidly differentiate, without division, into mTEC-hi in vivo following thymic damage, and that normal thymic selection mechanisms are quickly restored in mice following chemotherapy and immunosuppression.

### Example 3

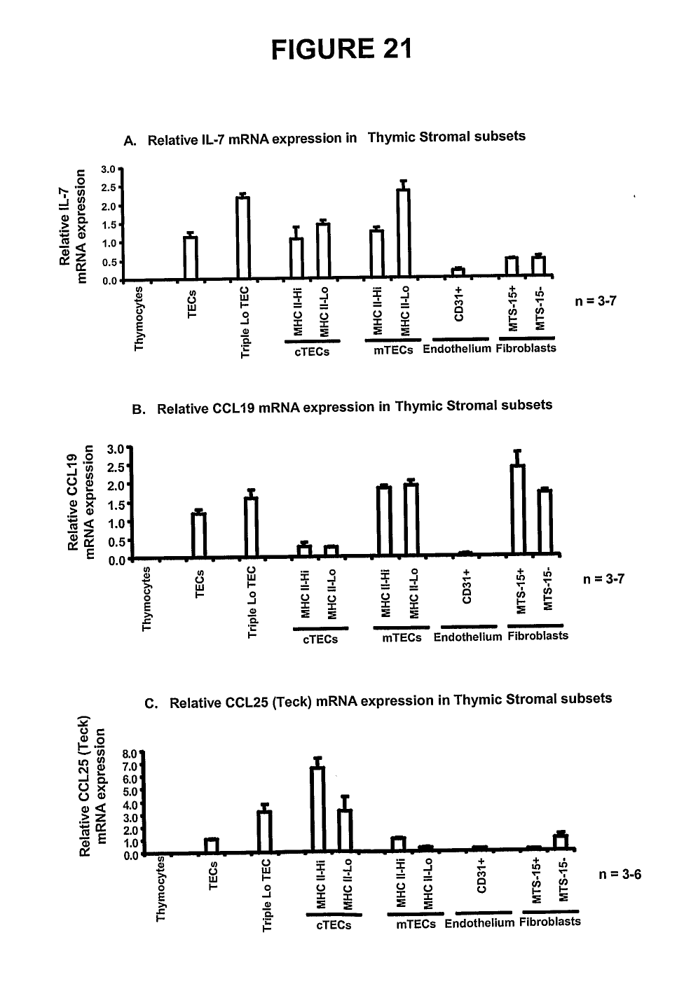

Transcriptional analysis was performed on purified adult (6-7 wks) thymic stromal cell subsets. Transcript levels were normalised to GAPDH. Transcript expression levels are presented as relative to total thymic epithelial cell (TEC) levels which were set to 1 (FIG. 21).
(A) IL7 transcripts were found in all TEC subsets and to a less degree in fibroblasts. Approximately 2 fold more IL7 was found in the Triple Lo TEC subset that expresses low levels of MHCII and both cortical and medullary markers and medullary TECs (mTEC) that expresses low levels of MHCII. Transcript was present to a lesser degree in both MTS15+ and MTS15-fibroblasts. No transcript was present in thymocytes and very little in endothelial cells.
(B) CCL19 transcript expression was expressed mostly by mTEC, Triple Lo and fibroblasts. CCL19 is a chemokine important in attracting cortical thymocytes to the medulla region. The finding that the Triple Lo subset expresses high levels of CCL19, similar to mTEC, indirectly suggests they may reside adjacent to the cortico-medullary junction. Virtually no transcript was present in thymocytes and endothelial cells and very little in cTECs.
(C) CCL25 (TECK) transcript expression is highest in cTEC and Triple Lo cells. CCL25 is thought to be involved in attracting precursors into the thymus. The finding that Triple Lo cells also express CCL25 supports the possibility that they reside around the cortico-medullary junction where precursors enter the thymus.

Graphs represent averages from 3-7 independently prepared templates.

Stromal cell subsets are defined by the following phenotype:

### TEC: CD45-MHCII+

### Triple Lo: CD45⁻MHCII^{lo} Ly51^{lo}UEA1^{lo}

cTEC MHCII-lo: CD45⁻EpCam⁺Ly51⁺MHCII-lo
cTEC MHCII-hi: CD45⁻EpCam⁺Ly51⁺MHCII-hi
mTEC MHCII-lo: CD45⁻EpCam⁺UEA1⁺MHCII-lo
mTEC MHCII-hi: CD45⁻EpCam⁺UEA1⁺MHCIII-hi
CD31+endothelium: CD45⁻MHCII⁻CD31⁺

### MTS15+Fibroblasts: CD45⁻MHCII⁻CD31⁻PDGFRa⁺MTS15⁺

### MTS15−Fibroblasts: CD45⁻MHCII⁻CD31⁻PDGFRa⁺MTS15⁻

### Example 4

### Methods

### LV Construction

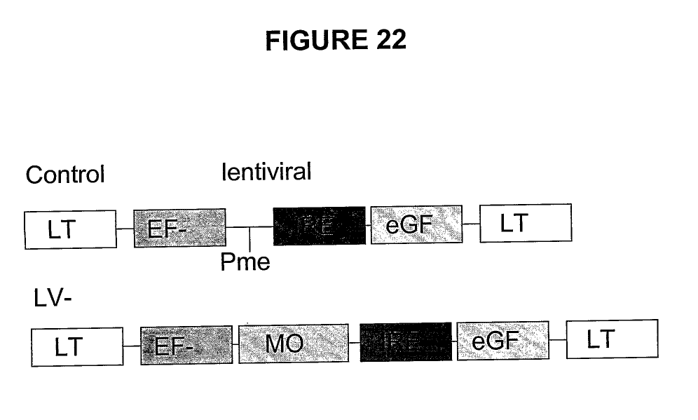

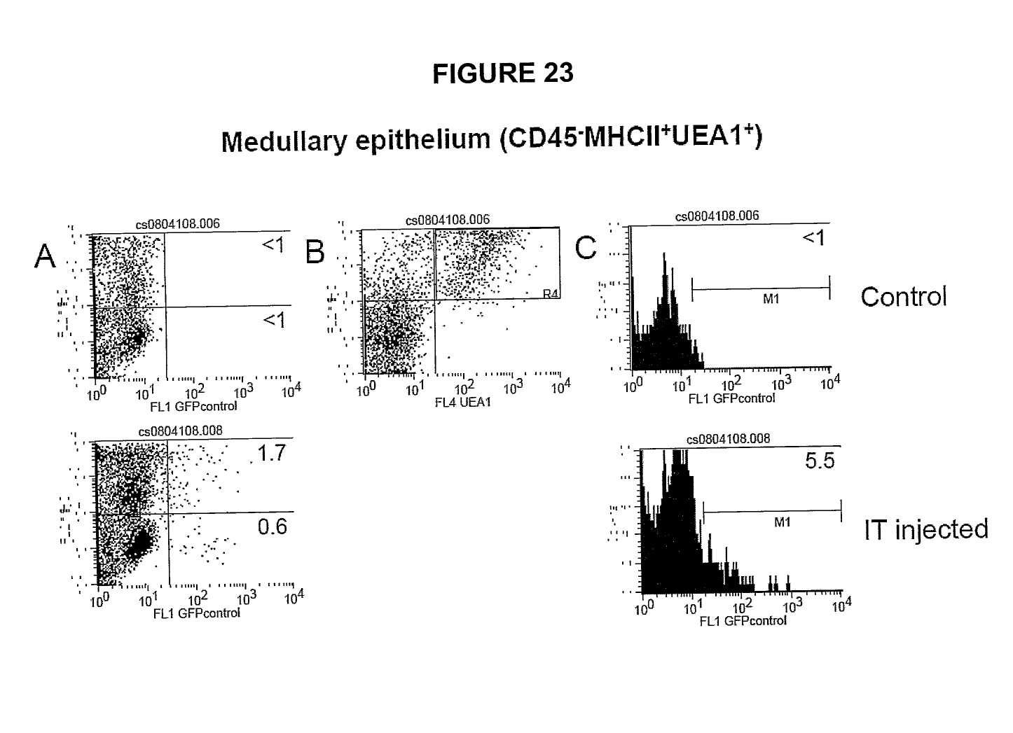

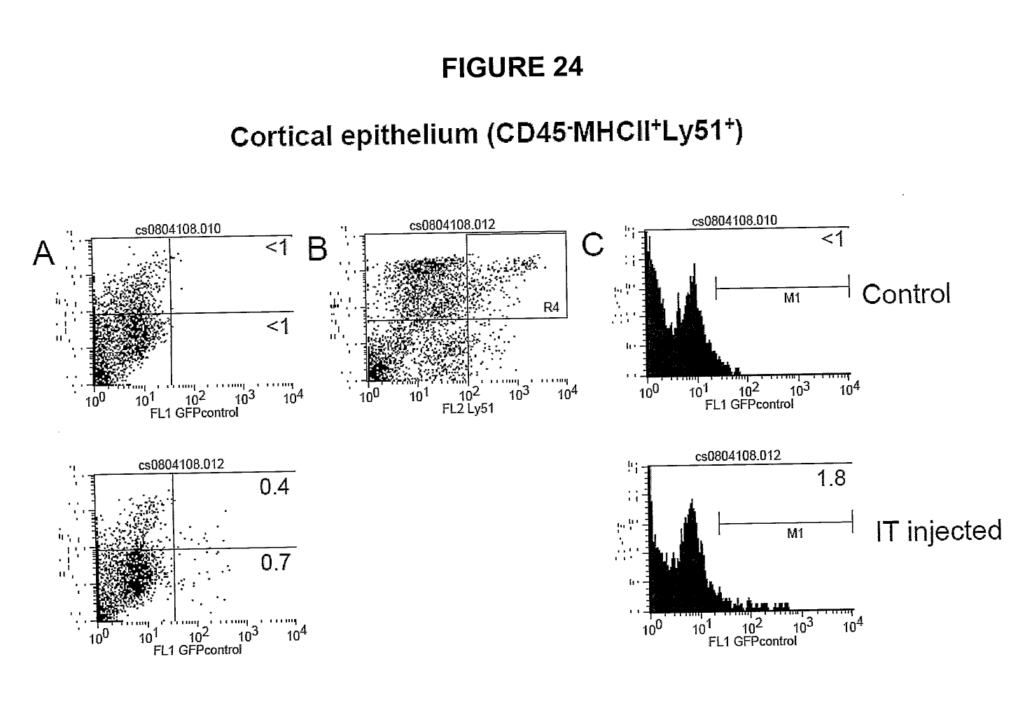

The recombinant lentiviral vector (LV), which is based on pWPI (Bovia et al., 2003) was used in this study. This vector can be used for simultaneously expressing a gene of interest, which is transcribed from an internal EF1a promoter, and enhanced GFP (eGFP), which is translated from an internal ribosomal entry site (IRES) sequence. A full-length mouse myelin oligodendrocyte glycoprotein (MOG) sequence was downstream upstream of the EF-1a promoter sequence into the PmeI as shown in FIG. 22.

### LV Production

Viral stocks were generated by transiently transfecting 293T cells with recombinant lentiviral transfer vectors and the accessory plasmids psPAX2 and pMD.2G using Fugene6 (Roche). Plasmids were purified using a Qiagen maxi plasmid kit (Hilden, Germany). Supernatants were collected after 48 h, concentrated 500-fold by ultracentrifugation (72,000_{gmax}, SW28 rotor, for 140 min at 16° C.), reconstituted in PBS/0.5% BSA and stored at −80° C. Titers were determined by serially diluting viral supernatants and infecting HeLa cells in the presence of 5 mg/mL of protamine sulfate (Sigma-Aldrich) as previously described. Typical titers were in the range of 0.5-1×10⁹ functional infectious particles/mL.

### Intrathymic Injections

Mice were anesthetized using a mixture of ketamine and xylazine. The thymus was made visible after opening the thoracic cage, and 10 μL of lentiviral stock was injected into each lobe using a 30G needle attached to a 50-μL Hamilton syringe (Hamilton, Reno, USA).

### Antibodies

The following pre-titrated antibodies were used in this study: anti-Ly51 (6C3; BD Bioscience, Palo Alto, Calif.), anti-I-A/I-E (M5/114.15.2; BD Bioscience), anti-CD45 (30-F11; BD Bioscience) and biotinylated Ulex Europasus Agglutinin I (Vector laboratories, Burlingame Calif.). Secondary reagents used was streptavidin-conjugated APC (BD Bioscience).

### Thymic Stromal Cell Isolation by Enzymatic Digestion

Thymic stromal cells were isolated as previously described (Gray et al., 2002). In brief, individual thymi were digested using collagenase (Roche, Mannheim, Germany), then collagenase/dispase (Roche) and passed through 100-μm mesh to remove debris.

### Flow Cytometry

Immunofluorescent staining was performed as previously reported. Sample data from ˜1×10⁴ CD45⁻ cells was acquired on a FACScalibur (BD Biosciences, San Jose, Calif.) using up to 4 fluorescent channels and analyzed using CellQuest software (BD Biosciences).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Anderson G, et al. Positive selection of thymocytes: the long and winding road. Immunol Today 1999, 20:464-469
Ardavin C, et al. Thymic dendritic cells and T cells develop simultaneously in the thymus from a common precursor population. Nature 1993, 362:761-763
Bahnson, A. B., et al. Method for Retrovirus-Mediated Gene Transfer to CD34+-Enriched Cells, in GENE THERAPY PROTOCOLS (P. D. Robbins, ed.), Humana Press, pp. 249-263 (1997)
Belmont, J. W. and R. Jurecic. Methods for Efficient Retrovirus-Mediated Gene Transfer to Mouse Hematopoietic Stem Cells, in GENE THERAPY PROTOCOLS (P. D. Robbins, ed.), Humana Press, pp. 223-40 (1997)
Berzins S P, et al. A central role for thymic emigrants in peripheral T cell homeostasis. Proc Natl Acad Sci USA 1999, 96:9787-9791
Beschornere et al. 1991, Cell Immunol 132:505-514
Beschorner et al. 1987, Cell Immunol 110:350-364
Beschorner et al. 1991, Transplantation 52:668-674
Bevan M J. In thymic selection, peptide diversity gives and takes away. Immunity 1997, 7:175-178
Blackburn C C, Manley N R. Developing a new paradigm for thymus organogenesis. Nat Rev Immunol 2004, 4:278-289
Boehm T, Scheu S, Pfeffer K, Bleul CC. Thymic medullary epithelial cell differentiation, thymocyte emigration, and the control of autoimmunity require lympho-epithelial cross talk via LTbetaR. J Exp Med 2003, 198:757-769
Bovia, F., Salmon, P., Matthes, T., Kvell, K., Nguyen, T. H., Werner-Favre, C., Barnet, M., Nagy, M., Leuba, F., Arrighi, J. F., Piguet, V., Trono, D. and Zubler, R. H. (2003). Efficient transduction of primary human B lymphocytes and nondividing myeloma B cells with HIV-1-derived lentiviral vectors. Blood, 101, 1727-33.
Capecchi, M. R. High Efficiency Transformation by Direct Microinjection of DNA Into Cultured Mammalian Cells, Cell 22:479-88 (1980)
Chidgey A P, Boyd R L. Thymic stromal cells and positive selection. APMIS 2001, 109:481-492
Erlich H. A. (1989) Polymerase chain reaction. J Clin Immunol 9(6):437-47
Fehling H J, et al. Crucial role of the pre-T-cell receptor alpha gene in development of alpha beta but not gamma delta T cells. Nature 1995, 375:795-798
Fodor S P, Read J L, Pirrung M C, Stryer L, Lu A T, Solas D. (1991) Light-directed, spatially addressable parallel chemical synthesis. Science 251(4995):767-73
Gabler et al. 2007, Eur J Immunol 37:3363-3372
Gabor M J, et al. Recent thymic emigrants are distinct from most medullary thymocytes. Eur J Immunol 1997, 27:2010-2015
Godfrey D I, et al. The phenotypic heterogeneity of mouse thymic stromal cells. Immunology 1990, 70:66-74
Godfrey D I, Zlotnik A. Control points in early T-cell development. Immunol Today 1993, 14:547-553
Goeddel et al. 1980
Gordon J, Bennett A R, Blackburn C C, Manley N R. Gcm2 and Foxn1 mark early parathyroid- and thymus-specific domains in the developing third pharyngeal pouch. Mech Dev 2001, 103:141-143
Graham, F. L. and Van Der Eb, A. J. A New Technique for the Assay of Infectivity of Human Adenovirus 5 DNA, Virology 52:456-57 (1973)
Gray et al. 2008. Unbiased analysis, enrichment and purification of thymic stromal cells. J Immunol Methods 329:56-66
Gray et al. 2007, J. Exp. Med. 204:2521-2528
Gray, D. H., Chidgey, A. P. and Boyd, R. L. (2002). Analysis of thymic stromal cell populations using flow cytometry. J. Immunol. Methods, 260, 15-28.
Groettrup M, et al. A novel disulfide-linked heterodimer on pre-T cells consists of the T cell receptor beta chain and a 33 kd glycoprotein. Cell 1993, 75:283-294
Groettrup M, et al. A novel disulfide-linked heterodimer on pre-T cells consists of the T cell receptor beta chain and a 33 kd glycoprotein. Cell 1993, 75:283-294
Hugo P, et al. Ontogeny of a novel CD4+ CD8 CD3 thymocyte subpopulation: a comparison with CD4 CD8+ CD3 thymocytes. Int Immunol 1990, 2:209-218
Jameson S C, Bevan M J. T-cell selection. Curr Opin Immunol 1998, 10:214-219
Kaye J, et al. Selective development of CD4+ T cells in transgenic mice expressing a class II MHC-restricted antigen receptor. Nature 1989, 341:746-749
Kazal et al., 1996
Kim S. S, and Vacanti J. P., 1999. Semin Pediatr Surg. 8:119
Klein L, Kyewski B. Self-antigen presentation by thymic stromal cells: a subtle division of labor. Curr Opin Immunol 2000, 12:179-186
Lawn et al. 1981
Manley N R. Thymus organogenesis and molecular mechanisms of thymic epithelial cell differentiation. Semin Immunol 2000, 12:421-428
Matsuzaki Y, et al. Characterization of c-kit positive intrathymic stem cells that are restricted to lymphoid differentiation. J Exp Med 1993, 178:1283-1292
Merkenschlager M, et al. How many thymocytes audition for selection? J Exp Med 1997, 186:1149-1158
Moore T A, Zlotnik A. T-cell lineage commitment and cytokine responses of thymic progenitors. Blood 1995, 86:1850-1860
Moore, A., Basilion, J., Chiocca, E., and Weissleder, R., BBA, 1402:239-249, 1988
Mullis K B, Faloona F A, 1987, Specific synthesis of DNA in vitro via a polymerase-catalyzed chain reaction. Methods Enzymol. 155:335-50
Nehls M, Kyewski B, Messerle M, Waldschutz R, Schuddekopf K, Smith A J, Boehm T. Two genetically separable steps in the differentiation of thymic epithelium. Science 1996, 272:886-889
Nossal G J V. Negative selection of lymphocytes. Cell 1994, 76:229-239
Owen J J, McLoughlin D E, Suniara R K, Jenkinson E J. The role of mesenchyme in thymus development. Curr Top Microbiol Immunol 2000, 251:133-137
Penit C, Vasseur F. Sequential events in thymocyte differentiation and thymus regeneration revealed by a combination of bromodeoxyuridine DNA labeling and antimitotic drug treatment. J Immunol 1988, 140:3315-3323
Pongracz J, Hare K, Harman B, Anderson G, Jenkinson E J. Thymic epithelial cells provide WNT signals to developing thymocytes. Eur J Immunol 2003, 33:1949-1956
Potter, H., Weir, L., and Leder, P. Enhancer-dependent expression of Human Kappa Immunoglobulin Genes Introduced Into Mouse pre-B Lymphocytes by Electroporation, Proc. Natl. Acad Sci. USA 81:7161-65 (1984)
Rodewald H-R, et al. Identification of pro-thymocytes in murine fetal blood: T lineage commitment can precede thymus colonization. EMBO J 1994, 13:4229-4240
Rossi et al. 2007, J Exp Med 204:1267-1272;
Scollay R, Godfrey D I. Thymic emigration: conveyor belts or lucky dips? Immunol Today 1995, 16:268-273; discussion 273-264
Scollay R G, et al. Thymus cell migration. Quantitative aspects of cellular traffic from the thymus to the periphery in mice. Eur J Immunol 1980, 10:210-218
Sebzda E, et al. Selection of the T cell repertoire. Annu Rev Immunol 1999, 17:829-874
Smith H, et al. Neonatal thymectomy results in a repertoire enriched in T cells deleted in adult thymus. Science 1989, 245:749-752
Sprent J, Kishimoto H. The thymus and negative selection. Immunol Rev 2002, 185:126-135
Stepkowski, S. M. 2000. Expert Rev Mol Med 2:1-23
Suniara R K, Jenkinson E J, Owen J J. An essential role for thymic mesenchyme in early T cell development. J Exp Med 2000, 191:1051-1056
Tatsumi Y. et al. Differentiation of thymocytes from CD3 CD4 CD8 through CD3 CD4 CD8+ into more mature stages induced by a thymic stromal cell clone. PNAS1990, 87:2750-2754
Teh H S, et al. Thymic major histocompatibility complex antigens and the alpha beta T-cell receptor determine the CD4/CD8 phenotype of T cells. Nature 1988, 335:229-233
von Boehmer H. Positive selection of lymphocytes. Cell 1994, 76:219-228
Wedemeyer, N., Potter, T., Wetzlich, S, and Gohde, W. Flow Cytometric Quantification of Competitive Reverse Transcriptase-PCR products, Clinical Chemistry 48(9):1398-1405, 2002
Weissleder, R., Moore, A., Ph.D., Mahmood-Bhorade, U., Benveniste, H., Chiocca, E. A., Basilion, J. P. Nature Medicine, 6:351-355, 2000
Wiest D L, et al. Control of early thymocyte development by the pre-T cell receptor complex: a receptor without a ligand? Semin Immunol 1999, 11:251-262
Wu L, et al. CD4 expressed on earliest T-lineage precursor cells in the adult murine thymus. Nature 1991, 349:71-74
Wu M-Y, et al. The suppression of peritoneal cellular immunity in women with endometriosis could be restored after gonadotropin releasing hormone agonist treatment. Am J Reprod Immunol 1996, 35:510-516
Yang, N.-S, and P. Ziegelhoffer. The Particle Bombardment System for Mammalian Gene Transfer, In PARTICLE BOMBARDMENT TECHNOLOGY FOR GENE TRANSFER (Yang, N.-S, and Christou, P., eds.), Oxford University Press, New York, pp. 117-41 (1997)

1. An isolated mammalian thymic epithelial progenitor cell that expresses an MHC II¹⁰ phenotypic profile.

2-66. (canceled)

67. The isolated cell of claim 1, wherein said cell expresses a CD45^{˜} and epcam⁺ phenotypic profile.

68. The isolated cell of claim 1, wherein said cell expresses a UEA1¹⁰ and Ly51^{b} phenotypic profile.

69. The isolated cell of claim 1, wherein said cell expresses a phenotypic profile selected from the group consisting of:
(i) MHC Class II^{b}, CD45″ and epcam⁺;
(ii) MHC Class II¹⁰, CD45^{˜} UEA1′⁰ and Ly51′^{o};
(iii) MHC Class II¹⁰, epcam⁺, UEA1¹⁰ and Ly51¹⁰;
(iv) MHC Class II¹⁰, CD45^{˜} epcam⁺, UEA1¹⁰ and Ly51¹⁰; or
(v) MHC Class II¹⁰, UEA1¹⁰ and Ly51¹⁰.

70. The isolated cell of claim 1, wherein said cell is a thymic cortical progenitor cell expressing a UEA1″^{lo} and Ly51⁺ phenotypic profile.

71. The isolated cell of claim 1, wherein said cell is a thymic medullary progenitor cell expressing a UEA1⁺ and Ly51^{˜} phenotypic profile.

72. The isolated cell of claim 1, wherein said cell is a thymic cortical progenitor cell expressing a phenotypic profile selected from the group consisting of:
(i) MHC Class II¹⁰, CD45^{˜}, UEAr^{/10} and Ly51⁺; (ii) MHC Class II¹⁰, epcam⁺, IJEA1⁷¹⁰, Ly51⁺; or (iii) MHC Class II¹⁰, CD45″ epcam⁺, UEA1″⁷¹⁰ and Ly51⁺.

73. The isolated cell of claim 1, wherein said cell is a thymic medullary progenitor cell expressing a phenotypic profile selected from the group consisting of:
(i) MHC Class II¹⁰, CD45^{˜}, UEA1⁺ and Ly51^{˜}; (ii) MHC Class II¹⁰, epcam⁺, UEA1⁺, Ly51″; or (iii) MHC Class II¹⁰, CD45^{˜} epcam⁺, UEA1⁺ and Ly51^{˜}.

74. An isolated mammalian thymic epithelial progenitor cell that expresses a MHC Class 1i^{˜} phenotypic profile.

75. The isolated cell of claim 74, wherein said cell expresses a CD45^{˜} and epcam⁺ phenotypic profile.

76. A method for inducing proliferation or differentiation of a thymic epithelial progenitor cell comprising contacting the cell of claim 1 with an agent that induces proliferation or differentiation of said cell.

77. The method of claim 76, wherein said thymic epithelial progenitor cell is differentiated to a mature thymic epithelial phenotype, a thymic cortical phenotype, or a medullary phenotype.

78. The method claim 77, wherein said cell is differentiated to a thymic cortical phenotype.

79. The method of claim 76, wherein said agent is IL-7, keratinocyte growth factor, growth hormone, IGF-I, ghrelin, LHRH₅ culture medium conditioned by bone marrow, culture medium conditioned by mesenchymal cells, bone marrow stroma co-culture or a mesenchymal cell co-culture.

80. The method of claim 79, further comprising generating a cell line from said cells.

81. The method of claim 79, further comprising generating a thymic tissue aggregate from said cells.

82. The method of claim 81, wherein said thymic tissue aggregate is an organoid.

83. The method of claim 79, further comprising co-culturing a T cell precursor with said cell under conditions sufficient to induce T cell precursor maturation.
